(19) United States
(12) Patent Application Publication
(10) Pub. No.: US 2009/0222938 A1
(43) Pub. Date: Sep. 3, 2009

(54) Therapeutic Target Molecules For The Development of Novel Medicaments for Degenerative Diseases

(75) Inventors: Thomas Rohrmeier, Regensburg (DE); Rosemarie Daig, Regensburg (DE)

Correspondence Address: FULBRIGHT & JAWORSKI L.L.P., 600 CONGRESS AVE., SUITE 2400, AUSTIN, TX, 78701, US

(73) Assignee: Neuroprofile GMBH, Regensburg (DE)

(21) Appl. No.: 12/296,641

(22) PCT Filed: Apr. 19, 2007
(86) PCT No.: PCT/DE2007/000697

§ 371 Date: May 8, 2009

(30) Foreign Application Priority Data
Apr. 20, 2006 (DE) 10 2006 018 392.4

(51) Int. Cl. C12Q 1/68; C12N 5/10; A01K 67/027

(52) U.S. Cl. 800/13; 435/6; 435/325

(57) ABSTRACT

The present invention generally relates to the field of therapy, prophylaxis and diagnosis of degenerative diseases, in particular neurodegenerative diseases. Specifically, the present invention relates to genes and proteins, which are regulated in connection with chronic oxidative stress in cells and are applied for therapy, prophylaxis, and diagnosis of degenerative diseases, in particular neurodegenerative diseases. Additionally, the present invention relates to the use of genes and proteins, which are regulated in conjunction with chronic oxidative stress in cells, for the screening of candidate substances to identify prophylactic and/or therapeutic agents, which agents modulate the biologic activity of genes and/or proteins, which genes and/or proteins are activated in conjunction with chronic oxidative stress in cells. Further, the present invention relates to methods for diagnosis of degenerative diseases, in particular neurodegenerative diseases, and methods for identifying prophylactic and/or therapeutic agents, which agents modulate the biologic activity of genes and/or proteins, which genes and/or proteins are activated in conjunction with chronic oxidative stress in cells. Further, the present invention relates to kits performing the methods of diagnosis.

The present invention generally relates to the field of therapy, prophylaxis and diagnosis of degenerative diseases, in particular neurodegenerative diseases. Specifically, the present invention relates to genes and proteins, which are regulated in conjunction with chronic oxidative stress in cells and are applied for therapy, prophylaxis, and diagnosis of degenerative diseases, in particular neurodegenerative diseases. Additionally, the present invention relates to the use of genes and proteins, which are regulated in conjunction with chronic oxidative stress in cells, for the screening of candidate substances to identify prophylactic and/or therapeutic agents, which agents modulate the biologic activity of genes and/or proteins, which genes and/or proteins are activated in conjunction with chronic oxidative stress in cells. Further, the present invention relates to methods for diagnosis of degenerative diseases, in particular neurodegenerative diseases, and methods for identifying prophylactic and/or therapeutic agents, which agents modulate the biological activity of genes and/or proteins, which genes and/or proteins are activated in conjunction with chronic oxidative stress in cells. Further, the present invention relates to kits performing the methods of diagnosis.

Aerobic organisms, among others, use oxidation reactions to gain energy from food and provide metabolites for maintaining the entire catabolic and anabolic metabolism. Thereby, reactive oxygen species (ROS) and reactive nitrogen species (RNS) are continuously forming in the cells such as superoxide anion, hydroxyl radicals, hydrogen peroxide, peroxic nitrites, and nitric oxide. The occurrence or rather formation of these reactive molecules has to be regulated precisely, in order to prevent an uncontrolled oxidation and nitration, respectively, of biomolecules such as proteins, DNA, and lipids in the cells. Having a disequilibrium towards ROS and/or RNS, the cells are subject to oxidative stress, which may result in an uncontrolled and undesired manner of modification of biomolecules and with it dying (degeneration) of the cells. Neurons are particularly susceptible to oxidative stress due to their high energy consumption and high metabolic activity.

The dying of neurons may have disastrous and irreversible effects for the affected human. The dying of neurons, for example, may occur as a result of stroke, cardiac infarction, traumatic brain and bone marrow injuries, infections, inflammatory reactions, excitotoxicity, ischemia, hypoxia, or other restriction supplies of the brain and the bone marrow, respectively. Moreover, dying of neurons occurs in neurodegenerative diseases such as Alzheimer's disease, Lewy body diseases such as Parkinson's disease, diffuse Lewy body diseases, Huntington's disease, amyotrophic lateral sclerosis (ALS), prion diseases (PrD), Creutzfeldt-Jakob disease, Down syndrome, frontotemporal dementia (FTD), corticobasal degenerations, multi-infarct dementias, progressive supranuclear palsy, multiple system atrophy and Korsakoff's syndrome. A dying of neurons may also be a result of medicament effects. Accordingly, a more prolonged administration of neuroleptic drugs for treating psychotic conditions in patients may result in a chronic tardive dyskinesia, which is caused by the increase of concentration of free radicals in the affected neurons and subsequently causing degeneration of the neurons.

The cause of selective and progressive dying of neurons in neurodegenerative diseases is not revealed. The diseases occur as autosomal inherited forms each at a level of maximal 10% and then affect the patients before their 65^{th} birthday (“early onset”). Alzheimer's disease is characterized by intracellular, mainly hyperphosphorylated, of tau protein consisting, neurofibrillar tangles (NFT), and extracellular depositions of amyloid β (Aβ) 40-42 in senile plaques (SP). The result is a selective loss of cholinergic neurons. However, plaque-free forms of Alzheimer's disease do also exist. The inherited forms are caused predominantly by mutations in genes coding the Aβ precursor protein (APP) and presenillin (PS1 and PS2, respectively). Based on this result, the amyloid hypothesis considers an altered Aβ homeostasis resulting in progressive Aβ depositions and Aβ aggregations as a cause for the dying of cholinergic neurons.

The Parkinson's symptomatology is caused by dopamine deficit as a result of dying neurons in the Substantia nigra of the brain. On a cellular level, the occurring of Lewy bodies can be observed, predominantly consisting of aggregated α-synuclein, yet as well containing other proteins such as ubiquitin C-terminal hydrolase. A familial cumulation of Parkinson's disease has been associated with more than 10 chromosomal regions, wherein mutations in genes coding α-synuclein (PARK1), Parkin (PARK2), and DJ-1 (PARK7) are clearly disease causing.

ALS is caused by a progressive loss of motoneurons. 20% of the familial forms of the disease are associated with more than 90 mutations in the Cu—Zn superoxide dismutase (SOD).

Huntington's disease is caused by a fatal extension of a CAG trinucleotide sequence repeat in the gene coding the Huntington-protein. This extension leads to the formation of a glutamine-rich abnormal Huntington-protein accumulating intracellularly and forming aggregates, among others, in striatal neurons.

The predominant quantity of neurodegenerative diseases, however, occurs without involvement of the each known mutations as a sporadic form after the age of 65 (“late onset”). They are caused by a multifactorial cooperation of environmental influences and endogenous factors. Age is considered as the main risk.

In recent years, cell damaging oxidative stress by ROS and RNS is increasingly considered as a disease causing or promoting factor. Accordingly, protein nitration, protein carbonylation, glycoxidation, and lipid peroxides have been detected frequently in brain samples of Alzheimer's and Parkinson's disease. Additional evidences are a compensatory upregulation of anti-oxidative enzymes and enhanced oxidation of RNA and DNA, respectively, as well as a decreased ability repairing such damages. In Parkinson's disease furthermore, a reduced level of the mitochondrial electron transport chain complex I and intracellular thiols as well as an increase of iron can be found. Animal models support the oxidative stress hypothesis in the onset of Parkinson's disease. Accordingly, the administration of 6-OH-dopamine or 1-methyl-4-phenyl-1,2,3,6-tetrahydropyridine (MPTP) induces the degeneration of dopaminergic neurons, as does a chronic pesticide exposition. The DJ-1 protein coded by the PARK7-region adopts an anti-oxidative function in the cell. It is also known, that the α-synuclein aggregation is caused by oxidative stress. In ALS, nitrotyrosines are frequently observed in the bone marrow. In Huntington's disease, 8-hydroxydeoxyguanosine and nitrotyrosine are observed frequently in the striatum as oxidative stress markers.

Oxidative stress is therefore in the pathogenesis of neurodegenerative diseases a critical link between exogenic factors, such as environmental toxic compounds, and endogenous factors, such as genetic predisposition. In addition, it is known, that the efficiency of radical detoxification systems, such as the glutathione (GSH) system, is decreasing in seniority. A therapy directly interfering against this stress in the cellular protection mechanisms and thereby acting neuroprotectively, would be desirable and could prospectively prevent disease progression.

No effective medicaments exist currently for neurodegenerative diseases. The applied agents antagonize just the symptoms. They cannot stop the progression of the disease, however. To some extent, substantial side effects are resulting, such as dyskinesias, confusional conditions, etc. The medication is aiming to substitute the missing neurotransmitter and, respectively, maximize the function of the yet surviving neurons, of which quantity is dramatically decreased at the onset of the symptoms, e.g. by more than 50% in Alzheimer's disease.

60-80% of Alzheimer's patients do not respond to the group of acetylcholinesterase inhibitors. The only alternative at hand is the NMDA receptor antagonist memantin. In clinical development are, among others, selective ligands for the nicotinic acetylcholine receptors and NMDA receptors, inhibitors of the P— and γ-secretase, respectively, as well as of tau aggregation, a vaccination against β-amyloid, non-steroidal anti-inflammatory medicaments, cholesterol level decreasing medicaments (statins) as well as unspecific antioxidants (among others, spin trapping agents).

In Parkinson's disease, the combination of Levodopa and decarboxylase inhibitors is effective just for a period of about 5 years. Further, dopamine agonists or inhibitors of the monoamino-oxidases or the catechol-O-methyltransferase are administered. In clinical development are, among others, neuron growth factors, neuroimmunophilins, unspecific antioxidants, kinase inhibitors (CEP 1347) as well as cell replacement therapies (among others, Spheramine®).

In ALS, the NMDA receptor agonist riluzol is applied for symptomatic therapy. In doing so, a very slight extension of the survival life time of 2 months is observed, wherein the disease results in death between 2-5 years after diagnosis.

Hence, there is a huge need of new medicaments for neurodegenerative diseases with higher efficiency, specificity, and acceptability. Overall, a plenty of therapy approaches exists pursuing anti-inflammatory, anti-aggregatory, anti-excitatory, anti-apoptotic, anti-oxidative, neurotrophic-regenerative as well as transcription/translation modifying strategies. A broad-based therapeutic approach, applying at several stages of the degenerative cascade as far as possible, seems to be currently most promising in view of the complexity of the pathophysiological processes.

Previous anti-oxidative therapy strategies for neurodegenerative diseases use only unspecific anti-oxidants (such as vitamin E, idebenon). These, however, did not result in an amelioration of the disease symptomatology. All these approaches are lacking targeted modulation of cell-endogenous genes and proteins exerting a crucial function in accomplishing the causes or consequences of oxidative stress and acting thereby neuroprotectively. The most promising chances identifying these genes and proteins, respectively, are conditions, in which cells are struggling against oxidative stress, however they are still able to survive, i.e. they are exposed to chronic and especially to sublethal oxidative stress conditions.

In view of the prior art, the problem of the present invention is to provide genes and their products, respectively, which are regulated in conjunction with chronic oxidative stress in cells. Further, the problem of the present invention is to identify agents by means of these genes and their products, respectively, for therapy and/or prophylaxis of degenerative, in particular neurodegenerative diseases.

This problem is solved by the subject-matters defined in the patent claims.

It is understood, that “oxidative stress” relates to the formation of reactive oxygen species and, respectively, reactive nitrogen species in cells or tissue. Oxygen species may be superoxide anion, hydroxyl radical, and H₂O₂. Nitrogen species may be peroxinitrite and nitric oxide. These species form, for example, in reactions of the electron transport chain in the mitochondria of each cell. The risk of cell damaging increases, e.g. when a high metabolic activity prevails in the brain. As a rule, the oxygen species are eliminated by anti-oxidative enzymes, such as superoxide dismutase, catalase, or glutathione peroxidase. If this elimination is not fully accomplished, hydroxyl radicals, superoxide anions and hydrogen peroxide are forming, which may cause severe cell damages and cell death, and finally, for example, neurologic degenerative diseases.

The term “chronic oxidative stress” or “chronic sublethal oxidative stress”, as used herein, refers to the increase of at least one reactive oxygen or nitrogen compound in cells caused by an impact of oxidative stressor(s) compared to a control condition, wherein the increase persists at least 4 hours and up to 35 days or longer.

The term “oxidative stressor”, as used herein, refers to a molecule generating oxidative stress in cells.

The terms “derivative” or “variant” of nucleic acids, as used herein, refer to nucleic acid sequences exhibiting one or more deletions, substitutions, additions, insertions, and/or inversions, to a comparing nucleic acid, as known to the person skilled in the art.

The terms “derivative” or “variant” of proteins, as used herein, refer to amino acid sequences exhibiting one or more deletions, substitutions, additions, insertions, and/or natural and unnatural protein modifications, respectively, to a comparing protein, as known to the person skilled in the art (e.g., glycosylation or GPI anchor).

The terms “homologous sequence” or “homology”, as used herein, refer to a nucleic acid or protein sequence having significant similarity to a comparing sequence or fragments thereof, wherein the nucleic acids and proteins, respectively, exhibiting these homologous sequences exhibit an activity or partial activity comparable to the activity of nucleic acids and proteins, respectively, of the comparing sequence. Nucleic acid sequences are accounted as homologous sequences, which hybridize with comparing sequences or fragments of these comparing sequences under stringent or little stringent conditions (for stringent or little stringent conditions see Sambrook et al., Molecular Cloning, Cold Spring Harbour Laboratory (1989), ISBN 0-87969-309-6). An example for stringent hybridization conditions is: hybridizing in 4×SSC at 65° C. (alternatively in 50% formamide and 4×SSC at 42° C.), followed by several washing steps in 0.1×SSC at 65° C. for one hour in total. An example for little stringent hybridization conditions is: hybridizing in 4×SSC at 37° C., followed by several washing steps in 1×SSC at room temperature. Further, nucleic acid or protein sequences of fragments thereof should be accounted as homologous sequences, which exhibit a significant similarity to the nucleic acid and amino acid sequences, which are used as comparing sequences, using the similarity algorithm BLAST (Basic Local Alignment Search Tool, Altschul et al., Journal of Molecular Biology 215, 403-410 (1990). As used herein, sequences are referred to as significantly similar, which, for example, exhibit a probability of P<10⁻⁵ using standard parameters in the Blast service of NCBI, when comparing to the comparing sequences or fragments thereof.

The term “modulator”, as used herein, refers to an agent having the ability to alter, in particular to increase or decrease the expression rate of a gene and/or the biologic activity of a protein. The alteration of expression rate or biological activity can be thereby directly determined by methods, known to the person skilled in the art, at the nucleic acid level (e.g. produced mRNA) and protein level (e.g. Western blot, 2D gel electrophoresis or FRET).

The term “neurodegenerative disease”, as used herein, refers to degenerative and neurologic diseases such as stroke and multiple sclerosis, as well as neurodegenerative diseases featuring dying neurons such as dementia, Alzheimer's disease, Parkinson's disease, ALS or Huntington's disease. The term neurodegenerative disease relates also to those diseases, in which neurodegenerative processes play a role, e.g. cell degeneration caused by effect of medicaments, such as tardive dyskinesia, or psychiatric diseases such as schizophrenia or depression.

The terms “therapeutic target molecule” or “drug target”, as used herein, refer to genes or proteins, which are utilized by directly influencing their expression rate or biological activity by means of a binding molecule or a substance for therapy, diagnosis, healing, delay and/or prophylaxis of diseases.

The present invention relates to genes and proteins, respectively, which are regulated during chronic sublethal oxidative stress conditions and belong to the cytoprotective arsenal of cells. The present invention is aiming to use these genes and proteins, respectively, as well as derived derivatives, variants, homologues, and fragments thereof, as well as antibodies targeted against the latter for methods of therapy, prophylaxis, and diagnosis of degenerative diseases, in particular neurodegenerative diseases, as well as for methods for identifying prophylactic and/or therapeutic agents modulating the biological activity of these genes and/or proteins. Further, the present invention provides diagnostic kits and the genes and proteins, which are regulated in conjunction with chronic oxidative stress in cells. The kits, genes, and proteins are used to prevent, to give therapy, or to diagnose degenerative diseases and in particular neurodegenerative diseases at an early stage by means of appropriate measures, and to decrease the risk of such a disease by means of this diagnosis, respectively.

In a first aspect, the present invention provides nucleic acids representing genes, which are regulated in eukaryotic cells in conjunction with chronic oxidative stress. Next, the present invention provides the proteins coded by these genes. Further, the present invention provides also such proteins, which are regulated in conjunction with chronic oxidative stress, without acting at the regulation on the nucleic acid level. In particular, the present invention relates to homologues, derivatives, variants, and fragments of nucleic acids as well as of proteins coded by the nucleic acids.

Preferably, the genes, which are regulated in eukaryotic, preferably in human cells in conjunction with chronic oxidative stress, code for MAC30 (Meningioma-associated protein), BRI3 (synonym: I3, pRGR2), G1P3, LOC222171, CUE domain containing 1 (CUEDC1), Niemann-Pick disease type C2 (NPC2), stearyl-CoA desaturase (SCD; Delta-9-Desaturase) and isopentenyl-diphosphate delta isomerase 1 protein (IDI1; =isopentenyl diphosphate dimethylallyl diphosphate isomerase 1). Further, the present invention relates to the transcript variants of the corresponding genes.

The nucleic acid sequences of MAC30 preferably relate to the cDNA sequences corresponding to the NCBI gene bank/EMBL accession numbers NM_{—}014573, BC045655, BC091504, CR613993, CR590967, CR612870, L19183, BC017362 (see example 1).

The nucleic acid sequences of BRI3 preferably relate to the cDNA sequences corresponding to the NCBI gene bank/EMBL accession numbers BC018737, BC071992, AF041430, AB055977, NM_{—}015379, BC062370, AF106966 (see example 2).

The nucleic acid sequences of G1P3 preferably relate to the cDNA sequences corresponding to the NCBI gene bank/EMBL accession numbers NM_{—}022872, NM_{—}022873, NM_{—}002038, X02492, AK024814, BN000257, BC011601, BC015603 and BT006850 (see example 3).

The nucleic acid sequences of LOC222171 preferably relate to the cDNA sequences corresponding to the NCBI gene bank/EMBL accession numbers BC029131, NM_{—}175887, CR619478, CR608739, CR610203, BC018144, CR604389 (see example 4).

The nucleic acid sequences of CUEDC1 preferably relate to the cDNA sequences corresponding to the NCBI gene bank/EMBL accession numbers NM_{—}017949, AK000746, BC056882, AK000977 (see example 5).

The nucleic acid sequences of Niemann-Pick disease type C2 (NPC2) preferably relate to the cDNA sequences corresponding to the NCBI gene bank/EMBL accession numbers NM_{—}006432, BC002532, CR609490, CR608935, CR605546, CR622486, AK222474, CR624497, CR601885, X67698, CR595914 (see example 6).

The nucleic acid sequences of Stearoyl-CoA Desaturase (SCD; Delta-9-Desaturase) preferably relate to the cDNA sequences corresponding to the NCBI gene bank/EMBL accession numbers S70284, Y13647, NM_{—}005063, BC005807, AK222862, AB208982, BC062303, AF097514, AB032261 (see example 7).

The nucleic acid sequences of IDI1 (Isopentenyl diphosphate dimethylallyl diphosphate isomerase 1) preferably relate to the cDNA sequences corresponding to the NCBI gene bank/EMBL accession numbers NM_{—}004508, BC057827, BC019227, BC022418, BC025375, BC006999, BC005247, AF271720 (see example 8).

The proteins, which are regulated in eukaryotic preferably in human cells in conjunction with chronic oxidative stress, relate to the expression products of the preceding mentioned genes and their transcription variants.

The amino acid sequences of the protein MAC30 preferably relate to the accession numbers NP_{—}055388, AAH91504, AAH45655, AAA16188.

The amino acid sequences of the protein Brain protein i3 (BRI3) preferably relate to the accession numbers AAH18737, AAH71992, AAD05167, BAB32785, NP_{—}056194, AAH62370, AAF18565, O95415. The protein brain protein i3 shall not be mixed up with the protein of the accession number Q9NQX7, having the same designation BRI3 without any similarity to the protein of this invention.

The amino acid sequences of the protein Interferon induced 6-16 protein preferably relate to the accession numbers NP_{—}075010, NP_{—}075011, NP_{—}002029, AAH15603, CAE12275, AAH11601, AAP35496, CAA26322.

The amino acid sequences of the protein LOC222171 preferably relate to the accession numbers AAH29131, NP_{—}787083, EAL24204.

The amino acid sequences of the CUE domain-containing 1 protein preferably relate to the accession numbers NP-060419, BAA91357, AAH56882, BAA91452, Q9NWM3.

The amino acid sequences of the NPC2 protein preferably relate to the accession numbers NP_{—}006423, AAH02532, BAD96194, CAA47928, P61916.

The amino acid sequences of the human SCD protein preferably relate to the accession numbers BAA93510, BAD92219, AAH62303, AAD29870, NP_{—}005054, AAH05807, O00767, BAD96582, AAB30631, CAA73998.

The amino acid sequences of the human isopentenyl-diphosphate delta isomerase 1 protein (IDI1; =isopentenyl diphosphate dimethylallyl diphosphate isomerase 1) preferably relate to the accession numbers Q13907, NP_{—}004499, AAH19227, AAK49435, AAK49434, AAK29357, AAH06999.

Preferably, the invention relates to nucleic acids, exhibiting sequences according to SEQ ID NO:1 to 63, as well as proteins, exhibiting sequences according to SEQ ID NO:64 to 113 and their homologues, derivatives, variants and fragments. If it is about homologues of the nucleic acids according to the present invention, the homologues exhibit a homology of at least 80%, preferably a homology of about 85%, 90%, 95%, or 99% to nucleic acids having a sequence according to SEQ ID NO:1-63. If it is about homologues of the proteins according to the present invention, the homologues exhibit an identity of at least 70%, preferably an identity of at least 75%, 85%, 90%, 95% or 99% to the proteins having a sequence according to SEQ ID NO:64-113. Next, the proteins according to the present invention may exhibit modifications. Exemplary modifications are chemically modified amino acids such as naturally non occurring (unnatural) amino acids, deletions, mutations and additions in the amino acid sequence, fusions of proteins with heterologous polypeptides (fusion proteins) as well as chemical and biological modifications of amino acids by means of naturally occurring and non occurring structures such as glycosylations, GPI anchor and/or lipidations.

The nucleic acid molecules according to the present invention may be naturally or non naturally occurring genomic DNA, RNA, cDNA, microRNA, siRNA, as well as homologues, derivatives, fragments and variants, in particular alternative splice variants thereof, or modified, in particular transcriptionally or chemically modified nucleic acids or Peptide Nucleic Acids (PNAs) and the like of it.

In a further aspect, the present invention relates to oligonucleotides, which may hybridize selectively as a probe or primer to the nucleic acids according to the present invention and may be used for detecting and/or amplifying these nucleic acid molecules in biological sample material. The oligonucleotides may be DNA, RNA, or PNAs. In the case of DNA or RNA, the oligonucleotides consist of at least 6, preferably 6-50, 10-45, 12-40, 15-35, 15-30, 20-45, 25-40 in succession following nucleotides or may exhibit a complementary antisense nucleotide sequence to nucleic acids according to the present invention. The oligonucleotides may be modified, e.g. coupled to an enzyme, reacting with a chromophoric, fluorescent, or luminescent substrate, or linked to a molecule of the following: dye, fluorescence, luminescence, or radioactive molecule, and/or mass-spectrometrically agents (isotopentags). Next, the modification of the oligonucleotide may be one or more modification(s) of the bond between the single nucleotides, for example phosphorothioate or methylphosphonate. The oligonucleotides according to the present invention may be used in nucleic acid biochips, in particular electronic biochips in the methods according to the present invention.

In a further aspect, the present invention relates to the use of nucleic acids, which are regulated in conjunction with chronic oxidative stress in cells, and, respectively, the use of the proteins coded by these nucleic acids in medical research for example in the research of neurodegenerative diseases. In particular, the present invention relates to the use of nucleic acids, which are regulated in conjunction with chronic oxidative stress in cells, and, respectively, of the proteins coded by these nucleic acids as target molecules (drug targets) for identifying agents, which modulate the biological activity of genes and/or proteins, which genes and/or proteins are regulated in conjunction with chronic oxidative stress in cells. In particular, the present invention thereby relates to the use of nucleic acids, which exhibit a sequence according to SEQ ID NO:1 to 63, as well as homologues, derivatives, variants, and fragments thereof, and proteins, which exhibit a sequence according to SEQ ID NO:64 to 113, as well as homologues, derivatives, variants, and fragments thereof. The nucleic acids and proteins for use may also exhibit modifications, as defined herein.

The use according to the present invention allows the identification of therapeutic and/or prophylactic agents, which are employed against diseases and/or (chronic) disease-related conditions, which are in conjunction with oxidative stress, in particular cancer, cardiovascular diseases, atherosclerosis, diabetes mellitus, inflammatory diseases of the immune system, rheumatoid arthritis, inflammatory intestine diseases, premature aging processes, degenerative and neurologic diseases such as stroke and multiple sclerosis as well as neurodegenerative diseases featuring dying neurons such as dementia, Alzheimer's disease, Parkinson's disease, ALS or Huntington's disease, and, respectively, which prevent these diseases and/or (chronic) disease-related conditions.

In a further aspect, the present invention relates to the use of nucleic acids or proteins, which are regulated in conjunction with chronic oxidative stress in cells, for identifying, monitoring, nosological classification (categorization of disease stages), treatment, diagnosis, and/or prognostic assessment of diseases or disease-related conditions, which are caused by means of oxidative stress of cells. In particular, the present invention relates to the use of nucleic acids, which exhibit a sequence according to SEQ ID NO:1 to 63, as well as homologues, derivatives, variants, and fragments thereof, as well as the use of proteins, which exhibit a sequence according to SEQ ID NO:64 to 113, as well as homologues, derivatives, variants, and fragments thereof.

In one embodiment, the present invention relates to a diagnostic method for detecting and/or analyzing nucleic acids, which are regulated in conjunction with chronic oxidative stress in cells.

The method comprises thereby the following steps:
a) isolating nucleic acids from a sample,
b) producing cDNA or optionally previously synthesizing cRNA for linear amplification,
c) adding oligonucleotides targeted against a gene, which is regulated in conjunction with chronic oxidative stress, and subsequently amplificating the cDNA of step b),
d) analyzing the amplification products of step c).

If a signal enhancing is required for the analysis, the amplified products of step c) may be hybridized to chemically modified oligonucleotides or complementary nucleic acid sequences on a biochip.

In a further embodiment, the present invention relates to a diagnosis method for detecting and/or analyzing nucleic acids, which are regulated in conjunction with chronic oxidative stress in cells, comprising the following steps:
a) isolating nucleic acids from a sample, wherein the RNA is optionally directly labelled,
b) adding oligonucleotides targeted against a gene, which is regulated in conjunction with chronic oxidative stress, and subsequently hybridizing to the isolated nucleic acids of step a),
c) analyzing the hybridization signals.

The isolated sample may be a biological sample, for example, tissue samples such as of brain, or body fluids such as blood, saliva, serum, or cerebrospinal fluid (CSF). The sample may also be DNA or RNA previously obtained from biological material.

The nucleic acids, which are to be detected, may be DNA or RNA, which are regulated in conjunction with chronic oxidative stress in cells, wherein DNA is preferred. Hereby, the nucleic acid, which is to be detected, may be particularly preferred as one or more nucleic acids, which exhibit a sequence according to SEQ ID NO:1 to 63, as well as homologues, derivatives, variants, and fragments thereof. If the nucleic acid is RNA, a reverse transcription of the RNA to cDNA is performed previously to nucleic acid amplification, followed by an additional cDNA synthesis using the produced cRNA. If the nucleic acid is RNA, the reverse transcription and nucleic acid amplification is performed preferably by means of a RT-PCR.

Preferably, the determination of the expression rate level of one or more nucleic acids, which are regulated in conjunction with chronic oxidative stress in cells, is performed by means of the methods according to the present invention. Thereto, total RNA or mRNA is isolated from the sample and the RNA is reversely transcribed into cDNA. After a first cDNA synthesis an in vitro transcription is performed with a DNA dependent RNA polymerase for linear amplification. For direct labelling, alkaline phosphatase, for example, is coupled to the RNA and subsequently the enzymatic activity is detected. Preferably, the cDNA is amplified for this purpose using the methods according to the present invention, wherein a quantitative PCR is performed, as known in the related art. Further, the expression rates of the nucleic acids, which are regulated in conjunction with chronic oxidative stress in cells, may be detected by hybridizing oligonucleotides to the nucleic acids. Preferably, the nucleic acid, of which expression is to be quantified, is thereby one or more nucleic acids exhibiting a sequence according to SEQ ID NO:1 to 63, as well as homologues, derivatives, variants, and fragments thereof.

The determination of the expression rate of the nucleic acids allows, e.g. the diagnosis, whether oxidative stress was occurring in the cells, of which or with which the sample was obtained, consequently allowing a statement about identifying, monitoring, nosological classification, diagnosis and/or prognostic assessment of diseases and disease-related conditions, which are caused by oxidative stress of cells. The methods according to the present invention allow the diagnosis of diseases and/or (chronic) disease-related conditions, which are in conjunction with oxidative stress, in particular cancer, cardiovascular diseases, atherosclerosis, diabetes mellitus, inflammatory diseases of the immune system, rheumatoid arthritis, inflammatory intestine diseases, premature aging processes, degenerative and neurologic diseases such as stroke and multiple sclerosis as well as neurodegenerative diseases featuring dying neurons such as dementia, Alzheimer's disease, Parkinson's disease, ALS, or Huntington's disease. Next, identifying, assessing, and/or monitoring by means of the methods according to the present invention of medicament side effects occurs as a result of oxidative stress caused by the according medicaments.

A further aspect of the present invention is a kit for performing analysis and detection methods according to the present invention, wherein the kit comprises at least one primer pair for performing the nucleic acid amplification according to the method of the present invention. Thereby, the primers of the corresponding primer pairs comprise each DNA sequences hybridizing to nucleic acids, which are regulated in conjunction with chronic oxidative stress in cells. Preferably, the primers comprise each DNA sequences hybridizing to one or more nucleic acid(s) exhibiting a sequence according to SEQ ID NO:1 to 63, as well as homologues, derivatives, variants, and fragments thereof. Further, the kit according to the present invention corresponding to the method for nucleic acid amplification, which is to be performed, may comprise appropriate reagents, such as buffers, nucleotides, and enzymes, e.g. DNA polymerases, as well as at least one appropriate reference nucleic acid(s).

In a further aspect, the present invention relates to the use of proteins, which are regulated in conjunction with chronic oxidative stress in cells, for identifying, monitoring, nosological classification, treatment, diagnosis, and/or prognostic assessment of diseases or disease-related conditions, which are caused by means of oxidative stress of cells. In particular, the present invention thereby relates to the use of proteins exhibiting a sequence according to SEQ ID NO: 64 to 113, as well as homologues, derivatives, variants, and fragments thereof.

In one embodiment the present invention relates to an analysis and/or diagnosis method for quantification of proteins, which are regulated in conjunction with chronic oxidative stress in cells. The method comprises thereby the following steps:
a) performing a quantification of at least one protein, which is regulated in conjunction with chronic oxidative stress in cells, in an isolated sample, preferably of biological material such as brain, CSF, blood, saliva.
b) comparing to the determined quantities of at least one protein of a reference sample, which is regulated in conjunction with chronic oxidative stress in cells, wherein the reference sample is derived from a subject not suffering from a degenerative disease,
   wherein an increased quantity of the at least one protein of the tested sample of step a) in comparison to the quantity of the corresponding protein of the reference sample indicates having a degenerative disease or the risk contracting a degenerative disease.

Preferably, the quantification of at least one protein is performed, which is selected from the group of proteins exhibiting a sequence according to SEQ ID NO:64 to 113, as well as homologues, derivatives, variants, and fragments thereof.

The performance of quantification preferably occurs immunologically, for example by means of an ELISA assay, Western blots, RIA, immunohistochemistry, or immunocytochemistry. Preferably, the protein quantification according to the present invention is performed immunologically using monoclonal or polyclonal antibodies or other molecules, which specifically bind to a protein, which is regulated in conjunction with chronic oxidative stress in cells, and, respectively, bind specifically to a protein, which is selected from the group of proteins exhibiting a sequence according to SEQ ID NO:64 to 113, as well as homologues, derivatives, variants, and fragments thereof. The molecules may be identical to the therapeutic or prophylactic binder, described below, and may be biomolecules or chemicals, in particular small chemical molecules, as described below for modulators of the expression and biological activity, respectively. The quantification preferably may also be performed non immunologically by means of NMR or PET probes. The quantification by means of mass spectrometry is likewise preferred. The proteins, which are to be determined, may exhibit modifications, as defined above.

In one preferred embodiment, the sample is contacted with an antibody or binder, and the quantity of the forming complex of antibodies and proteins, and, respectively, binders and proteins is determined by means of the above mentioned method. The antibodies or binders may be chemically modified. The antibodies or binders may be, for example, covalently coupled to an enzyme reacting with a (chromophoric) substrate, wherein the reaction may be visualized and detected—and therewith the formed complex of antibody and protein, and, respectively, binder and protein—by means of the resulting fluorescence, luminescence, or phosphorescence. Next, the antibodies or binders may be directly linked with dye, fluorescence, luminescence, or radioactive molecules, and, respectively, may contain mass-spectrometrically active isotopes, so that the detection of the formed complex of antibody and protein, and, respectively, binder and protein may be directly visualized without an interposed enzymatic reaction step.

In a further preferred embodiment, protein chips are used for detection of antibodies against proteins, which are regulated in conjunction with chronic oxidative stress in cells, in material of patients, such as blood, serum, CSF, brain, saliva.

In a further preferred embodiment, the protein chips contain antibodies or binders, respectively, for detection of the proteins according to the present invention, which are regulated in conjunction with chronic oxidative stress in cells, in material of patients, such as blood, serum, CSF, brain, saliva.

The quantification of a protein, which is regulated in conjunction with chronic oxidative stress in cells, allows the identification, monitoring, nosological classification, diagnosis and/or prognostic assessment of diseases or disease-related conditions, which are caused by oxidative stress of cells. The method according to the present invention allows the diagnosis of diseases and/or (chronic) disease-related conditions, which are in conjunction with oxidative stress, in particular cancer, cardiovascular diseases, atherosclerosis, diabetes mellitus, inflammatory diseases of the immune system, rheumatoid arthritis, inflammatory intestine diseases, premature aging processes, degenerative and neurologic diseases such as stroke and multiple sclerosis as well as neurodegenerative diseases featuring dying neurons such as dementia, Alzheimer's disease, Parkinson's disease, ALS, or Huntington's disease. Next, identifying, assessing, and/or monitoring of medicament side effects is performed by means of the methods according to the present invention, which occur as a result of oxidative stress caused by the corresponding medicaments.

A further aspect of the present invention is a kit for performing the method according to the present invention for quantification of at least one protein, which is regulated in conjunction with chronic oxidative stress in cells. The kit comprises thereby at least one monoclonal or polyclonal antibody or other binder, which is specific for a protein, which is regulated in conjunction with chronic oxidative stress in cells, and which is preferably specific for a protein, which is selected from the group of proteins exhibiting a sequence according to SEQ ID NO:64 to 113 as well as homologues, derivatives, variants, and fragments thereof. Further, the kit according to the present invention corresponding to the method for protein quantification, which is to be performed, may comprise appropriate reagents such as buffers as well as (an) appropriate reference protein(s). The kit according to the present invention may further contain NMR tags, PET probes, isotopentags for mass spectrometry and/or aptamers.

In a further aspect, the present invention relates to the use of nucleic acids, which are regulated in conjunction with chronic oxidative stress in cells, for identifying therapeutic and/or prophylactic agents, which modulate the expression of nucleic acids, which are regulated in conjunction with chronic oxidative stress in cells, and, respectively, modulate the biologic activity of the proteins coded by these nucleic acids. In particular, the present invention thereby relates to the use of nucleic acids exhibiting a sequence according to SEQ ID NO:1 to 63 and proteins exhibiting a sequence according to SEQ ID NO:64 to 113 as well as their homologues, derivatives, variants, and fragments.

In one embodiment, the present invention relates to a method for identifying therapeutic and/or prophylactic agents, which modulate the expression of nucleic acids, which are regulated in conjunction with chronic oxidative stress in cells, wherein the method comprises the following steps:
a) providing a cell, which expresses a nucleic acid, which is regulated in conjunction with chronic oxidative stress in cells
b) contacting said cell with a candidate substance and
c) comparing the expression of the nucleic acid, which is regulated in conjunction with chronic oxidative stress in cells, to the expression of a nucleic acid, which is regulated in conjunction with chronic oxidative stress in cells, when the candidate substance is not added,
   wherein an alteration of expression of the nucleic acid, which is regulated in conjunction with chronic oxidative stress in cells, indicates, that the candidate substance is a modulator of the nucleic acid, which is regulated in conjunction with chronic oxidative stress in cells.

In a further embodiment, the present invention relates to a method for identifying therapeutic and/or prophylactic agents, which modulate the biologic activity of proteins, which are regulated in conjunction with chronic oxidative stress in cells, wherein the method comprises the following steps:
a) providing a cell containing a protein, which is regulated in conjunction with chronic oxidative stress in cells, and, respectively, immobilizing at least one protein on a carrier material, which is regulated in conjunction with chronic oxidative stress in cells
b) contacting the cell and the protein, respectively, on the carrier material with a candidate substance and
c) detecting the quantity of the candidate substance bound to a protein, which is regulated in conjunction with chronic oxidative stress in cells, by means of biophysical methods such as plasmon surface resonance, FRET, quantitative HPLC, BioAssays, and mass spectrometry,
   wherein binding indicates that it is a potential modulator.

According to the present invention, determination of the expression of the nucleic acid and, respectively, the quantity of the protein expressed by this nucleic acid may be performed by means of an RNA analysis, in particular by means of an Northern blot, an RNA/cDNA hybridization with possible signal enhancing or an RT-PCR, or by means of protein analysis methods, in particular by means of Western blot analysis or ELISA.

Preferably, the method according to the present invention is performed for identifying therapeutic and/or prophylactic agents using gene libraries, expression libraries, natural compound libraries, libraries of small chemical molecules, recombinatorially chemically produced lead structures, and suchlike.

The candidate substances used according to the present invention may be biomolecules or chemicals, in particular small chemical molecules, as defined below.

Examples of biomolecules, which may be active as modulators of expression and biological activity, respectively, are: nucleic acids such as polynucleotides and oligonucleotides, purines, pyrimidines, polypeptides, antibodies, oligosaccharides, polysaccharides, lipids, fatty acids, steroids or structural analogs, fragments or derivatives thereof, and/or combinations thereof.

Examples of modulators of expression are: micro-RNA, siRNA or other molecules of RNA interference (RNAi) known to the person skilled in the art; oligonucleotides, polynucleotides, antisense nucleic acids, PNA, aptamers, or ribozymes, which, e.g., have specific effects on the transcription and/or translation of nucleic acids as transcriptional activators or inhibitors, and, respectively, as translation activators or inhibitors, which nucleic acids are regulated in conjunction with chronic oxidative stress in cells. Modulators of expression may exhibit modifications.

Examples of modulators of biological activity are: polymeric forms of amino acids of any length (e.g. polypeptides, proteins), which, for example, include naturally and non naturally occurring amino acids and occur as a single chain of amino acids or a multimer molecule; polypeptides comprise amino acid analogs, modified or derivatized amino acids; polypeptides with cyclic or bicyclic peptide backbone; fusion proteins, depsipeptides, PNAs, or peptidomimetics. Modulators of biological activity may exhibit modifications.

Modulators of biological activity may have additionally an effect on the binding abilities of said proteins.

Preferred modulators of biological activity are antibodies, for example polyclonal or monoclonal antibodies, which act agonistically, antagonistically, or neutralizingly on the biological activity of proteins, which are regulated in conjunction with oxidative stress in cells and bind to the antibodies. Preferably, the antibodies are of human origin or humanized. Preferably, the antibodies, acting as modulators according to the present invention, comprise at least one of the following domains: variable region of an immunoglobulin, constant region of an immunoglobulin, heavy chain of an immunoglobulin, light chain of an immunoglobulin and antigen binding region of an immunoglobulin.

Next, modulators of biological activity include also active fragments of an antibody binding specifically to an antigen or epitope of a protein, which is regulated in conjunction with oxidative stress in cells. An active fragment may be a fab fragment, an fc fragment, a fragment of the variable domain of the heavy chain or light chain.

Next, modulators of biological activity also include antibodies or active fragments thereof, specifically binding to ligands of a protein, which is regulated in conjunction with oxidative stress in cells, and thereby modulating the activity of the ligand.

Modulators of biological activity of proteins, which are regulated in conjunction with oxidative stress in cells, may be bound to a therapeutic and/or prophylactic agent. This bond may be covalent. Appropriate agents are for example: radioactive isotopes, unspecific antioxidants, neuron growth factors as well as aggregation inhibitors.

Examples of chemicals as modulators of expression of nucleic acids, which are regulated in conjunction with oxidative stress in cells, and, respectively, as modulators of biological activity of proteins coded by these nucleic acids are: chemicals of any chemical class, synthetic, semi-synthetic or naturally occurring, inorganic or organic molecules, small molecules or macromolecular complexes or metallic elements such as lithium, or gases. Examples for the above mentioned small chemical molecules are: small organic compounds with a molecular weight of at least about 30 and less of about 5000 Dalton.

The chemicals and small molecules acting as modulators for the interaction with nucleic acids or proteins, in particular by means of hydrogen bonds may exhibit at least one of the following functional groups: hydroxyl, amino, imino, carboxyl, or carbonyl. Further, the chemicals and small molecules acting as modulators may be or exhibit a monocyclic or polycyclic carbon structure or an heterocyclic structure, and/or an aromatic or polyaromatic structure, which is substituted with at least one of the above mentioned functional groups.

In a preferred embodiment, the agents, modulating the biological activity of proteins, which are regulated in conjunction with chronic oxidative stress in cells, are agents binding to proteins, which are regulated in conjunction with chronic oxidative stress in cells (so called “binder”). In order to identify such binders, protein chips are preferably used comprising proteins, which are regulated in conjunction with chronic oxidative stress in cells, in particular proteins comprising a sequence according to SEQ ID NO:64 to 113 as well as derivatives, variants, homologues, and fragments thereof.

A further aspect of the present invention is a kit for performing the method according to the present invention for identifying therapeutic and/or prophylactic agents, which modulate the expression of nucleic acids, which are regulated in conjunction with chronic oxidative stress in cells, and, respectively, the biological activity of the proteins coded by these nucleic acids, wherein the kit comprises at least one cell expressing a nucleic acid, which is regulated in conjunction with chronic oxidative stress in cells. Preferably, the cell comprises one or more nucleic acid(s) exhibiting a sequence according to SEQ ID NO:1 to 63, as well as homologues, derivatives, variants, and fragments thereof.

The nucleic acids used for the methods according to the present invention, which are regulated in conjunction with chronic oxidative stress in cells, and, respectively, nucleic acids coding for proteins, which are regulated in conjunction with oxidative stress, and preferably exhibiting the sequences according to SEQ ID NO:1 to 63, as well as homologues, derivatives, variants, and fragments thereof, may be functionally linked with regulatory control elements allowing an effective expression, i.e. transcription and/or translation in host cells and/or host organisms. Regulatory control elements are, for example, constitutive, inducible, cell or tissue specific promoters, known to the person skilled in the art. Additional regulatory control elements are terminator sequences and polyadenylation signal sequences. The nucleic acids functionally linked to regulatory control elements may occur in a vector, for example in a plasmid, cosmid, phagemid, viroid, virus, preferably adenovirus and baculovirus. Next, the nucleic acids functionally linked to regulatory control elements may occur together with a marker gene in the host cells and/or host organisms, wherein the marker gene may occur together with the nucleic acids functionally linked to regulatory control elements on a vector or on a second vector differing from the former vector. In a second case, the vector comprising the marker gene together with the vector comprising the nucleic acid functionally linked to the regulatory elements is delivered into the host cell or host organism.

Next, the present invention relates to the use of nucleic acids, which are regulated in conjunction with chronic oxidative stress in cells, and, respectively, nucleic acids coding a protein, which is regulated in conjunction with oxidative stress, and which nucleic acids preferably exhibit sequences according to SEQ ID NO:1 to 63, as well as homologues, derivatives, variants and fragments thereof, for producing transgenic cells and non human organisms, preferably a transgenic non human mammal.

A further aspect of the present invention is a cell comprising at least one recombinant nucleic acid, which is regulated in conjunction with chronic oxidative stress in cells, and, respectively, at least one recombinant nucleic acid coding a protein, which is regulated in conjunction with oxidative stress, and preferably exhibiting the sequences of SEQ ID NO:1 to 63, as well as homologues, derivatives, variants and fragments thereof, wherein the recombinant nucleic acid is functionally linked with regulatory control elements.

A further aspect of the present invention is a non human organism, preferably a mammal comprising at least one recombinant nucleic acid, which is regulated in conjunction with chronic oxidative stress in cells, and, respectively, at least one recombinant nucleic acid, which codes a protein regulated in conjunction with oxidative stress, and which nucleic acid exhibits preferably the sequences of SEQ ID NO:1 to 63, as well as homologues, derivatives, variants, and fragments thereof, wherein the recombinant nucleic acid is functionally linked to regulatory control elements.

A further aspect is the therapeutic use of nucleic acids and proteins in medical science, which are regulated in conjunction with chronic oxidative stress in cells. Preferably thereby, nucleic acids and proteins are used exhibiting the sequences according to SEQ ID NO:1 to 113, as well as their homologues, derivatives, variants, and fragments.

### EXAMPLES

### Example 1

The increase of MAC30-mRNA in NT2-N neuron cells (Andrews P. W., Dev. Biol., 103, pp 285-293, 1984; Pleasure S. J., Page C., Lee V. M.-Y., J. Neurosci., 12, pp 1802-1815, 1992) has been detected in microarray analyses using fluorescence-labelled cDNA after treating the cells under serum-free conditions with chronic sublethal concentrations of the oxidative stressor haloperidol (Sigma; catalogue No. H-1512). Appropriate conditions for an optimal survival rate of the cells were obtained by means of the Live/Dead-Viability/Cytotoxicity Assay (Molecular Probes) according to the manual of the manufacturer. More than 75% of the neurons were alive at a concentration of the oxidative stressor in the range of 1.0-25 μM more than 3 days after the time point of sampling for expression profiling experiments by means of two dimensional gel electrophoresis and DNA microarrays, respectively. The concentration of ROS and RNS as well as the redox status of the cells was obtained for further characterizing the effect on the cells. 2′,7′-dichlorodihydrofluorescein-diacetate (Sigma, catalogue No. D6883) was used for determining total cell quantity of ROS. Cells in a 10 cm plate were scraped off in PBS, an aliquot of 5 μM DCFDA was added and incubated for 1 h at 37° C. Subsequently, the cells were homogenized by sonication, centrifugated (20 minutes, 4° C., 20.000×g), and the fluorescence of the supernatant was measured. The standard was DCF. All data were normalized with the protein concentration. The relative increase of ROS was calculated in relation to the control. Further, ROS was determined by means of dihydroethidium (DHE; Sigma catalogue No. D7008). 5 μM DHE was added into an aliquot of the scraped cells, incubated for 1 hour at 37° C., processed analogous to the DCFDA procedure, and the fluorescence of the supernatant was determined. The protein adjusted alteration of mitochondrial ROS was calculated in relation to the untreated control.

The concentrations of haloperidol used in the various experiments were ranging between 1.0-25 μM. However, the concentrations of the stressor may also be higher or lower than the stated range.

The MAC30-mRNA increase may be quantified by a quantitative real-time RT-PCR analysis (qRTRTPCR) with fluorescence labelling by SYBR Green 1. After an 8-day treatment of the cells, the increase is 50% or more, and may achieve up to 200% or more with longer treatment. The PCR primer pair MAC30-For1 (5′-AAGCCATCTTCCTTAGCCTCCCAAGTA-3′) (SEQ ID NO: 114) and MAC-30-Rev2 (5′-AAAACCCTGTCTCCACACACACAAAAA-3′) (SEQ ID NO: 115) was used for the analysis of the MAC30 mRNA by means of qRTRTPCR. A cDNA fragment was amplified with this primer pair, exhibiting a coding sequence for a polypeptide 69 amino acids in length (accession Nos. NP_{—}055388 und AAH45655). Likewise, a cDNA fragment was amplified with this primer pair, exhibiting a coding sequence for a polypeptide 176 amino acids in length (accession No. AAH91504). Moreover, by means of this primer pair a cDNA fragment was amplified, exhibiting a coding sequence for a polypeptide 189 amino acids in length (accession No. AAA16188). This fragments are parts of the same protein differing in length, which are identical, except for a single amino acid exchange. The differing lengths result from the differing position of the first ATG start codon, which occurs downstream in NP_{—}055388 und AAH45655 due to a sequence variation. In the case of AAA16188, the open reading frame (ORF) begins at the first codon in the sequence, which is not an ATG. In contrast, AAH91504 uses the first ATG of this sequence. Moreover, additional mRNAs may be detected with this primer pair, coding additional MAC30 proteins differing in length and exhibiting a similarity of at least 50%.

### Example 2

The increase of the BRI3-mRNA in NT2-N neurons was detected in microarray analyses using fluorescence labelled cDNA after treatment with Haloperidol (as described in example 1). This increase may be quantified by means of a quantitative real-time RT-PCR analysis (qRTRTPCR) with fluorescence labelling by SYBR Green 1. The increase can be detected 3 days after treatment. After an 8-day treatment of the cells, the increase is already 40% or more and may achieve up to 200% or more with longer treatment. The PCR primer pair BRI3-For1 (5′-CTTTGGGTTCATTTGCTGTTTTG-3′) (SEQ ID NO: 116) and BRI3-Rev2 (5′-CATTAGAAAAAGAGAGCTGGGTGTA-3′) (SEQ ID NO: 117) is suitable for the analysis of the BRI3 mRNA by means of qRTRTPCR. cDNA-BRI3 fragments were amplified with this primer pair, exhibiting coding regions for polypeptides of 125 amino acids in length.

### Example 3

The increase of the G1P3-mRNA in NT2-N neurons may be detected in microarray analyses with fluorescence labeled cDNA after treatment with haloperidol (as described in example 1). The increase was quantified by means of a quantitative real-time RT-PCR analysis (qRTRTPCR) with fluorescence labeling by SYBR Green 1. After 3 days already, a marked increase can be detected, which may be 50% or more after an 8-day treatment, and may achieve up to 400% or more with longer treatment. The PCR primer pair G1P3-For1 (5′-GCTATTCACAGATGCGAACATAGTA-3′) (SEQ ID NO: 118) and GIP3-Rev2 (5′-GGAGAGTGATAGACAAAGTTCTGGA-3′) (SEQ ID NO: 119) is suitable for the analysis of the G1P3 mRNA by means of qRTRTPCR. A cDNA fragment may be amplified with this primer pair exhibiting a coding region for a polypeptide of 134 amino acids in length. Likewise, a cDNA fragment was amplified with this primer pair, exhibiting a coding region for a polypeptide of 138 amino acids in length. Moreover, a cDNA fragment was also amplified with this primer pair, exhibiting a coding region for a polypeptide of 130 amino acids in length. The differences can be explained by minor variations in transcript length in the coding region.

### Example 4

The increase of LOC222171-mRNA in NT2-N neurons may be detected in microarray analyses with fluorescence labelled cDNA after treatment with haloperidol (as described in example 1). This increase was quantified by means of a quantitative real time RT-PCR analysis (qRTRTPCR) featuring fluorescence labelling by SYBR-Green 1. After 15 days of haloperidol treatment, a marked increase can be detected, which is 50% or more and may increase further with longer treatment. The PCR primer pair LOC222171-For1 (5′-TGGCTGTTATTTAGGACTCTGTGGAAA-3′) (SEQ ID NO: 120) and LOC222171-Rev2 (5′-TCCCCCACTCCTTCACTTAAGGTATAA-3′) (SEQ ID NO: 121) is suitable for the analysis of the LOC222171 mRNA by means of qRTRTPCR. A cDNA fragment may be amplified with this primer pair, exhibiting a coding region for a polypeptide of 129 amino acids in length.

### Example 5

The increase of the CUEDC1-mRNA in NT2-N neurons may be detected in microarray analyses with fluorescence labelled cDNA after treatment of the cells with haloperidol (as described in example 1). This increase was further quantified by means of a quantitative real time RT-PCR analysis (qRTRTPCR) with fluorescence labelling by SYBR-Green 1. After 15 days of chronic oxidative stress, an increase of more than 30% was detected, which may increase further with longer treatment. The PCR primer pair CUEDC1-For1 (5′-CTTATTCAGGGACAAGCTGAAACACAT-3′) (SEQ ID NO: 122) and CUEDC1-Rev2 (5′-AGTGTTTCCTCTTTGACTTCCTCATTTT-3′) (SEQ ID NO: 123) is suitable for the analysis of the CUEDC1 mRNA by means of qRTRTPCR. A cDNA fragment may be amplified with this primer pair exhibiting a coding region for a polypeptide of 386 amino acids in length. Likewise, a cDNA fragment may be amplified with this primer pair, exhibiting a coding region for a polypeptide of 358 amino acids in length. The capability detecting two polypeptides results from AK000977 exhibiting a deletion in the coding region, whereby a polypeptide forms being 28 amino acids shorter, which polypeptide differs, apart from that, from other primary structures only by a conservative amino acid exchange at the amino acid 47 of the mature protein.

### Example 6

The increase of the NPC2-mRNA in NT2-N neurons was detected in microarray analyses with fluorescence labelled cDNA after treatment with haloperidol (as described in example 1). This increase was quantified by means of a quantitative real time RT-PCR analysis (qRTRTPCR) with fluorescence labelling by SYBR-Green 1. After 3 days already, a significant increase of the NPC2-mRNA was detected in comparison to untreated neurons. After 15 days of chronic sublethal oxidative stress, the increase is 50% or more and may increase further with longer treatment. The PCR primer pair NPC2-For1 (5′-AATTAACTGCCCTATCCAAAAAGAC-3′) (SEQ ID NO: 124) and NPC2-Rev2 (5′-CAGAAGAGACTTTGGTTTTTGTCAT-3′) (SEQ ID NO: 125) is suitable for the analysis of the NPC2-mRNA by means of qRTRTPCR. A cDNA fragment was amplified with this primer pair, exhibiting a coding region for a polypeptide of 151 amino acids in length. Likewise, a cDNA fragment was amplified with this primer pair, exhibiting a coding region for a polypeptide of 151 amino acids in length, which differs from the former polypeptide by two conservative amino acid exchanges.

### Example 7

The increase of the SCD-mRNA was detected in microarray analyses with fluorescence labelled cDNA after treatment of NT2-N neurons with haloperidol (as described in example 1). This increase was quantified by means of a quantitative real time RT-PCR analysis (qRTRTPCR) with fluorescence labelling by SYBR-Green 1. After a 15 day treatment, an increase of the SCD-mRNA of 50% or more was detected in comparison to untreated neurons. The PCR primer pair SCD-For1 (5′-AAAGATGATATATATGACCCCACCT-3′) (SEQ ID NO: 126) and SCD-Rev2 (5′-CCAAGTGTAGCAGAGACATAAGGAT-3′) (SEQ ID NO: 127) is suitable for the analysis of the SCD-mRNA by means of qRTRTPCR. A cDNA fragment was amplified with this primer pair, exhibiting a coding region for a polypeptide of 359 amino acids in length. Likewise, a cDNA fragment was amplified with this primer pair, exhibiting a coding region for a polypeptide of 366 amino acids in length. Moreover, a cDNA fragment was amplified with this primer pair, exhibiting a coding region for a polypeptide of 355 amino acids in length. Further, a cDNA fragment was amplified with this primer pair, exhibiting a coding region for a polypeptide of 237 amino acids in length.

### Example 8

The increase of the IDI1 protein (IDI1=Isopentenyl-diphosphate delta isomerase 1; =Isopentenyl diphosphate dimethylallyl diphosphate isomerase 1) was detected with a two dimensional polyacrylamid gel electrophoresis after treatment of NT2-N neurons with haloperidol (as described in example 1) and subsequent mass spectrometric identification of the protein spot. The quantity of the IDI1-protein increases by 30% or more during a 15 day treatment with haloperidol.

1-17. (canceled)

18. A method for identifying therapeutic and/or prophylactic agents, which modulate the expression of nucleic acids and/or proteins, which are regulated in conjunction with chronic oxidative stress in cells and wherein the nucleic acids exhibit a sequence according to SEQ ID NO:1 to 63 and the proteins exhibit a sequence according to SEQ ID NO:64 to 113 as well as their homologues, derivatives, variants, and fragments, wherein the method comprises the following steps:
a) providing a cell expressing a nucleic acid having a sequence according to SEQ ID NO:1 to 63 as well as their homologues, derivatives, variants, and fragments,
b) contacting the cell with a candidate substance and
c) comparing subsequently to step b) the expression of the nucleic acid to the expression of a nucleic acid, which exhibits a sequence according to SEQ ID NO:1 to 63 as well as their homologues, derivatives, variants, and fragments, when the candidate substance is not added,
wherein an alteration of expression of the nucleic acid indicates, that the candidate substance is a modulator of the nucleic acid.

19. A method for identifying therapeutic and/or prophylactic agents, which modulate the biological activity of proteins, which exhibit a sequence according to SEQ ID NO:64 to 113 as well as their homologues, derivatives, variants, and fragments, wherein the method comprises the following steps:
a) providing a cell containing a protein, which has a sequence according to SEQ ID NO:64 to 113 as well as their homologues, derivatives, variants, and fragments, and, respectively, immobilizing at least one protein, which has a sequence according to SEQ ID NO:64 to 113 as well as their homologues, derivatives, variants, and fragments, on a carrier material,
b) contacting the cell and the protein, respectively, on the carrier material with a candidate substance, and
c) detecting the quantity of the candidate substance bound to the protein by means of biophysical methods such as plasmon surface resonance, FRET, quantitative HPLC, BioAssays, and mass spectrometry,
wherein binding indicates that it is a potential modulator.

20. A kit for identifying therapeutic and/or prophylactic agents, which modulate the expression of nucleic acids, which are regulated in conjunction with chronic oxidative stress in cells, and, respectively, the biological activity of the proteins coded by these nucleic acids, wherein the nucleic acids exhibit a sequence according to SEQ ID NO:1 to 63 and the proteins exhibit a sequence according to SEQ ID NO:64 to 113 as well as their homologues, derivatives, variants, and fragments, and wherein the kit comprises at least one cell expressing a nucleic acid having a sequence according to SEQ ID NO:1 to 63 as well as their homologues, derivatives, variants, and fragments.

21. A method for detecting and/or analyzing nucleic acids having a sequence according to SEQ ID NO:1 to 63 as well as their homologues, derivatives, variants, and fragments, which method comprises the following steps:
a) isolating nucleic acids from a sample,
b) producing cDNA or optionally previously synthesizing cRNA for linear amplification,
c) adding oligonucleotides targeted against a gene, which is regulated in conjunction with chronic oxidative stress, and subsequently amplificating the cDNA of step b),
d) analysing the amplification products of step c).

22. The method according to claim 21, wherein the analysis is performed by means of hybridizing chemically modified oligonucleotides or complementary nucleotide sequences on a biochip.

23. A method for detecting and/or analyzing nucleic acids having a sequence according to SEQ ID NO:1 to 63 as well as their homologues, derivatives, variants, and fragments, which method comprises the following steps:
a) isolating nucleic acids from a sample, wherein the RNA is optionally directly labelled,
b) adding oligonucleotides targeted against a nucleotide sequence according to SEQ ID NO:1 to 63 as well as their homologues, derivatives, variants, and fragments, and subsequently hybridizing to the isolated nucleic acids of step a),
c) analyzing the hybridization signals.

24. A method for analysis and/or diagnosis methods for the quantification of proteins having a sequence according to SEQ ID NO:64 to 113 as well as their homologues, derivatives, variants, and fragments, which methods comprise the following steps:
a) performing a quantification of at least one protein, which has a sequence according to SEQ ID NO:64 to 113 as well as their homologues, derivatives, variants, and fragments, in an isolated sample,
b) comparing to the determined quantities of at least one protein of a reference sample, which protein has a sequence according to SEQ ID NO:64 to 113 as well as their homologues, derivatives, variants, and fragments, wherein the reference sample is derived from a subject not suffering from a degenerative disease,
wherein an increased quantity of the at least one protein of the tested sample of step a) in comparison to the quantity of the corresponding protein of the reference sample indicates having a degenerative disease or the risk contracting a degenerative disease.

25. The method according to claim 21, wherein the sample is a biological sample.

26. The method according to claim 23, wherein the sample is a biological sample.

27. The method according to claim 24, wherein the sample is a biological sample.

28. The method of claim 25, wherein the biological sample is a tissue sample.

29. The method of claim 28, wherein the tissue sample is from brain

30. The method of claim 25, wherein the biological sample is a body fluid.

31. The method of claim 30, wherein the body fluid is blood, saliva, serum, or cerebrospinal fluid (CSF).

32. (canceled)

33. A kit comprising at least one primer pair, wherein the primers of the at least one primer pair hybridized to nucleic acids, which exhibit a sequence according to SEQ ID NO:1 to 63 as well as their homologues, derivatives, variants, and fragments.

34. A kit comprising at least one monoclonal or polyclonal antibody or other binder, which is specific for a protein, which exhibits a sequence according to SEQ ID NO:64 to 113 as well as their homologues, derivatives, variants, and fragments, and optionally an appropriate reference protein as well as NMR-tags, PET-probes, isotopentags for mass spectrometry, and/or aptamers.

35. A cell comprising at least one recombinant nucleic acid exhibiting one of SEQ ID NO:1 to 63, as well as their homologues, derivatives, variants, and fragments, wherein the recombinant nucleic acid is functionally linked with regulatory control elements.

36. A non-human transgenic organism comprising at least one recombinant nucleic acid exhibiting one of SEQ ID NO:1 to 63, as well as their homologues, derivatives, variants, and fragments, wherein the recombinant nucleic acid is functionally linked with regulatory control elements.


SEQUENCE LISTING (127 sequences)

SEQ ID NO: 1 - DNA, 2133 bp, Homo sapiens
  ref <308>: DATABASE ACCESSION NUMBER: Genbank / NM_014573
  ref <309>: DATABASE ENTRY DATE: 2006-02-16
  ref <313>: RELEVANT RESIDUES IN SEQ ID NO: (1)..(2133)
tggggttccagttgcctctctcgggtcctgcccatgcctcccccgctcctaacccaactttagttcggtgttttcctcgggctttgtctctatcccagccccttctcccaagcggcgggggcgaacgctttgtttcctttaaagtcacttcttccctgggtttaaggtttcccgcgattccacctctggccggttgtcagtgtctgagggaagtagtctggatgaagtggtgtccaaagtcacatgtcagcatggatccgcatcctttttatttgacaagtttagaaacctgctgaagtggtatgctaaggagttcaaagacccactgctacaggagcccccagcctggtttaagtcctttctgttttgcgagcttgtgtttcagctgcctttctttcccattgcaacgtatgccttcctcaaaggaagctgcaagtggattcgaactcctgcaatcatctactctgttcacaccatgacaaccttaattccgatactctccacatttctgtttgaggatttctccaaagccagtggtttcaagggacaaagacctgagactttgcatgaacggttaacccttgtgtctgtctatgccccctacttactcatcccattcatacttttaattttcatgttgcggagcccctactacaagtatgaagagaaaagaaaaaaaaaatgaaggaaacaaccactggcccagggtagagatgcctacagggtggttgcttgttggatacaatacaaggaacactgctcagaacccacgtcttcagcagcatttgaaacactggcagcaatgcacaagagcaagatggtgtcaggaaccatgtcaaaccctcaccttcttccatttttttttttttttttaagacagtctcactctgttgcccaggctggagtaaagggcagtggcatgatctcggctcactgcaacctccgcctcctgggctcaagccatcttccttagcctcccaagtagctagaactacaggtgtgtaccaacacgtatggctaatttgttttgttttttttgtgtgtgtggagacagggttttgccatgttgcccaggttggtctcgaacgcctaggctcaagtgatctgcccacctcagtctccctaagtgctgggattacagacgtgaaccactgggcccagcccaaaccttcaccttctaagggcactgggatgaacggaccgatcggcttgagggtgggcaaaggggtgtgggctaggttataaggaagtggtaccaaataactgtgtgcctgagttccaccgcaagattactaaaagcaggaccagaccagaaactgctaaagaacatggcctgtttgacatgttcatgagtcacctgacccacagcatatatgcttgtgactaaaccctccactcctgattctcaagagtgtatcacctgtcagcaaaatgaatagtgggatattttgggccattttaaatgtgaaattttgcctctttaatgttaattcaaaactatatcaatgttttcttgttcccacctctaacccaaggaaaaaagagaaaacatactatgcaaaggaagtttaaacttaagttttccttaagggtcagcccaacaatgactttcagtcaaatggattaaactggaaaatgtttttgtttctgttgtaaacagatcatcctaggcgaaagttttttttgtttgtttgcttttaaattagtttatttctaaatcttagtcttccacatttctagaggccacctgacacaagtccctgtatctgaagtctagcatctcaaggctgatctggaagtgtgctagtatgctccctagtggataacttaatcttttaatacagttccgtcattcccatcttgttttcagaagagaaggtggctacagccaggcataacatatccactgtgtgcatagagggtctcttcacgttgatgcttggcattccatcagctttctctaagtctttgctcaagttcaaccttaaaatgatgttagacaacaggtcccagtcagttccctctattttcacccattttgctcacaagccatattggcccgattagtggtactgtctgactcacgtgtgtgatccaaataaaggtagctgctgaccaaaaaaaaaaaaaaaa

SEQ ID NO: 2 - DNA, 2133 bp, Homo sapiens; identical to SEQ ID NO: 1
  ref <308>: DATABASE ACCESSION NUMBER: Genbank / BC045655
  ref <309>: DATABASE ENTRY DATE: 2006-04-14
  ref <313>: RELEVANT RESIDUES IN SEQ ID NO: (1)..(2133)

SEQ ID NO: 3 - DNA, 2036 bp, Homo sapiens
  ref <308>: DATABASE ACCESSION NUMBER: Genbank / BC091504
  ref <309>: DATABASE ENTRY DATE: 2005-03-30
  ref <313>: RELEVANT RESIDUES IN SEQ ID NO: (1)..(2036)
gcccaaccgacagactatgggggctccggcaaccaggcgctgcgtggagtggctgctgggcctctacttcctcagccacatccccatcaccctgttcatggacctgcaggcggtgctgccgcgcgagctctacccagtcgagtttagaaacctgctgaagtggtatgctaaggagttcaaagacccactgctacaggagcccccagcctggtttaagtcctttctgttttgcgagcttgtgtttcagctgcctttctttcccattgcaacgtatgccttcctcaaaggaagctgcaagtggattcgaactcctgcaatcatctactctgttcacaccatgacaaccttaattccgatactctccacatttctgtttgaggatttctccaaagccagtggtttcaagggacaaagacctgagactttgcatgaacggttaacccttgtgtctgtctatgccccctacttactcatcccattcatacttttaattttcatgttgcggagcccctactacaagtatgaagagaaaagaaaaaaaaaatgaaggaaacaaccactggcccagggtagagatgcctacagggtggttgcttgttggatacaatacaaggaacactgctcagaacccacgtcttcagcagcatttgaaacactggcagcaatgcacaagagcaagatggtgtcaggaaccatgtcaaaccctcaccttcttccattttttttttttttaagacagtctcactctgttgcccaggctggagtaaagggcagtggcatgatctcggctcactgcaacctccgcctcctgggctcaagccatcttccttagcctcccaagtagctagaactacaggtgtgtaccaacacgtatggctaatttgttttgttttttttgtgtgtgtggagacagggttttgccatgttgcccaggttggtctcgaacgcctaggctcaagtgatctgcccacctcagtctccctaagtgctgggattacagacgtgaaccactgggcccagcccaaaccttcaccttctaagggcactgggatgaacagaccgatcggcttgagggtgggcaaaggggtgtgggctaggttataaggaagtggtaccaaataactgtgtgcctgagttccaccgcaagattactaaaagcaggaccagaccagaaactgctaaagaacatggcctgtttgacatgttcatgagtcacctgacccacagcatatatgcttatgactaaaccctccactcctgattctcaagagtgtatcacctgtcagcaaaatgaatagtgggatattttgggccattttaaatgtgaaattttgcctctttaatgttaattcaaaactatatcaatgttttcttgttcccacctctaacccaaggaaaaaagagaaaacatactatgcaaaggaagtttaaacttaagttttccttaagggtcagcccaacaatgactttcagtcaaatggattaaactggaaaatgtttttgtttctgttgtaaacagatcatcctaggcgaaagttttttttgtttgtttgcttttaaattagtttatttctaaatcttagtcttccacatttctagaggccacctgacacaagtccctgtatctgaagtctagcatctcaaggctgatctggaagtgtgctagtatgctccctagtggataacttaatcgtttaatacagttccgtcattcccatcttgttttcagaagagaaggtggctacagccaggcataacatatccactgtgtgcatagagggtctcttcacgttgatgcttggcattccatcagctttctctaagtctttgctcaagttcaaccttaaaatgatgttagacaacaggtcccagtcagttccctctattttcacccattttgctcacaagccatattggcccgattagtggtactgtctgactcacgtgtgtgatccaaataaaggtagctgctgaaaaaaaaaaaaaaaaaaaaaaaaaaaaaaaaaaaaaaaaaaaaaaaaaaaaaaaaaaaaaaa

SEQ ID NO: 4 - DNA, 1970 bp, Homo sapiens
  ref <308>: DATABASE ACCESSION NUMBER: Genbank / CR613993
  ref <309>: DATABASE ENTRY DATE: 2004-07-22
  ref <313>: RELEVANT RESIDUES IN SEQ ID NO: (1)..(1970)
cgggtccaggcccaaccgacagactatgggggctccggcaaccaggcgctgcgtggagtggctgctgggcctctacttcctcagccacatccccatcaccctgttcatggacctgcaggcggtgctgccgcgcgagctctacccagtcgagtttagaaacctgctgaagtggtatgctaaggagttcaaagacccactgctacaggagcccccagcctggtttaagtcctttctgttttgcgagcttgtgtttcagctgcctttctttcccattgcaacgtatgccttcctcaaaggaagctgcaagtggattcgaactcctgcaatcatctactctgttcacaccatgacaaccttaattccgatactctccacatttctgtttgaggatttctccaaagccagtggtttcaagggacaaagacctgagactttgcatgaacggttaacccttgtgtctgtctatgccccctacttactcatcccattcatacttttaattttcatgttgcggagcccctactacaagtatgaagagaaaagaaaaaaaaaatgaaggaaacaaccactggcccagggtagagatgcctacagggtggttgcttgttggatacaatacaaggaacactgctcagaacccacgtcttcagcagcatttgaaacactggcagcaatgcacaagagcaagatggtgtcaggaaccatgtcaaaccctcaccttcttccattttttttttttttttaagacagtctcactctgttgcccaggctggagtaaagggcagtggcatgatctcggctcactgcaacctccgcctcctgggctcaagccatcttccttagcctcccaagtagctagaactacaggtgtgtaccaacacgtatggctaatttgttttgttttttttgtgtgtgtggagacagggttttgccatgttgcccaggttggtctcgaacgcctaggctcaagtgatctgcccacctcagtctccctaagtgctgggattacagacgtgaaccactgggcccagcccaaaccttcaccttctaagggcactgggatgaacagaccgatcggcttgagggtgggcaaaggggtgtgggctaggttataaggaagtggtaccaaataactgtgtgcctgagttccaccgcaagattactaaaagcaggaccagaccagaaactgctaaagaacatggcctgtttgacatgttcatgagtcacctgacccacagcatatatgcttatgactaaaccctccactcctgattctcaagagtgtatcacctgtcagcaaaatgaatagtgggatattttgggccattttaaatgtgaaattttgcctctttaatgttaattcaaaactatatcaatgttttcttgttcccacctctaacccaaggaaaaaagagaaaacatactatgcaaaggaagtttaaacttaagttttccttaagggtcagcccaacaatgactttcagtcaaatggattaaactggaaaatgtttttgtttctgttgtaaacagatcatcctaggcgaaagttttttttgtttgtttgcttttaaattagtttatttctaaatcttagtcttccacatttctagaggccacctgacacaagtccctgtatctgaagtctagcatctcaaggctgatctggaagtgtgctagtatgctccctagtggataacttaatcttttaatacagttccgtcattcccatcttgttttcagaagagaaggtggctacagccaggcataacatatccactgtgtgcatagagggtctcttcacgttgatgcttggcattccatcagctttctctaagtctttgctcaagttcaaccttaaaatgatgttagacaacaggtcccagtcagttccctctattttcacccattttgctcacaagccatattggcccgattagtggtactgtctgactcacgtgtgtgatccaaat

SEQ ID NO: 5 - DNA, 1960 bp, Homo sapiens
  ref <308>: DATABASE ACCESSION NUMBER: Genbank / CR590967
  ref <309>: DATABASE ENTRY DATE: 2004-07-21
  ref <313>: RELEVANT RESIDUES IN SEQ ID NO: (1)..(1960)
gcccaaccgacagactatgggggctccggcaaccaggcgctgcgtggagtggctgctgggcctctacttcctcagccacatccccatcaccctgttcatggacctgcaggcggtgctgccgcgcgagctctacccagtcgagtttagaaacctgctgaagtggtatgctaaggagttcaaagacccactgctacaggagcccccagcctggtttaagtcctttctgttttgcgagcttgtgtttcagctgcctttctttcccattgcaacgtatgccttcctcaaaggaagctgcaagtggattcgaactcctgcaatcatctactctgttcacaccatgacaaccttaattccgatactctccacatttctgtttgaggatttctccaaagccagtggtttcaagggacaaagacctgagactttgcatgaacggttaacccttgtgtctgtctatgccccctacttactcatcccattcatacttttaattttcatgttgcggagcccctactacaagtatgaagagaaaagaaaaaaaaaatgaaggaaacaaccactggcccagggtagagatgcctacagggtggttgcttgttggatacaatacaaggaacactgctcagaacccacgtcttcagcagcatttgaaacactggcagcaatgcacaagagcaagatggtgtcaggaaccatgtcaaaccctcaccttcttccattttttttttttttttaagacagtctcactctgttgcccaggctggagtaaagggcagtggcatgatctcggctcactgcaacctccgcctcctgggctcaagccatcttccttagcctcccaagtagctagaactacaggtgtgtaccaacacgtatggctaatttgttttgttttttttgtgtgtgtggagacagggttttgccatgttgcccaggttggtctcgaacgcctaggctcaagtgatctgcccacctcagtctccctaagtgctgggattacagacgtgaaccactgggcccagcccaaaccttcaccttctaagggcactgggatgaacagaccgatcggcttgagggtgggcaaaggggtgtgggctaggttataaggaagtggtaccaaataactgtgtgcctgagttccaccgcaagattactaaaagcaggaccagaccagaaactgctaaagaacatggcctgtttgacatgttcatgagtcacctgacccacagcatatatgcttatgactaaaccctccactcctgattctcaagagtgtatcacctgtcagcaaaatgaatagtgggatattttgggccattttaaatgtgaaattttgcctctttaatgttaattcaaaactatatcaatgttttcttgttcccacctctaacccaaggaaaaaagagaaaacatactatgcaaaggaagtttaaacttaagttttccttaagggtcagcccaacaatgactttcagtcaaatggattaaactggaaaatgtttttgtttctgttgtaaacagatcatcctaggcgaaagttttttttgtttgtttgcttttaaattagtttatttctaaatcttagtcttccacatttctagaggccacctgacacaagtccctgtatctgaagtctagcatctcaaggctgatctggaagtgtgctagtatgctccctagtggataacttaatcttttaatacagttccgtcattcccatcttgttttcagaagagaaggtggctacagccaggcataacatatccactgtgtgcatagagggtctcttcacgttgatgcttggcattccatcagctttctctaagtctttgctcaagttcaaccttaaaatgatgttagacaacaggtcccagtcagttccctctattttcacccattttgctcacaagccatattggcccgattagtggtactgtctgactcacgtgtgtgatccaaa

SEQ ID NO: 6 - DNA, 1966 bp, Homo sapiens
  ref <308>: DATABASE ACCESSION NUMBER: Genbank / CR612870
  ref <309>: DATABASE ENTRY DATE: 2004-07-22
  ref <313>: RELEVANT RESIDUES IN SEQ ID NO: (1)..(1966)
cgggtccaggcccaaccgacagactatgggggctccggcaaccaggcgctgcgtggagtggctgctgggcctctacttcctcagccacatccccatcaccctgttcatggacctgcaggcggtgctgccgcgcgagctctacccagtcgagtttagaaacctgctgaagtggtatgctaaggagttcaaagacccactgctacaggagcccccagcctggtttaagtcctttctgttttgcgagcttgtgtttcagctgcctttctttcccattgcaacgtatgccttcctcaaaggaagctgcaagtggattcgaactcctgcaatcatctactctgttcacaccatgacaaccttaattccgatactctccacatttctgtttgaggatttctccaaagccagtggtttcaagggacaaagacctgagactttgcatgaacggttaacccttgtgtctgtctatgccccctacttactcatcccattcatacttttaattttcatgttgcggagcccctactacaagtatgaagagaaaagaaaaaaaaaatgaaggaaacaaccactggcccagggtagagatgcctacagggtggttgcttgttggatacaatacaaggaacactgctcagaacccacgtcttcagcagcatttgaaacactggcagcaatgcacaagagcaagatggtgtcaggaaccatgtcaaaccctcaccttcttccattttttttttttttttaagacagtctcactctgttgcccaggctggagtaaagggcagtggcatgatctcggctcactgcaacctccgcctcctgggctcaagccatcttccttagcctcccaagtagctagaactacaggtgtgtaccaacacgtatggctaatttgttttgttttttttgtgtgtgtggagacagggttttgccatgttgcccaggttggtctcgaacgcctaggctcaagtgatctgcccacctcagtctccctaagtgctgggattacagacgtgaaccactgggcccagcccaaaccttcaccttctaagggcactgggatgaacagaccgatcggcttgagggtgggcaaaggggtgtgggctaggttataaggaagtggtaccaaataactgtgtgcctgagttccaccgcaagattactaaaagcaggaccagaccagaaactgctaaagaacatggcctgtttgacatgttcatgagtcacctgacccacagcatatatgcttatgactaaaccctccactcctgattctcaagagtgtatcacctgtcagcaaaatgaatagtgggatattttgggccattttaaatgtgaaattttgcctctttaatgttaattcaaaactatatcaatgttttcttgttcccacctctaacccaaggaaaaaagagaaaacatactatgcaaaggaagtttaaacttaagttttccttaagggtcagcccaacaatgactttcagtcaaatggattaaactggaaaatgtttttgtttctgttgtaaacagatcatcctaggcgaaagttttttttgtttgtttgcttttaaattagtttatttctaaatcttagtcttccacatttctagaggccacctgacacaagtccctgtatctgaagtctagcatctcaaggctgatctggaagtgtgctagtatgctccctagtggataacttaatcttttaatacagttccgtcattcccatcttgttttcagaagagaaggtggctacagccaggcataacatatccactgtgtgcatagagggtctcttcacgttgatgcttggcattccatcagctttctctaagtctttgctcaagttcaaccttaaaatgatgttagacaacaggtcccagtcagttccctctattttcacccattttgctcacaagccatattggcccgattagtggtactgtctgactcacgtgtgtgatcc

SEQ ID NO: 7 - DNA, 2002 bp, Homo sapiens
  ref <308>: DATABASE ACCESSION NUMBER: Genbank / L19183
  ref <309>: DATABASE ENTRY DATE: 1994-02-02
  ref <313>: RELEVANT RESIDUES IN SEQ ID NO: (1)..(2002)
ctcttctcacatcagcgggtccaggcccaaccgacagactatgggggctccttcaccaggcgctgcgtggagtggctgctgggcctctacttcctcagccacatccccatcaccctgttcatggacctgcaggcggtcgtgccgcgcgagctctacccagtcgagtttagaaacctgctgaagtggtatgctaaggagttcaaagacccactgctacaggagcccccagcctggtttaagtcctttctgttttgcgagcttgtgtttcagctgcctttctttcccattgcaacgtatgccttcctcaaaggaagctgcaagtggattcgaactcctgcaatcatctactctgttcacaccatgacaaccttaattctgatactctccacatttctgtttgaggatttctccaaagccagtggtttcaagggacaaagacctgagactttgcatgaacggttaacccttgtgtctgtctatgccccctacttactcatcccattcatacttttaattttcatgttgcggagcccctactacaagtatgaagagaaaagaaaaaaaaaatgaaggaaacaaccactggcccagggtagagatgcctacagggtggttgcttgttggatacatacaggaacactgctcagaacccacgtcttcagcagcatttgaaacactggcagcaatgcacaagagcaagatggtgtcaggaaccatgtcaaaccctcaccttcttccattttttttttttttttgagacagtctcactctgttgccaggctggagtaaagggcagtggcatgatctcggctcactgcaacctccgcctcctgggctcaagccatcttccttagcctcccaagtagctagaactacaggtgtgtaccaacacgtatggctaatttgttttgttttttttgtgtgtgtggagacagggttttgccatgttgcccaggttggtctcgaacgcctaggctcaagtgatctgcccacctcagtctccctaagtgctgggattacagacgtgaaccactgggcccagcccaaaccttcaccttctaagggcactgggatgaacagaccgatcggcttgagggtgggcaaaggggtgtgggctaggttataaggaagtggtaccaaataactgtgttgcctgagttccaccgcaagattactaaaagcaggaccagaccagaaactgctaaagaacatggcctgtttgacatgttcatgagtcacctgacccacagcatatatgcttatgactaaaccctccactcctgattctcaagagtgtatcacctgtcagcaaaatgaatagtgggatattttgggccattttaaatgtgaaattttgcctctttaatgttaattcaaaactatatcaatgttttcttgttcccacctctaacccaaggaaaaaagagaaaacatactatgcaaaggaagtttaaacttaagttttccttaagggtcagcccaacaatgactttcagtcaaatggattaaactggaaaatgtttttgtttctgttgtaaacagatcatcctaggcgaaagttttttttgtttgtttgcttttaaattagtttatttctaaatcttagtcttccacatttctagaggccacctgacacaagtccctgtatctgaagtctagcatctcaaggctgatctggaagtgtgctagtatgctccctagtggataacttaatcttttaatacagttccgtcattcccatcttgttttcagaagagaaggtggctacagccaggcataacttatccactgtgtgcatagagggtctcttcacgttgatgcttggcattccatcagctttctctaagtctttgctcaagttcaaggttaaaatgatgttagacaacaggtcccagtcagtcccctctattttcacccattttgctcacaagccatattggcccgattagtggtactgtctgactcacgtgtgtgatccaaataaaggtagctgccgggaatt

SEQ ID NO: 8 - DNA, 553 bp, Homo sapiens
  ref <308>: DATABASE ACCESSION NUMBER: Genbank / BC017362
  ref <309>: DATABASE ENTRY DATE: 2006-02-17
  ref <313>: RELEVANT RESIDUES IN SEQ ID NO: (1)..(553)
gcccaaccgacagactatgggggctccggcaaccaggcgctgcgtggagtggctgctgggcctctacttcctcagccacatccccatcaccctgttcatggacctgcaggcggtgctgccgcgcgagctctacccagtcgagtttagaaacctgctgaagtggtatgctaaggagttcaaagacccactgctacaggagcccccagcctggtttaagtcctttctgttttgcgagcttgtgtttcagctgcctttctttcccattgcaacgtatgccttcctcaaaggaagctgcaagtggattcgaactcctgcaatcatctactctgttcacaccatgacaaccttaattccgatactctccacatttctgtttgaggatttctccaaagccagtggtttcaagggacaaagacctgagactttgcatgaacggttaacccttgtgtctgtctatgccccctacttactcatcccattcatacttttaattttcatgttgcggagcccctactacaagtatgaagagaaaagaaaaaaaaaaaaaaaaaaa

SEQ ID NO: 9 - DNA, 731 bp, Homo sapiens
  ref <308>: DATABASE ACCESSION NUMBER: Genbank / BC018737
  ref <309>: DATABASE ENTRY DATE: 2005-03-08
  ref <313>: RELEVANT RESIDUES IN SEQ ID NO: (1)..(731)
gcgtctgcctcagaggggcccgagccacccggtccgccgcgtccccgccgccgccgccgcgtcccccgccggggccgaccgagccgagccgggccggagcggcgggcgcggccgggccgccatggaccacaagccgctgctgcaggagcggccgcccgcctacaacctggaggccggccagggcgactacgcgtgcggcccgcacggctacggcgccatccccgccgcgcccccgccgccgccctacccctacctcgtcacagggatacccacccactatcccagggtctacaacatccacagccggaccgtcacccgctatcctgccaactctatcgtggtcgtaggaggctgtcctgtctgcagggttggggtgctggaggactgcttcaccttcctgggcatcttcctggccatcatcctcttcccctttgggttcatttgctgttttgccttgaggaagcgacgatgccccaactgtggagccaccttcgcttaaagggaacaccaggcccggctttcctacacccagctctctttttctaatgtaaatgttgtgtacaatagttttatttgattaagcttcaggactgttttgtaaagcgaggtgggaccgatgtggcacacgccagctgcggtttcccggagcgtggagaggcagtgctgctgctcccgcccgaggctcatgacaactcaataaagcactgcttttaaaaaaaaaaaaaaaaaaa

SEQ ID NO: 10 - DNA, 1335 bp, Homo sapiens
  ref <308>: DATABASE ACCESSION NUMBER: Genbank / BC071992
  ref <309>: DATABASE ENTRY DATE: 2006-04-18
  ref <313>: RELEVANT RESIDUES IN SEQ ID NO: (1)..(1335)
ccggtccgccgcgtccccgccgccgccgccgcgtcccccgccggggccgaccgagccgagccgggccggagcggcgggcgcggccgggccgccatggaccacaagccgctgctgcaggagcggccgcccgcctacaacctggaggccggccagggcgactacgcgtgcggcccgcacggctacggcgccatccccgccgcgcccccgccgccgccctacccctacctcgtcacagggatacccacccaccatcccagggtctacaacatccacagccggaccgtcacccgctatcctgccaactctatcgtggtcgtaggaggctgtcctgtctgcagggttggggtgctggaggactgcttcaccttcctgggcatcttcctggccatcatcctcttcccctttgggttcatttgctgttttgccttgaggaagcgacgatgccccaactgtggagccaccttcgcttaaagggaacaccaggcccggctttcctacacccagctctctttttctaatgtaaatgttgtgtacaatagttttatttgattaagcttcaggactgttttgtaaagcgaggtgggaccgatgtggcacacgccagctgcggtttcccggagcgtggagaggcagtgctgctgctcccgcccgaggctcatgacaactcaataaagcactgcttttattttttgcagtcttcaatttgaaaaaaaaaaaaaaaaaaactcagtgattggtgattttaggcagcaagaattggagaactcacactgcgaaaagaaaaccttgggtggcagtgatttgaacaccggcagtgctggggcaggacccgagccagcggtggggagagatatagtcagagaacccagcaatacagatccgtccttgggcaaggcgcggtgctggatgatgggtgcggaggaatttgggtaaaggcagagggaaggggtggaggagggccagctcagttgccgaaactctggagtggcggctggctagaaattggtctgtagaaatgaccttgaaaatggagttctggccaggtgcggtggctcacgcctgtaatcccagcactttgggaggccgaggcaggcagatcacgaggtcaggagttcgagaccagcctggccaacatggcaagaccctgtctctactaaaaatacaaaaattagctgggcgtggtggcgcatgcctataatcccagctacttgggaggctgaggtaggagaattgcttgaacctgggaggtggaggttgcagtgaacctagatcacgccactgcattccagcctgggcaacagagcgagactccatcttaaaaaaaaaaaaaaaaaa

SEQ ID NO: 11 - DNA, 758 bp, Homo sapiens
  ref <308>: DATABASE ACCESSION NUMBER: Genbank / AF041430
  ref <309>: DATABASE ENTRY DATE: 2002-04-17
  ref <313>: RELEVANT RESIDUES IN SEQ ID NO: (1)..(758)
gccacccggtccgccgcgtccccgccgccgccgccgcgtcccccgccggggccgaccgagccgagccgggcccgagcggcgggcgcggccgggccgccatggaccacaagccgctgctgcaggagcggccgcccgcctacaacctggaggccggccagggcgactacgcgtgcggcccgcacggctacggcgccatccccgccgcgcccccgccgccgccctacccctacctcgtcacagggatacccacccaccatcccagggtctacaacatccacagccggaccgtcacccgctatcctgccaactctatcgtggtcgtaggaggctgtcctgtctgcagggttggggtgctggaggactgcttcaccttcctgggcatcttcctggccatcatcctcttcccctttgggttcatttgctgttttgccttgaggaagcgacgatgccccaactgtggagccaccttcgcttaaagggaacaccaggcccggctttcctacacccaagctctctttttctaatgtaaatgttgtgtacaatagttttatttgattaagctttaggactgttttgtaaagcgaggtgggaccgatgtggcacacgccagctgcggtttcccggagcgtggagaggcagtgctgctgctcccgcccgaggctcatgacaactcaataaagcactgcttttattttttgcagtcttcaatttgagaaaggtgagaaataatgttttcaataaatgagattcataccatt

SEQ ID NO: 12 - DNA, 709 bp, Homo sapiens
  ref <308>: DATABASE ACCESSION NUMBER: Genbank / AB055977
  ref <309>: DATABASE ENTRY DATE: 2002-07-23
  ref <313>: RELEVANT RESIDUES IN SEQ ID NO: (1)..(709)
gcgtctgcctcagaggggcccgagccacccggtccgccgcgtccccgccgccgccgccgcgtcccccgccggggccgaccgagccgagccgggccggagcggcgggcgcggccgggccgccatggaccacaagccgctgctgcaggagcggccgcccgcctacaacctggaggccggccagggcgactacgcgtgcggcccgcacggctacggcgccatccccgccgcgcccccgccgccgccctacccctacctcgtcacagggatacccacccaccatcccagggtctacaacatccacagccggaccgtcacccgctatcctgccaactctatcgtggtcgtaggaggctgtcctgtctgcagggttggggtgctggaggactgcttcaccttcctgggcatcttcctggccatcatcctcttcccctttgggttcatttgctgttttgccttgaggaagcgacgatgccccaactgtggagccaccttcgcttaaagggaacaccaggcccggctttcctacacccagctctctttttctaatgtaaatgttgtgtacaatagttttatttgattaagcttcaggactgttttgtaaagcgaggtgggaccgatgtggcacacgccagctgcggtttcccggagcgtggagaggcagtgctgctgctcccgcccgaggctcatgacaactcaataaagcactgct

SEQ ID NO: 13 - DNA, 798 bp, Homo sapiens
  ref <308>: DATABASE ACCESSION NUMBER: Genbank / NM_015379
  ref <309>: DATABASE ENTRY DATE: 2005-09-24
  ref <313>: RELEVANT RESIDUES IN SEQ ID NO: (1)..(798)
gcgtctgcctcagaggggcccgagccacccggtccgccgcgtccccgccgccgccgccgcgtcccccgccggggccgaccgagccgagccgggccggagcggcgggcgcggccgggccgccatggaccacaagccgctgctgcaggagcggccgcccgcctacaacctggaggccggccagggcgactacgcgtgcggcccgcacggctacggcgccatccccgccgcgcccccgccgccgccctacccctacctcgtcacagggatacccacccaccatcccagggtctacaacatccacagccggaccgtcacccgctatcctgccaactctatcgtggtcgtaggaggctgtcctgtctgcagggttggggtgctggaggactgcttcaccttcctgggcatcttcctggccatcatcctcttcccctttgggttcatttgctgttttgccttgaggaagcgacgatgccccaactgtggagccaccttcgcttaaagggaacaccaggcccggctttcctacacccagctctctttttctaatgtaaatgttgtgtacaataattttatttgattaagcttcaggactgttttgtaaagcgaggtgggaccgatgtggcacacgccagctgcggtttcccggagcgtggagaggcagtgctgctgctcccgcccgaggctcatgacaactcaataaagcactgcttttattttttgcagtcttcaatttgagaaaggtgagaaataatgttttcaataaatgagattcataccattgttaaaaaaaaaaaaaaa

SEQ ID NO: 14 - DNA, 766 bp, Homo sapiens
  ref <308>: DATABASE ACCESSION NUMBER: Genbank / BC062370
  ref <309>: DATABASE ENTRY DATE: 2005-03-08
  ref <313>: RELEVANT RESIDUES IN SEQ ID NO: (1)..(766)
ccggtccgccgcgtccccgccgccgccgccgcgtcccccgccggggccgaccgagccgagccgggccggagcggcgggcgcggccgggccgccatggaccacaagccgctgctgcaggagcggccgcccgcctacaacctggaggccggccagggcgactacgcgtgcggcccgcacggctacggcgccatccccgccgcgcccccgccgccgccctacccctacctcgtcacagggatacccacccaccatcccagggtctacaacatccacagccggaccgtcacccgctatcctgccaactctatcgtggtcgtaggaggctgtcctgtctgcagggttggggtgctggaggactgcttcaccttcctgggcatcttcctggccatcatcctcttcccctttgggttcatttgctgttttgccttgaggaagcgacgatgccccaactgtggagccaccttcgcttaaagggaacaccaggcccggctttcctacacccagctctctttttctaatgtaaatgttgtgtacaataattttatttgattaagcttcaggactgttttgtaaagcgaggtgggaccgatgtggcacacgccagctgcggtttcccggagcgtggagaggcagtgctgctgctcccgcccgaggctcatgacaactcaataaagcactgcttttattttttgcagtcttcaatttgagaaaggtgagaaataatgttttcaataaatgagattcataccaaaaaaaaaaaaaaaaa

SEQ ID NO: 15 - DNA, 758 bp, Homo sapiens
  ref <308>: DATABASE ACCESSION NUMBER: Genbank / AF106966
  ref <309>: DATABASE ENTRY DATE: 1999-01-15
  ref <313>: RELEVANT RESIDUES IN SEQ ID NO: (1)..(758)
ccccgccgccggccgccgtgtcccccgccggggccgaccgagccgagccgggccggagcggcgggcgcggccgggccgccatggaccacaagccgctgctgcaggagcggccgcccgcctacaacctggaggccggccagggcgactacgcgtgcggcccgcacggctacggcgccatccccgccgcgcccccgccgccgccctacccctacctcgtcacagggatacccacccaccatcccagggtctacaacatccacagccggaccgtcacccgctatcctgccaactctatcgtggtcgtaggaggctgtcctgtctgcagggttggggtgctggaggactgcttcaccttcctgggcatcttcctggccatcatcctcttcccctttgggttcatttgctgttttgccttgaggaagcgacgatgccccaactgtggagccaccttcgcttaaagggaacaccaggcccggctttcctacacccagctctctttttctaatgtaaatgttgtgtacaatagttttatttgattaagcttcaggactgttttgtaaagcgaggtgggaccgatgtggcacacgccagctgcggtttcccggagcgtggagaggcagtgctgctgctcccgcccgaggctcatgacaactcaataaagcactgcttttattttttgcagtcttcaatttgagaaaggtgagaaataatgttttccaataaatgagattcataccattaaaaaaaaaaaaaaaaaa

SEQ ID NO: 16 - DNA, 829 bp, Homo sapiens
  ref <308>: DATABASE ACCESSION NUMBER: Genbank / NM_022872
  ref <309>: DATABASE ENTRY DATE: 2005-10-17
  ref <313>: RELEVANT RESIDUES IN SEQ ID NO: (1)..(829)
gaaccgtttactcgctgctgtgcccatctatcagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtgagaatgcgggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgcctttttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaa

SEQ ID NO: 17 - DNA, 841 bp, Homo sapiens
  ref <308>: DATABASE ACCESSION NUMBER: Genbank / NM_022873
  ref <309>: DATABASE ENTRY DATE: 2005-10-17
  ref <313>: RELEVANT RESIDUES IN SEQ ID NO: (1)..(841)
gaaccgtttactcgctgctgtgcccatctatcagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtgagaatgcgggtaaggatgcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgcctttttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaa

SEQ ID NO: 18 - DNA, 817 bp, Homo sapiens
  ref <308>: DATABASE ACCESSION NUMBER: Genbank / NM_002038
  ref <309>: DATABASE ENTRY DATE: 2005-10-17
  ref <313>: RELEVANT RESIDUES IN SEQ ID NO: (1)..(817)
gaaccgtttactcgctgctgtgcccatctatcagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgcctttttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaa

SEQ ID NO: 19 - DNA, 777 bp, Homo sapiens
  ref <308>: DATABASE ACCESSION NUMBER: Genbank / X02492
  ref <309>: DATABASE ENTRY DATE: 2004-09-09
  ref <313>: RELEVANT RESIDUES IN SEQ ID NO: (1)..(777)
gctccgggctgaagattgcttctcttctctcctccaaggtctagtgacggagcccgcgcgcgcgccaccatgcggcagaaggcggtatccgttttcttgtgctacctgctgctcttcacttgcagtggggtggaggcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgcggtacgccacccacaagtatctcgatagtgaggaggatgaggagtagccagcagctcccagaacctcttcttccttcttggcctaactcttccagttaggatctagaactttgcctttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaa

SEQ ID NO: 20 - DNA, 833 bp, Homo sapiens
  ref <308>: DATABASE ACCESSION NUMBER: Genbank / AK024814
  ref <309>: DATABASE ENTRY DATE: 2003-09-15
  ref <313>: RELEVANT RESIDUES IN SEQ ID NO: (1)..(833)
gttttactcgctgctgtgcccatctatcagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgccttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaacacaaaaaaaaaaaaaaaaaaaa

SEQ ID NO: 21 - DNA, 793 bp, Homo sapiens
  ref <308>: DATABASE ACCESSION NUMBER: Genbank / BN000257
  ref <309>: DATABASE ENTRY DATE: 2006-04-04
  ref <313>: RELEVANT RESIDUES IN SEQ ID NO: (1)..(793)
tatcagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgccttttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaaaaaaa

SEQ ID NO: 22 - DNA, 820 bp, Homo sapiens
  ref <308>: DATABASE ACCESSION NUMBER: Genbank / BC011601
  ref <309>: DATABASE ENTRY DATE: 2005-03-08
  ref <313>: RELEVANT RESIDUES IN SEQ ID NO: (1)..(820)
gcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgccttttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccaaaaaaaaaaaaaaaaaaaaaaaaaaaaaaaaaaaaaaaaaaa

SEQ ID NO: 23 - DNA, 821 bp, Homo sapiens
  ref <308>: DATABASE ACCESSION NUMBER: Genbank / BC015603
  ref <309>: DATABASE ENTRY DATE: 2004-06-29
  ref <313>: RELEVANT RESIDUES IN SEQ ID NO: (1)..(821)
cagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgcctttttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaaaaaaaaaaaaaaaaaaaaaaaaaaaaaaaaaaaaa

SEQ ID NO: 24 - DNA, 393 bp, Homo sapiens
  ref <308>: DATABASE ACCESSION NUMBER: Genbank / BT006850
  ref <309>: DATABASE ENTRY DATE: 2003-03-13
  ref <313>: RELEVANT RESIDUES IN SEQ ID NO: (1)..(393)
atgcggcagaaggcggtatcgcttttcttgtgctacctgctgctcttcacttgcagtggggtggaggcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtag

SEQ ID NO: 25 - DNA, 1717 bp, Homo sapiens
  ref <308>: DATABASE ACCESSION NUMBER: Genbank / BC029131
  ref <309>: DATABASE ENTRY DATE: 2004-06-30
  ref <313>: RELEVANT RESIDUES IN SEQ ID NO: (1)..(1717)
gtggcagcggtgaccgcctcctgggttattttcttttctcccccagctgccccagggttctggtttcccgaatcacttggcaaataaccagcaggtgggcagatgtcttttttgcgttcctacggacgcgtaggcaagttcgatgcgccggagtgaacagccagggtcccatctctcagccggctcccgggcggtgctagcagggaagagggacgcgccggcgaatgaaacactactagcccggaaagccgccctggaccttccacccgggcagcgagtctggcggcccctcgccgccgccccctccgaccagaacagactgcgctgcgagccccgggcttccgccgcagcgcgcagcaattggaggtgccagaggagaccagcagcagcacttgatttagctgggctgcgggaattccgacctgtacactttccagaagcctgatctctccaagtccgcgcccagggactcgagccccggcttccaggttgacccgagcgcgcccccttcgtgctctcggccgcgttcaccgcgcgaggcctccgggtcctgttaagcccgagcagcacaggccgctgcctcagcatccaccccgcagcccacgtgtggcaagccggggaaggggtggagtgaacggccggagaccacgtggagaaaggggccgctttggcccttccatctgggtgccgggagcccctaggccctccggccatggccgacagcggcgatgctggcagctccggcccctggtggaaatcgctcaccaacagcagaaagaaaagcaaggaagccgcagtgggggtgccgcctcccgcccagcccgctcccggggagcccacgccacctgcgccgcccagcccggactggaccagcagctcccgggagaaccagcaccccaatctcctcgggggcgccggcgagccccccaaaccagacaagttatacggggacaaatccggcagcagccgccgcaatttgaagatctcgcgctccggccgctttaaggagaagaggaaagtgcgcgccacgctgctcccggaggcgggcaggtccccggaggaggcaggctttcctggtgacccccacgaggacaagcagtagccccaatagcctgcgcgctccaggactgcctacccagcactaccccaaacccccagttccaaacccgagacttcaggcccgcccccttacgcgttgtctcattccaccaaattcagaatatttacacaatgccttcatgattttatttttctggaaattgaagtgtcaattgggttctcaatatttcatgactccaaggatgcattaaatatttatttgtggtaagagaagatacctgccgcggaggagggtggcataattatttttttttcaaaaagctattctggggccctggccccctggctcctgtcacagctcagcttgaactctgtagcctctcaaatgaagaaggtggctgttatttaggactctgtggaaagcaaatcacagtgctgtttttaatgcctgagaaatgcactttaggggtagacttataccttaagtgaaggagtgggggaggaagaagcattcttaagtagaagcattttaaaataaggcaggattgcttttaatgttaaactacaaaactgtacagcctattttagtgtggactattaaaacccttgcactgtaaggaaaaaaaaaaaaaaaaaaaaaaaaaaaaaaaaaaaaaaa

SEQ ID NO: 26 - DNA, 1717 bp, Homo sapiens; identical to SEQ ID NO: 25
  ref <308>: DATABASE ACCESSION NUMBER: Genbank / NM_175887
  ref <309>: DATABASE ENTRY DATE: 2005-03-02
  ref <313>: RELEVANT RESIDUES IN SEQ ID NO: (1)..(1717)

SEQ ID NO: 27 - DNA, 1599 bp, Homo sapiens
  ref <308>: DATABASE ACCESSION NUMBER: Genbank / CR619478
  ref <309>: DATABASE ENTRY DATE: 2004-07-22
  ref <313>: RELEVANT RESIDUES IN SEQ ID NO: (1)..(1599)
gtttcccgaatcacttggcaaataaccagcaggtgggcagatgtcttttttgcgttcctacggacgcgtaggcaagttcgatgcgccggagtgaacagccagggtcccatctctcagccggctcccgggcggtgctagcagggaagagggacgcgccggcgaatgaaacactactagcccggaaagccgccctggaccttccacccgggcagcgagtctggcggcccctcgccgccgccccctccgaccagaacagactgcgctgcgagccccgggcttccgccgcagcgcgcagcaattggaggtgccagaggagaccagcagcagcacttgatttagctgggctgcgggaattccgacctgtacactttccagaagcctgatctctccaagtccgcgcccagggactcgagccccggcttccaggttgacccgagcgcgcccccttcgtgctctcggccgcgttcaccgcgcgaggcctccgggtcctgttaagcccgagcagcacaggccgctgcctcagcatccaccccgcagcccacgtgtggcaagccggggaaggggtggagtgaacggccggagaccacgtggagaaaggggccgctttggcccttccatctgggtgccgggagcccctaggccctccggccatggccgacagcggcgatgctggcagctccggcccctggtggaaatcgctcaccaacagcagaaagaaaagcaaggaagccgcagtgggggtgccgcctcccgcccagcccgctcccggggagcccacgccacctgcgccgcccagcccggactggaccagcagctcccgggagaaccagcaccccaatctcctcgggggcgccggcgagccccccaaaccagacaagttatacggggacaaatccggcagcagccgccgcaatttgaagatctcgcgctccggccgctttaaggagaagaggaaagtgcgcgccacgctgctcccggaggcgggcaggtccccggaggaggcaggctttcctggtgacccccacgaggacaagcagtagccccaatagcctgcgcgctccaggactgcctacccagcactaccccaaacccccagttccaaacccgagacttcaggcccgcccccttacgcgttgtctcattccaccaaattcagaatatttacacaatgccttcatgattttatttttctggaaattgaagtgtcaattgggttctcaatatttcatgactccaaggatgcattaaatatttatttgtggtaagagaagatacctgccgcggaggagggtggcataattatttttttttcaaaaagctattctggggccctggccccctggctcctgtcacagctcagcttgaactctgtagcctctcaaatgaagaaggtggctgttatttaggactctgtggaaagcaaatcacagtgctgtttttaatgcctgagaaatgcactttaggggtagacttataccttaagtgaaggagtgggggaggaagaagcattcttaagtagaagcattttaaaataaggcaggattgcttttaatgttaaactacaaaactgtacagcctattttagtgtggactattaaa

SEQ ID NO: 28 - DNA, 1591 bp, Homo sapiens
  ref <308>: DATABASE ACCESSION NUMBER: Genbank / CR608739
  ref <309>: DATABASE ENTRY DATE: 2004-07-22
  ref <313>: RELEVANT RESIDUES IN SEQ ID NO: (1)..(1591)
cagggttctggtttcccgaatcacttggcaaataaccagcaggtgggcagatgtcttttttgcgttcctacggacgcgtaggcaagttcgatgcgccggagtgaacagccagggtcccatctctcagccggctcccgggcggtgctagcagggaagagggacgcgccggcgaatgaaacactactagcccggaaagccgccctggaccttccacccgggcagcgagtctggcggcccctcgccgccgccccctccgaccagaacagactgcgctgcgagccccgggcttccgccgcagcgcgcagcaattggaggtgccagaggagaccagcagcagcacttgatttagctgggctgcgggaattccgacctgtacactttccagaagcctgatctctccaagtccgcgcccagggactcgagccccggcttccaggttgacccgagcgcgcccccttcgtgctctcggccgcgttcaccgcgcgaggcctccgggtcctgttaagcccgagcagcacaggccgctgcctcagcatccaccccgcagcccacgtgtggcaagccggggaaggggtggagtgaacggccggagaccacgtggagaaaggggccgctttggcccttccatctgggtgccgggagcccctaggccctccggccatggccgacagcggcgatgctggcagctccggcccctggtggaaatcgctcaccaacagcagaaagaaaagcaaggaagccgcagtgggggtgccgcctcccgcccagcccgctcccggggagcccacgccacctgcgccgcccagcccggactggaccagcagctcccgggagaaccagcaccccaatctcctcgggggcgccggcgagccccccaaaccagacaagttatacggggacaaatccggcagcagccgccgcaatttgaagatctcgcgctccggccgctttaaggagaagaggaaagtgcgcgccacgctgctcccggaggcgggcaggtccccggaggaggcaggctttcctggtgacccccacgaggacaagcagtagccccaatagcctgcgcgctccaggactgcctacccagcactaccccaaacccccagttccaaacccgagacttcaggcccgcccccttacgcgttgtctcattccaccaaattcagaatatttacacaatgccttcatgattttatttttctggaaattgaagtgtcaattgggttctcaatatttcatgactccaaggatgcattaaatatttatttgtggtaagagaagatacctgccgcggaggagggtggcataattatttttttttcaaaaagctattctggggccctggccccctggctcctgtcacagctcagcttgaactctgtagcctctcaaatgaagaaggtggctgttatttaggactctgtggaaagcaaatcacagtgctgtttttaatgcctgagaaatgcactttaggggtagacttataccttaagtgaaggagtgggggaggaagaagcattcttaagtagaagcattttaaaataaggcaggattgcttttaatgttaaactacaaaactgtacagcctatt

SEQ ID NO: 29 - DNA, 1587 bp, Homo sapiens
  ref <308>: DATABASE ACCESSION NUMBER: Genbank / CR610203
  ref <309>: DATABASE ENTRY DATE: 2004-07-22
  ref <313>: RELEVANT RESIDUES IN SEQ ID NO: (1)..(1587)
gggttctggtttcccgaatcacttggcaaataaccagcaggtgggcagatgtcttttttgcgttcctacggacgcgtaggcaagttcgatgcgccggagtgaacagccagggtcccatctctcagccggctcccgggcggtgctagcagggaagagggacgcgccggcgaatgaaacactactagcccggaaagccgccctggaccttccacccgggcagcgagtctggcggcccctcgccgccgccccctccgaccagaacagactgcgctgcgagccccgggcttccgccgcagcgcgcagcaattggaggtgccagaggagaccagcagcagcacttgatttagctgggctgcgggaattccgacctgtacactttccagaagcctgatctctccaagtccgcgcccagggactcgagccccggcttccaggttgacccgagcgcgcccccttcgtgctctcggccgcgttcaccgcgcgaggcctccgggtcctgttaagcccgagcagcacaggccgctgcctcagcatccaccccgcagcccacgtgtggcaagccggggaaggggtggagtgaacggccggagaccacgtggagaaaggggccgctttggcccttccatctgggtgccgggagcccctaggccctccggccatggccgacagcggcgatgctggcagctccggcccctggtggaaatcgctcaccaacagcagaaagaaaagcaaggaagccgcagtgggggtgccgcctcccgcccagcccgctcccggggagcccacgccacctgcgccgcccagcccggactggaccagcagctcccgggagaaccagcaccccaatctcctcgggggcgccggcgagccccccaaaccagacaagttatacggggacaaatccggcagcagccgccgcaatttgaagatctcgcgctccggccgctttaaggagaagaggaaagtgcgcgccacgctgctcccggaggcgggcaggtccccggaggaggcaggctttcctggtgacccccacgaggacaagcagtagccccaatagcctgcgcgctccaggactgcctacccagcactaccccaaacccccagttccaaacccgagacttcaggcccgcccccttacgcgttgtctcattccaccaaattcagaatatttacacaatgccttcatgattttatttttctggaaattgaagtgtcaattgggttctcaatatttcatgactccaaggatgcattaaatatttatttgtggtaagagaagatacctgccgcggaggagggtggcataattatttttttttcaaaaagctattctggggccctggccccctggctcctgtcacagctcagcttgaactctgtagcctctcaaatgaagaaggtggctgttatttaggactctgtggaaagcaaatcacagtgctgtttttaatgcctgagaaatgcactttaggggtagacttataccttaagtgaaggagtgggggaggaagaagcattcttaagtagaagcattttaaaataaggcaggattgcttttaatgttaaactacaaaactgtacagccta

SEQ ID NO: 30 - DNA, 1589 bp, Homo sapiens
  ref <308>: DATABASE ACCESSION NUMBER: Genbank / BC018144
  ref <309>: DATABASE ENTRY DATE: 2004-06-25
  ref <313>: RELEVANT RESIDUES IN SEQ ID NO: (1)..(1589)
tgtcttttttgcgttcctacggacgcgtaggcaagttcgatgcgccggagtgaacagccagggtcccatctctcagccggctcccgggcggtgctagcagggaagagggacgcgccggcgaatgaaacactactagcccggaaagccgccctggaccttccacccgggcagcgagtctggcggcccctcgccgccgccccctccgaccagaacagactgcgctgcgagccccgggcttccgccgcagcgcgcagcaattggaggtgccagaggagaccagcagcagcacttgatttagctgggctgcgggaattccgacctgtacactttccagaagcctgatctctccaagtccgcgcccagggactcgagccccggcttccaggttgacccgagcgcgcccccttcgtgctctcggccgcgttcaccgcgcgaggcctccgggtcctgttaagcccgagcagcacaggccgctgcctcagcatccaccccgcagcccacgtgtggcaagccggggaaggggtggagtgaacggccggagaccacgtggagaaaggggccgctttggcccttccatctgggtgccgggagcccctaggccctccggccatggccgacagcggcgatgctggcagctcgggcccctggtggaaatcgctcaccaacagcagaaagaaaagcaaggaagccgcagtgggggtgccgcctcccgcccagcccgctcccggggagcccacgccacctgcgccgcccagcccggactggaccagcagctcccgggagaaccagcaccccaatctcctcgggggcgccggcgagccccccaaaccagacaagttatacggggacaaatccggcagcagccgccgcaatttgaagatctcgcgctccggccgctttaaggagaagaggaaagtgcgcgccacgctgctcccggaggcgggcaggtcctcggaggaggcaggctttcctggtgacccccacgaggacaagcagtagccccaatagcctgcgcgctccaggactgcctacccagcactaccccaaacccccagttccaaacccgagacttcaggcccgcccccttacgcgttgtctcattccaccaaattcagaatatttacacaatgccttcatgattttatttttctggaaattgaagtgtcaattgggttctcaatatttcatgactccaaggatgcattaaatatttatttgtggtaagagaagatacctgccgcggaggagggtggcataattattttttttcaaaaagctattctggggccctggccccctggctcctgtcacagctcagcttgaactctgtagcctctcaaatgaagaaggtggctgttatttaggactctgtggaaagcaaatcacagtgctgtttttaatgcctgagaaatgcactttaggggtagacttataccttaagtgaaggagtgggggaggaagaagcattcttaagtagaagcattttaaaataaggcaggattgcttttaatgttaaactacaaaactgtacagcctattttagtgtggactattaaaacccttgccctgtaaaaaaaaaaaaaaaaaaa

SEQ ID NO: 31 - DNA, 1496 bp, Homo sapiens
  ref <308>: DATABASE ACCESSION NUMBER: Genbank / CR604389
  ref <309>: DATABASE ENTRY DATE: 2004-07-22
  ref <313>: RELEVANT RESIDUES IN SEQ ID NO: (1)..(1496)
ccggagtgaacagccagggtcccatctctcagccggctcccgggcggtgctagcagggaagagggacgcgccggcgaatgaaacactactagcccggaaagccgccctggaccttccacccgggcagcgagtctggcggcccctcgccgccgccccctccgaccagaacagactgcgctgcgagccccgggcttccgccgcagcgcgcagcaattggaggtgccagaggagaccagcagcagcacttgatttagctgggctgcgggaattccgacctgtacactttccagaagcctgatctctccaagtccgcgcccagggactcgagccccggcttccaggttgacccgagcgcgcccccttcgtgctctcggccgcgttcaccgcgcgaggcctccgggtcctgttaagcccgagcagcacaggccgctgcctcagcatccaccccgcagcccacgtgtggcaagccggggaaggggtggagtgaacggccggagaccacgtggagaaaggggccgctttggcccttccatctgggtgccgggagcccctaggccctccggccatggccgacagcggcgatgctggcagctccggcccctggtggaaatcgctcaccaacagcagaaagaaaagcaaggaagccgcagtgggggtgccgcctcccgcccagcccgctcccggggagcccacgccacctgcgccgcccagcccggactggaccagcagctcccgggagaaccagcaccccaatctcctcgggggcgccggcgagccccccaaaccagacaagttatacggggacaaatccggcagcagccgccgcaatttgaagatctcgcgctccggccgctttaaggagaagaggaaagtgcgcgccacgctgctcccggaggcgggcaggtccccggaggaggcaggctttcctggtgacccccacgaggacaagcagtagccccaatagcctgcgcgctccaggactgcctacccagcactaccccaaacccccagttccaaacccgagacttcaggcccgcccccttacgcgttgtctcattccaccaaattcagaatatttacacaatgccttcatgattttatttttctggaaattgaagtgtcaattgggttctcaatatttcatgactccaaggatgcattaaatatttatttgtggtaagagaagatacctgccgcggaggagggtggcataattatttttttttcaaaaagctattctggggccctggccccctggctcctgtcacagctcagcttgaactctgtagcctctcaaatgaagaaggtggctgttatttaggactctgtggaaagcaaatcacagtgctgtttttaatgcctgagaaatgcactttaggggtagacttataccttaagtgaaggagtgggggaggaagaagcattcttaagtagaagcattttaaaataaggcaggattgcttttaatgttaaactacaaaactgtacagcctatt

SEQ ID NO: 32 - DNA, 2193 bp, Homo sapiens
  ref <308>: DATABASE ACCESSION NUMBER: Genbanki / NM_017949
  ref <309>: DATABASE ENTRY DATE: 2005-09-23
  ref <313>: RELEVANT RESIDUES IN SEQ ID NO: (1)..(2193)
aagtgtggccacttcctcttgcctgtcctgactcaccctggctgctgcccattgtggctgccatggaaatacaactcgaggagaacccagaccaggacagggaggccatgttctgggtggcagtggagcctggcaaggtgccctcctggcctgggactgctgtggcagaggaagcatgaggtggggcctggcctgcccagctcagagctgaggtgcccccttggttttttacctccctgctccctggctggcccactgcacccacgcactcaacgcagtggcttcgctcctcctccaggatggctggaggtgggaccattgccaggatggcctcaagtgcccagccctcctggaagactcagtttccctctttgtcccatgcagaggtgcagagattcattattgtgggaccatccgtgaaaccggtgccaggatgcaccgggggtgtggatgaccggccgcccagccccagcacggcccggtcccgcatgccaccgccccgcgaggaccgctctccaagggctgcctggggccccgcgcccgtactctcctaacccggagctcttggcagcccattgctgcctggctacccggggaggaggaggcgcaggagtgagctgcccgagaccgcagggcaagtaagcggctgacggcggaaagaccctggggaaggggctttgcggccggctagaaacattttccccaagcggctccgcaaaatgaccagcctgttccgccggagcagcagcggcagcggcgggggtggcaccgccggggcacgcgggggcgggggaggcacggccgccccccaggagctcaacaacagccggcctgcccgccaggtgcgccgcctggagttcaaccaggccatggacgacttcaagaccatgttccccaacatggattacgacatcatcgaatgcgtgctgcgcgccaacagcggcgctgtggacgccaccatcgaccagctgctgcagatgaacctggagggcggtggcagcagcggcggcgtctatgaggacagctccgactcggaggacagcatccccccggagatcttggaaaggactttggaacctgatagctcggatgaagagcccccacctgtgtactccccgccagcctaccacatgcacgtgttcgaccgaccctaccctctggctcccccgactccgcctccccgtatcgacgcgctgggctctggagcccctacaagccagagacgctatcggaactggaacccaccactgctgggcaaccttccggatgactttctccgcatcctgccccagcagctggacagcatacagggtaacgctgggggccccaagcctgggagtggagagggatgtccacctgccatggctgggccagggcccggagaccaggagagccgctggaagcagtacctggaggacgagaggatcgcgcttttcctgcagaacgaggagttcatgaaggaactgcaacggaaccgcgacttcctcctcgctctggagagagatcgattgaaatacgaatcccagaaatctaaatccagcagcgtggctgtcggaaacgactttggcttttcctctcctgtcccaggaactggcgacgccaaccccgctgtgtctgaagatgccttattcagggacaagctgaaacacatgggaaagtccacccggaggaaactgtttgaacttgcccgagccttctcagagaagaccaaaatgaggaagtcaaagaggaaacacttgttgaagcatcagtcgctgggggctgccgcgtcaacagccaacctcctggatgatgtggagggccacgcgtgtgatgaagacttccggggcaggcgtcaggaggcacccaaggtggaggaaggcctgcgagaaggacagtaagagatgccagcagctcttccttgccagagatgatctgacccggtgggggcagctggaaagcaacactggcccccagctgaagggcccagctgcagccagacagatggtgcttgagaaccgaggcccggtgatcctccagccacagtccagcccaaccactgccactttccatgggacttagaacttcggagttgctgccttgcaattggaggaaggacctggggcccctataggcagcagccaattacagccccttttgtagccgggcgttcctatggtcaaagagtggaaatgcaggaaccaacctcccttt

SEQ ID NO: 33 - DNA, 2193 bp, Homo sapiens; identical to SEQ ID NO: 32
  ref <308>: DATABASE ACCESSION NUMBER: Genbank / AK000746
  ref <309>: DATABASE ENTRY DATE: 2003-09-15
  ref <313>: RELEVANT RESIDUES IN SEQ ID NO: (1)..(2193)

SEQ ID NO: 34 - DNA, 1903 bp, Homo sapiens
  ref <308>: DATABASE ACCESSION NUMBER: Genbank / BC056882
  ref <309>: DATABASE ENTRY DATE: 2004-03-25
  ref <313>: RELEVANT RESIDUES IN SEQ ID NO: (1)..(1903)
ggccccgccgccaccgctgcggattgcagcccctcccccgcccggcccgcggcccggcccctgcggcccgcgcggagcggacgcgcgtggcgctgcccggtgggaccatccgtgaaaccggtgccaggatgcaccgggggtgtggatgaccggccgcccagccccagcacggcccggtcccgcatgccaccgccccgcgaggaccgctctccaagggctgcctggggccccgcgcccgtactctcctaacccggagctcttggcagcccattgctgcctggctacccggggaggaggaggcgcaggagtgagctgcccgagaccgcagggcaagtaagcggctgacggcggaaagaccctggggaaggggctttgcggccggctagaaacattttccccaagcggctccgcaaaatgaccagcctgttccgccggagcagcagcggcagcggcgggggtggcaccgccggggcacgcgggggcgggggaggcacggccgccccccaggagctcaacaacagccggcctgcccgccaggtgcgccgcctggagttcaaccaggccatggacgacttcaagaccatgttccccaacatggattacgacatcatcgaatgcgtgctgcgcgccaacagcggcgctgtggacgccaccatcgaccagctgctgcagatgaacctggagggcggtggcagcagcggcggcgtctatgaggacagctccgactcggaggacagcatccccccggagatcttggaaaggactttggaacctgatagctcggatgaagagcccccacctgtgtactccccgccagcctaccacatgcacgtgttcgaccggccctaccctctggctcccccgactccgcctccccgtatcgacgcgctgggctctggagcccctacaagccagagacgctatcggaactggaacccaccactgctgggcaaccttccggatgactttctccgcatcctgccccagcagctggacagcatacagggtaacgctgggggccccaagcctgggagtggagagggatgtccacctgccatggctgggccagggcccggagaccaggagagccgctggaagcagtacctggaggacgagaggatcgcgcttttcctgcagaacgaggagttcatgaaggagctgcaacggaaccgcgacttcctcctcgctctggagagagatcgattgaaatacgaatcccagaaatctaaatccagcagcgtggctgtcggaaacgactttggcttttcctctcctgtcccaggaactggcgacgccaaccccgctgtgtctgaagatgccttattcagggacaagctgaaacacatgggaaagtccacccggaggaaactgtttgaacttgcccgagccttctcagagaagaccaaaatgaggaagtcaaagaggaaacacttgttgaagcatcagtcgctgggggctgccgcgtcaacagccaacctcctggatgatgtggagggccacgcgtgtgatgaagacttccggggcaggcgtcaggaggcacccaaggtggaggaaggcctgcgagaaggacagtaagagatgccagcagctcttccttgccagagatgatctgacccggtgggggcagctggaaagcaacactggcccccagctgaagggcccagctgcagccagacagatggtgcttgagaaccgaggcccggtgatcctccagccacagtccagcccaaccactgccactttccatgggacttagaacttcggagttgctgccttgcaattggaggaaggacctggggcccctataggcagcagccaattacagccccttttgtagccaggcgttcctatggtcaaagagtggaaatgcaggaaccaacctccctttaaaaaaaaaaaaaaa

SEQ ID NO: 35 - DNA, 1543 bp, Homo sapiens
  ref <308>: DATABASE ACCESSION NUMBER: Genbank / AK000977
  ref <309>: DATABASE ENTRY DATE: 2006-01-19
  ref <313>: RELEVANT RESIDUES IN SEQ ID NO: (1)..(1543)
tactggccattgctgcctggctacccggggaggaggaggcgcaggagtgagctgcccgagaccgcagggcaagtaagcggctgacggcggaaagaccctggggaaggggctttgcggccggctagaaacattttccccaagcggctccgcaaaatgaccagcctgttccgccggagcagcagcggcagcggcgggggtggcaccgccggggcacgcgggggcgggggaggcacggccgccccccaggagctcaacaacagccggcctgcccgccaggtgcgccgcctggagctcaaccaggccatggacgacttcaagaccatgttccccaacatggattacgacatcatcgaatgcgtgctgcgcgccaacagcggcgctgtggacgccaccatcgaccagctgctgcagatgaacctggagggcggtggcagcagcggcggcgtctatgaggacagctccgactcggaggacagcatccccccggagatcttggaaaggactttggaacctgatagctcggatgaagagcccccacctgtgtactccccgccagcctaccacatgcacgtgttcgaccggccctaccctctggctcccccgactccgcctccccgtatcgacgcgctgggctctggagcccctacaagccagagacgctatcggaactggaacccaccactgctgggcaaccttccggatgactttctccgcatcctgccccagcagctggacagcatacagggtaacgctgggggccccaagcctgggagtggagagggatgtccacctgccatggctgggccagggcccggagaccaggagagccgctggaagcagtacctggagagagatcgattgaaatacgaatcccagaaatctaaatccagcagcgtggctgtcggaaacgactttggcttttcctctcctgtcccaggaactggcgacgccaaccccgctgtgtctgaagatgccttattcagggacaagctgaaacacatgggaaagtccacccggaggaaactgtttgaacttgcccgagccttctcagagaagaccaaaatgaggaagtcaaagaggaaacacttgttgaagcatcagtcgctgggggctgccgcgtcaacagccaacctcctggatgatgtggagggccacgcgtgtgatgaagacttccggggcaggcgtcaggaggcacccaaggtggaggaaggcctgcgagaaggacagtaagagatgccagcagctcttccttgccagagatgatctgacccggtgggggcagctggaaagcaacactggcccccagctgaagggcccagctgcagccagacagatggtgcttgagaaccgaggcccggtgatcctccagccacagtccagcccaaccactgccactttccatgggacttagaacttcggagttgctgccttgcaattggaggaaggacctggggcccctataggcagcagccaattacagccccttttgtagccaggcgttcctatggtcaaagagtggaaatgcaggagccaacctcccttt

SEQ ID NO: 36 - DNA, 921 bp, Homo sapiens
  ref <308>: DATABASE ACCESSION NUMBER: Genbank / NM_006432
  ref <309>: DATABASE ENTRY DATE: 2006-04-16
  ref <313>: RELEVANT RESIDUES IN SEQ ID NO: (1)..(921)
gacaggtcgcctgactgggctcctccccgggcccgccccgacaggtttgtcttgtgaccgcgggcggccgctgcttctttcccgagcttggaacttcgttatccgcgatgcgtttcctggcagctacattcctgctcctggcgctcagcaccgctgcccaggccgaaccggtgcagttcaaggactgcggttctgtggatggagttataaaggaagtgaatgtgagcccatgccccacccaaccctgccagctgagcaaaggacagtcttacagcgtcaatgtcaccttcaccagcaatattcagtctaaaagcagcaaggccgtggtgcatggcatcctgatgggcgtcccagttccctttcccattcctgagcctgatggttgtaagagtggaattaactgccctatccaaaaagacaagacctatagctacctgaataaactaccagtgaaaagcgaatatccctctataaaactggtggtggagtggcaacttcaggatgacaaaaaccaaagtctcttctgctgggaaatcccagtacagatcgtttctcatctctaagtgcctcattgagttcggtgcatctggccaatgagtctgctgagactcttgacagcacctccagctctgctgcttcaacaacagtgacttgctctccaatggtatccagtgattcgttgaagaggaggtgctctgtagcagaaactgagctccgggtggctggttctcagtggttgtctcatgtctctttttctgtcttaggtggtttcattaaatgcagcacttggttagcagatgtttaattttttttttaacaacattaacttgtggcctctttctacacctggaaatttactcttgaataaataaaaactcgtttgtcttgtcttctgcaaaaaaaaaaaaaaaaaaaaaaaaa

SEQ ID NO: 37 - DNA, 920 bp, Homo sapiens
  ref <308>: DATABASE ACCESSION NUMBER: Genbank / BC002532
  ref <309>: DATABASE ENTRY DATE: 2005-03-08
  ref <313>: RELEVANT RESIDUES IN SEQ ID NO: (1)..(920)
acaggtcgcctgactgggctcctccccgggcccgccccgacaggtttgtcttgtgaccgcgggcggccgctgcttctttcccgagcttggaacttcgttatccgcgatgcgtttcctggcagctacattcctgctcctggcgctcagcaccgctgcccaggccgaaccggtgcagttcaaggactgcggttctgtggatggagttataaaggaagtgaatgtgagcccatgccccacccaaccctgccagctgagcaaaggacagtcttacagcgtcaatgtcaccttcaccagcaatattcagtctaaaagcagcaaggccgtggtgcatggcatcctgatgggcgtcccagttccctttcccattcctgagcctgatggttgtaagagtggaattaactgccctatccaaaaagacaagacctatagctacctgaataaactaccagtgaaaagcgaatatccctctataaaactggtggtggagtggcaacttcaggatgacaaaaaccaaagtctcttctgctgggaaatcccagtacagatcgtttctcatctctaagtgcctcattgagttcggtgcatctggccaatgagtctgctgagactcttgacagcacctccagctctgctgcttcaacaacagtgacttgctctccaatggtatccagtgattcgttgaagaggaggtgctctgtagcagaaactgagctccgggtggctggttctcagtggttgtctcatgtctctttttctgtcttaggtggtttcattaaatgcagcacttggttagcagatgtttaattttttttttaacaacattaacttgtggcctctttctacacctggaaatttactcttgaataaataaaaactcgtttgtcttgtcaaaaaaaaaaaaaaaaaaaaaaaaaaaaaaa

SEQ ID NO: 38 - DNA, 818 bp, Homo sapiens
  ref <308>: DATABASE ACCESSION NUMBER: Genbank / CR609490
  ref <309>: DATABASE ENTRY DATE: 2004-07-22
  ref <313>: RELEVANT RESIDUES IN SEQ ID NO: (1)..(818)
tgcttctttcccgagcttggaacttcgttatccgcgatgcgtttcctggcagctacattcctgctcctggcgctcagcaccgctgcccaggccgaaccggtgcagttcaaggactgcggttctgtggatggagttataaaggaagtgaatgtgagcccatgccccacccaaccctgccagctgagcaaaggacagtcttacagcgtcaatgtcaccttcaccagcaatattcagtctaaaagcagcaaggccgtggtgcatggcatcctgatgggcgtcccagttccctttcccattcctgagcctgatggttgtaagagtggaattaactgccctatccaaaaagacaagacctatagctacctgaataaactaccagtgaaaagcgaatatccctctataaaactggtggtggagtggcaacttcaggatgacaaaaaccaaagtctcttctgctgggaaatcccagtacagatcgtttctcatctctaagtgcctcattgagttcggtgcatctggccaatgagtctgctgagactcttgacagcacctccagctctgctgcttcaacaacagtgacttgctctccaatggtatccagtgattcgttgaagaggaggtgctctgtagcagaaactgagctccgggtggctggttctcagtggttgtctcatgtctctttttctgtcttaggtggtttcattaaatgcagcacttggttagcagatgtttaattttttttttaacaacattaacttgtggcctctttctacacctggaaatttactcttgaataaataaaaactcgtttgtcttgt

SEQ ID NO: 39 - DNA, 811 bp, Homo sapiens
  ref <308>: DATABASE ACCESSION NUMBER: Genbank / CR608935
  ref <309>: DATABASE ENTRY DATE: 2004-07-22
  ref <313>: RELEVANT RESIDUES IN SEQ ID NO: (1)..(811)
ttcccgagcttggaacttcgttatccgcgatgcgtttcctggcagctacattcctgctcctggcgctcagcaccgctgcccaggccgaaccggtgcagttcaaggactgcggttctgtggatggagttataaaggaagtgaatgtgagcccatgccccacccaaccctgccagctgagcaaaggacagtcttacagcgtcaatgtcaccttcaccagcaatattcagtctaaaagcagcaaggccgtggtgcatggcatcctgatgggcgtcccagttccctttcccattcctgagcctgatggttgtaagagtggaattaactgccctatccaaaaagacaagacctatagctacctgaataaactaccagtgaaaagcgaatatccctctataaaactggtggtggagtggcaacttcaggatgacaaaaaccaaagtctcttctgctgggaaatcccagtacagatcgtttctcatctctaagtgcctcattgagttcggtgcatctggccaatgagtctgctgagactcttgacagcacctccagctctgctgcttcaacaacagtgacttgctctccaatggtatccagtgattcgttgaagaggaggtgctctgtagcagaaactgagctccgggtggctggttctcagtggttgtctcatgtctctttttctgtcttaggtggtttcattaaatgcagcacttggttagcagatgtttaattttttttttaacaacattaacttgtggcctctttctacacctggaaatttactcttgaataaataaaaactcgtttgtcttgt

SEQ ID NO: 40 - DNA, 806 bp, Homo sapiens
  ref <308>: DATABASE ACCESSION NUMBER: Genbank / CR605546
  ref <309>: DATABASE ENTRY DATE: 2004-07-22
  ref <313>: RELEVANT RESIDUES IN SEQ ID NO: (1)..(806)
ggaacttcgttatccgcgatgcgtttcctggcagctacattcctgctcctggcgctcagcaccgctgcccaggccgaaccggtgcagttcaaggactgcggttctgtggatggagttataaaggaagtgaatgtgagcccatgccccacccaaccctgccagctgagcaaaggacagtcttacagcgtcaatgtcaccttcaccagcaatattcagtctaaaagcagcaaggccgtggtgcatggcatcctgatgggcgtcccagttccctttcccattcctgagcctgatggttgtaagagtggaattaactgccctatccaaaaagacaagacctatagctacctgaataaactaccagtgaaaagcgaatatccctctataaaactggtggtggagtggcaacttcaggatgacaaaaaccaaagtctcttctgctgggaaatcccagtacagatcgtttctcatctctaagtgcctcattgagttcggtgcatctggccaatgagtctgctgagactcttgacagcacctccagctctgctgcttcaacaacagtgacttgctctccaatggtatccagtgattcgttgaagaggaggtgctctgtagcagaaactgagctccgggtggctggttctcagtggttgtctcatgtctctttttctgtcttaggtggtttcattaaatgcagcacttggttagcagatgtttaattttttttttaacaacattaacttgtggcctctttctacacctggaaatttactcttgaataaataaaaactcgtttgtcttgtcttctg

SEQ ID NO: 41 - DNA, 793 bp, Homo sapiens
  ref <308>: DATABASE ACCESSION NUMBER: Genbank / CR622486
  ref <309>: DATABASE ENTRY DATE: 2004-07-22
  ref <313>: RELEVANT RESIDUES IN SEQ ID NO: (1)..(793)
gcgatgcgtttcctggcagctacattcctgctcctggcgctcagcaccgctgcccaggccgaaccggtgcagttcaaggactgcggttctgtggatggagttataaaggaagtgaatgtgagcccatgccccacccaaccctgccagctgagcaaaggacagtcttacagcgtcaatgtcaccttcaccagcaatattcagtctaaaagcagcaaggccgtggtgcatggcatcctgatgggcgtcccagttccctttcccattcctgagcctgatggttgtaagagtggaattaactgccctatccaaaaagacaagacctatagctacctgaataaactaccagtgaaaagcgaatatccctctataaaactggtggtggagtggcaacttcaggatgacaaaaaccaaagtctcttctgctgggaaatcccagtacagatcgtttctcatctctaagtgcctcattgagttcggtgcatctggccaatgagtctgctgagactcttgacagcacctccagctctgctgcttcaacaacagtgacttgctctccaatggtatccagtgattcgttgaagaggaggtgctctgtagcagaaactgagctccgggtggctggttctcagtggttgtctcatgtctctttttctgtcttaggtggtttcattaaatgcagcacttggttagcagatgtttaattttttttttaacaacattaacttgtggcctctttctacacctggaaatttactcttgaataaataaaaactcgtttgtcttgtcttctgca

SEQ ID NO: 42 - DNA, 789 bp, Homo sapiens
  ref <308>: DATABASE ACCESSION NUMBER: Genbank / AK222474
  ref <309>: DATABASE ENTRY DATE: 2005-04-25
  ref <313>: RELEVANT RESIDUES IN SEQ ID NO: (1)..(789)
cccgagcttggaacttcgttatccgcgatgcgtttcctggcagctacattcctgctcctggcgctcagcaccgctgcccaggccgaaccggtgcagttcagggactgcggttctgtggatggagttataaaggaagtgaatgtgagcccatgccccacccaaccctgccagctgagcaaaggacagtcttacagcgtcaatgtcaccttcaccagcaatgttcagtctaagagcagcaaggccgtggtgcatggcatcctgatgggcgtcccagttccctttcccattcctgagcctgatggttgtaagagtggaattaactgccctatccaaaaagacaagacctatagctacctgaataaactaccagtgaaaagcgaatatccctctataaaactggtggtggagtggcaacttcaggatgacaaaaaccaaagtctcttctgctgggaaatcccagtacagatcgtttctcatctctaagtgcctcattgagttcggtgcatctggccaatgagtctgctgagactcttgacagcacctccagctctgctgcttcaacaacagtgacttgctctccaatggtatccagtgattcgttgaagaggaggtgctctgtagcagaaactgagctccgggtggctggttctcagtggttgtctcatgtctctttttctgtcttaggtggtttcattaaatgcagcacttggttagcagatgtttaattttttttttaacaacattaacttgtggcctctttctacacctggaaatttactcttgaataaa

SEQ ID NO: 43 - DNA, 783 bp, Homo sapiens
  ref <308>: DATABASE ACCESSION NUMBER: Genbank / CR624497
  ref <309>: DATABASE ENTRY DATE: 2004-07-22
  ref <313>: RELEVANT RESIDUES IN SEQ ID NO: (1)..(783)
tcctggcagctacattcctgctcctggcgctcagcaccgctgcccaggccgaaccggtgcagttcaaggactgcggttctgtggatggagttataaaggaagtgaatgtgagcccatgccccacccaaccctgccagctgagcaaaggacagtcttacagcgtcaatgtcaccttcaccagcaatattcagtctaaaagcagcaaggccgtggtgcatggcatcctgatgggcgtcccagttccctttcccattcctgagcctgatggttgtaagagtggaattaactgccctatccaaaaagacaagacctatagctacctgaataaactaccagtgaaaagcgaatatccctctataaaactggtggtggagtggcaacttcaggatgacaaaaaccaaagtctcttctgctgggaaatcccagtacagatcgtttctcatctctaagtgcctcattgagttcggtgcatctggccaatgagtctgctgagactcttgacagcacctccagctctgctgcttcaacaacagtgacttgctctccaatggtatccagtgattcgttgaagaggaggtgctctgtagcagaaactgagctccgggtggctggttctcagtggttgtctcatgtctctttttctgtcttaggtggtttcattaaatgcagcacttggttagcagatgtttaattttttttttaacaacattaacttgtggcctctttctacacctggaaatttactcttgaataaataaaaactcgtttgtcttgtcttctgca

SEQ ID NO: 44 - DNA, 774 bp, Homo sapiens
  ref <308>: DATABASE ACCESSION NUMBER: Genbank / CR601885
  ref <309>: DATABASE ENTRY DATE: 2004-07-22
  ref <313>: RELEVANT RESIDUES IN SEQ ID NO: (1)..(774)
ctggcagctacattcctgctcctggcgctcagcaccgctgcccaggccgaaccggtgcagttcaaggactgcggttctgtggatggagttataaaggaagtgaatgtgagcccatgccccacccaaccctgccagctgagcaaaggacagtcttacagcgtcaatgtcaccttcaccagcaatattcagtctaaaagcagcaaggccgtggtgcatggcatcctgatgggcgtcccagttccctttcccattcctgagcctgatggttgtaagagtggaattaactgccctatccaaaaagacaagacctatagctacctgaataaactaccagtgaaaagcgaatatccctctataaaactggtggtggagtggcaacttcaggatgacaaaaaccaaagtctcttctgctgggaaatcccagtacagatcgtttctcatctctaagtgcctcattgagttcggtgcatctggccaatgagtctgctgagactcttgacagcacctccagctctgctgcttcaacaacagtgacttgctctccaatggtatccagtgattcgttgaagaggaggtgctctgtagcagaaactgagctccgggtggctggttctcagtggttgtctcatgtctctttttctgtcttaggtggtttcattaaatgcagcacttggttagcagatgtttaattttttttttaacaacattaacttgtggcctctttctacacctggaaatttactcttgaataaataaaaactcgtttgtcttgtc

SEQ ID NO: 45 - DNA, 808 bp, Homo sapiens
  ref <308>: DATABASE ACCESSION NUMBER: Genbank / X67698
  ref <309>: DATABASE ENTRY DATE: 2003-08-05
  ref <313>: RELEVANT RESIDUES IN SEQ ID NO: (1)..(808)
cggattccggatgcgtttcctggcagctacattcctgctcctggcgctcagcaccgctgcccaggccgaaccggtgcagttcaaggactgcggttctgtggatggagttataaaggaagtgaatgtgagcccatgccccacccaaccctgccagctgagcaaaggacagtcttacagcgtcaatgtcaccttcaccagcaatattcagtctaaaagcagcaaggccgtggtgcatggcatcctgatgggcgtcccagttccctttcccattcctgagcctgatggttgtaagagtggaattaactgccctatccaaaaagacaagacctatagctacctgaataaactaccagtgaaaagcgaatatccctctataaaactggtggtggagtggcaacttcaggatgacaaaaaccaaagtctcttctgctgggaaatcccagtacagatcgtttctcatctctaagtgcctcattgagttcggtgcatctggccaatgagtctgctgagactcttgacagcacctccagctctgctgcttcaacaacagtgacttgctctccaatggtatccagtgattcgttgaagaggaggtgctctgtagcagaaactgagctccgggtggctggttctcagtggttgtctcatgtctctttttctgtcttaggtggtttcattaaatgcagcacttggttagcagatgtttaatttttttttttaacaacattaacttgtggcctctttctacacctggaaatttactcttgaataaataaaaactcgtttgtcttgtaaaaaaaaaaaaaaa

SEQ ID NO: 46 - DNA, 491 bp, Homo sapiens
  ref <308>: DATABASE ACCESSION NUMBER: Genbank / CR595914
  ref <309>: DATABASE ENTRY DATE: 2004-07-21
  ref <313>: RELEVANT RESIDUES IN SEQ ID NO: (1)..(491)
tctaaaagcagcaaggccgtggtgcatggcatcctgatgggcgtcccagttccctttcccattcctgagcctgatggttgtaagagtggaattaactgccctatccaaaaagacaagacctatagctacctgaataaactaccagtgaaaagcgaatatccctctataaaactggtggtggagtggcaacttcaggatgacaaaaaccaaagtctcttctgctgggaaatcccagtacagatcgtttctcatctctaagtgcctcattgagttcggtgcatctggccaatgagtctgctgagactcttgacagcacctccagctctgctgcttcaacaacagtgacttgctctccaatggtatccagtgattcgttgaagaggaggtgctctgtagcagaaactgagctccgggtggctggttctcagtggttgtctcatgtctctttttctgtcttaggtggtttcattaaatgcagcacttggttagca

SEQ ID NO: 47 - DNA, 712 bp, Homo sapiens
  ref <308>: DATABASE ACCESSION NUMBER: Genbank / S70284
  ref <309>: DATABASE ENTRY DATE: 2005-06-04
  ref <313>: RELEVANT RESIDUES IN SEQ ID NO: (1)..(712)
ggcccacatgctccaagaggatatctcaagctcctataccaccaccaccaccattacagcgcctcctccaggggtcctgcagaatggaggagataagttggagacgatgcccctctacttggaagacgacattcgccctgatataaaagatgatatatacgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacccggcttgctgatgatgtgcttcatcctgcccacgcttgtgccctggtactgctggggc

SEQ ID NO: 48 - DNA, 1470 bp, Homo sapiens
  ref <308>: DATABASE ACCESSION NUMBER: Genbank / Y13647
  ref <309>: DATABASE ENTRY DATE: 2003-08-05
  ref <313>: RELEVANT RESIDUES IN SEQ ID NO: (1)..(1470)
gacggtcacccgttgccagctctagcctttaaattcccggctcggggacctccacgcaccgcggctagcgccgacaaccagctagcgtgcaaggcgccgcggctcagcgcgtaccggcgggtttcgaaaccgcagtcctccggcgaccccgaactccgctccggagcctcagccccctggaaagtgatcccggcatcggagagccaagatgccggcccacttgctgcaggacgatatctctagctcctataccaccaccaccaccattacagcgcctcctccaggggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgatgatgtgcttcatcctgcccacgcttgtgccctggtatttctggggtgaaacttttcaaaacagtgtgttcgttgccactttcttgcgatatgctgtggtgcttaatgccacctggctggtgaacagtgctgcccacctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaacacattcttcattgattggatggccgccctcggtctgacctatgaccggaagaaagtctccaaggccgccatcttggccaggattaaaagaaccggagatggaaactacaagagtggctgagtttggggtccctcaggttcctttttcaaaaaccagccaggcagaggttttaatgtctgtttattaactactgaataatgctaccaggatgctaaagatgatgatgttaacccattccagtacagtattcttttaaaattcaaaagtattgaaagccaaaaaaaaaaaaaaaaaaaaaaaaa

SEQ ID NO: 49 - DNA, 5473 bp, Homo sapiens
  ref <308>: DATABASE ACCESSION NUMBER: Genbank / NM_005063
  ref <309>: DATABASE ENTRY DATE: 2006-04-16
  ref <313>: RELEVANT RESIDUES IN SEQ ID NO: (1)..(5473)
ggcaggacgaggtggcaccaaattcccttcggccaatgacgagccggagtttacagaagcctcattagcatttccccagaggcaggggcaggggcagaggccgggtggtgtggtgtcggtgtcggcagcatccccggcgccctgctgcggtcgccgcgagcctcggcctctgtctcctccccctcccgcccttacctccacgcgggaccgcccgcgccagtcaactcctcgcactttgcccctgcttggcagcggataaaagggggctgaggaaataccggacacggtcacccgttgccagctctagcctttaaattcccggctcggggacctccacgcaccgcggctagcgccgacaaccagctagcgtgcaaggcgccgcggctcagcgcgtaccggcgggcttcgaaaccgcagtcctccggcgaccccgaactccgctccggagcctcagccccctggaaagtgatcccggcatccgagagccaagatgccggcccacttgctgcaggacgatatctctagctcctataccaccaccaccaccattacagcgcctccctccagggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgatgatgtgcttcatcctgcccacgcttgtgccctggtatttctggggtgaaacttttcaaaacagtgtgttcgttgccactttcttgcgatatgctgtggtgcttaatgccacctggctggtgaacagtgctgcccacctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaccacattcttcattgattgcatggccgccctcggtctggcctatgaccggaagaaagtctccaaggccgccatcttggccaggattaaaagaaccggagatggaaactacaagagtggctgagtttggggtccctcaggttcctttttcaaaaaccagccaggcagaggttttaatgtctgtttattaactactgaataatgctaccaggatgctaaagatgatgatgttaacccattccagtacagtattcttttaaaattcaaaagtattgaaagccaacaactctgcctttatgatgctaagctgatattatttcttctcttatcctctctctcttctaggcccattgtcctccttttcactttattgctatcgccctcctttcccttattgcctcccaggcaagcagctggtcagtctttgctcagtgtccagcttccaaagcctagacaacctttctgtagcctaaaacgaatggtctttgctccagataactctctttccttgagctgttgtgagctttgaagtaggtggcttgagctagagataaaacagaatcttctgggtagtcccctgttgattatcttcagcccaggcttttgctagatggaatggaaaagcaacttcatttgacacaaagcttctaaagcaggtaaattgtcgggggagagagttagcatgtatgaatgtaaggatgagggaagcgaagcaagaggaacctctcgccatgatcagacatacagctgcctacctaatgaggacttcaagccccaccacatagcatgcttcctttctctcctggctcggggtaaaaagtggctgcggtgtttggcaatgctaattcaatgccgcaacatatagttgaggccgaggataaagaaaagacattttaagtttgtagtaaaagtggtctctgctggggaagggttttcttttctttttttctttaataacaaggagatttcttagttcatatatcaagaagtcttgaagttgggtgtttccagaattggtaaaaacagcagctcatagaattttgagtattccatgagctgctcattacagttctttcctctttctgctctgccatcttcaggatattggttcttcccctcatagtaataagatggctgtggcatttccaaacatccaaaaaaagggaaggatttaaggaggtgaagtcgggtcaaaaataaaatatatatacatatatacattgcttagaacgttaaactattagagtatttcccttccaaagagggatgtttggaaaaaactctgaaggagaggaggaattagttgggatgccaatttcctctccactgctggacatgagatggagaggctgagggacaggatctataggcagcttctaagagcgaacttcacataggaagggatctgagaacacgttgccaggggcttgagaaggttactgagtgagttattgggagtcttaataaaataaactagatattaggtccattcattaattagttccagtttctccttgaaatgagtaaaaactagaaggcttctctccacagtgttgtgccccttcactcatttttttttgaggagaagggggtctctgttaacatctagcctaaagtatacaactgcctggggggcagggttaggaatctcttcactaccctgattcttgattcctggctctaccctgtctgtcccttttctttgaccagatctttctcttccctgaacgttttcttctttccctggacaggcagcctcctttgtgtgtattcagaggcagtgatgacttgctgtccaggcagctccctcctgcacacagaatgctcagggtcactgaaccactgcttctcttttgaaagtagagctagctgccactttcacgtggcctccgcagtgtctccacctacacccctgtgctcccctgccacactgatggctcaagacaaggctggcaaaccctcccagaaacatctctggcccagaaagcctctctctccctccctctctcatgaggcacagccaagccaagcgctcatgttgagccagtgggccagccacagagcaaaagagggtttattttcagtcccctctctctgggtcagaaccagagggcatgctgaatgccccctgcttacttggtgagggtgccccgcctgagtcagtgctctcagctggcagtgcaatgcttgtagaagtaggaggaaacagttctcactgggaagaagcaagggcaagaacccaagtgcctcacctcgaaaggaggccctgttccctggagtcagggtgaactgcaaagctttggctgagacctgggatttgagataccacaaaccctgctgaacacagtgtctgttcagcaaactaaccagcattccctacagcctagggcagacaatagtatagaagtctggaaaaaaacaaaaacagaatttgagaaccttggaccactcctgtccctgtagctcagtcatcaaagcagaagtctggctttgctctattaagattggaaatgtacactaccaaacactcagtccactgttgagccccagtgctggaagggaggaaggcctttcttctgtgttaattgcgtagaggctacaggggttagcctggactaaaggcatccttgtcttttgagctattcacctcagtagaaaaggatctaagggaagatcactgtagtttagttctgttgacctgtgcacctaccccttggaaatgtctgctggtatttctaattccacaggtcatcagatgcctgcttgataatatataaacaataaaaacaactttcacttcttcctattgtaatcgtgtgccatggatctgatctgtaccatgaccctacataaggctggatggcacctcaggctgagggccccaatgtatgtgtggctgtgggtgtgggtgggagtgtgtctgctgagtaaggaacacgattttcaagattctaaagctcaattcaagtgacacattaatgataaactcagatctgatcaagagtccggatttctaacagtccttgctttggggggtgtgctgacaacttagctcaggtgccttacatcttttctaatcacagtgttgcatatgagcctgccctcactccctctgcagaatccctttgcacctgagaccctactgaagtggctggtagaaaaaggggcctgagtggaggattatcagtatcacgatttgcaggattcccttctgggcttcattctggaaacttttgttagggctgcttttcttaagtgcccacatttgatggagggtggaaataatttgaatgtatttgatttataagttttttttttttttttgggttaaaagatggttgtagcatttaaaatggaaaattttctccttggtttgctagtatcttgggtgtattctctgtaagtgtagctcaaataggtcatcatgaaaggttaaaaaagcgaggtggccatgttatgctggtggttaaggccagggcctctccaaccactgtgccactgacttgctgtgtgaccctgggcaagtcacttaactataaggtgcctcagttttccttctgttaaaatggggataataatactgacctacctcaaagggcagttttgaggcatgactaatgctttttagaaagcattttgggatccttcagcacaggaattctcaagacctgagtattttttataataggaatgtccaccatgaacttgatacgtccgtgtgtcccagatgctgtcattagtctatatggttctccaagaaactgaatgaatccattggagaagcggtggataactagccagacaaaatttgagaatacataaacaacgcattgccacggaaacatacagaggatgccttttctgtgattgggtgggattttttccctttttatgtgggatatagtagttacttgtgacaagaataattttggaataatttctattaatatcaactctgaagctaattgtactaatctgagattgtgtttgttcataataaaagtgaagtgaatctgattgcaaaaaa

SEQ ID NO: 50 - DNA, 1314 bp, Homo sapiens
  ref <308>: DATABASE ACCESSION NUMBER: Genbank / BC005807
  ref <309>: DATABASE ENTRY DATE: 2003-10-03
  ref <313>: RELEVANT RESIDUES IN SEQ ID NO: (1)..(1314)
aattcccggctcggggacctccacgcaccgcggctagcgccgacaaccagctagcgtgcaaggcgccgcggctcagcgcgtaccggcgggcttcgaaaccgcagtcctccggcgaccccgaactccgctccggagcctcagccccctggaaagtgatcccggcatccgagagccaagatgccggcccacttgctgcaggacgatatctctagctcctataccaccaccaccaccattacagcgcctccctccagggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgatgatgtgcttcatcctgcccacgcttgtgccctggtatttctggggtgaaacttttcaaaacagtgtgttcgttgccactttcttgcgatatgctgtggtgcttaatgccacctggctggtgaacagtgctgcccacctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaccacattcttcattgattgcatggccgccctcggtctggcctatgaccggaagaaagtctccaaggccgccatcttggccaggattaaaagaaccggagatggaaactaaaaaaaaaaaaaaaaaaaaaaaaaaaaaaaaaaaaaaaaaaaaaaaaaaaataaaaaaaaaaaaaaaaaa

SEQ ID NO: 51 - DNA, 1290 bp, Homo sapiens
  ref <308>: DATABASE ACCESSION NUMBER: Genbank / AK222862
  ref <309>: DATABASE ENTRY DATE: 2005-04-25
  ref <313>: RELEVANT RESIDUES IN SEQ ID NO: (1)..(1290)
acggtcacccgttgccagctctagcctttaaattcccggctcggggacctccacgcaccgcggctagcgccgacaaccagctagcgtgcaaggcgccgcggctcagcgcgtaccggcgggcttcgaaactgcagtcctccggcgaccccgaactccgctccggagcctcagccccctggaaagtgatcccggcatccgagagccaagatgccggcccacttgctgcaggacgatatctctagctcctataccaccaccaccaccattacagcgcctccctccagggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgatgatgtgcttcatcctgcccacgcttgtgccctggtatttctggggtgaaacttttcaaaacagtgtgttcgttgccactttcttgcgatatgctgtggtgcttaatgccacctggctggtgaacagtgctgccctcctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaccacattcttcattgattgcatggccgccctcggtctggcctatgaccggaagaaagtctccaaggccgccatcttggccaggattaaaagaaccggagatggaaactacaagagtggctgagtt

SEQ ID NO: 52 - DNA, 2214 bp, Homo sapiens
  ref <308>: DATABASE ACCESSION NUMBER: Genabnk / AB208982
  ref <309>: DATABASE ENTRY DATE: 2006-03-31
  ref <313>: RELEVANT RESIDUES IN SEQ ID NO: (1)..(2214)
gcttggcagcggataaaagggggctgaggaaataccggacacggtcacccgttgccagctctagcctttaaattcccggctcggggacctccacgcaccgcggctagcgccgacaaccagctagcgtgcaaggcgccgcggctcagcgcgtaccggcgggcttcgaaaccgcagtcctccggcgaccccgaactccgctccggagcctcagccccctggaaagtgatcccggcatccgagagccaagatgccggcccacttgctgcaggacgatatctctagctcctataccaccaccaccaccattacagcgcctccctccagggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgctgatgtgcttcatcctgcccacgcttgtgccctggtatttctggggtgaaacttttcaaaacagtgtgttcgttgccactttcttgcgatatgctgtggtgcttaatgccacctggctggtgaacagtgctgcccacctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaccacattcttcattgattgcatggccgccctcggtctggcctatgaccggaagaaagtctccaaggccgccatcttggccaggattaaaagaaccggagatggaaactacaagagtggctgagtttggggtccctcaggttcctttttcaaaaaccagccaggcagaggttttaatgtctgtttattaactactgaataatgctaccaggatgctaaagatgatgatgttaacccattccagtacagtattcttttaaaattcaaaagtattgaaagccaacaactctgcctttatgatgctaagctgatattatttcttctcttatcctctctctcttctaggcccattgtcctccttttcactttattgctatcgccctcctttcccttattgcctcccaggcaagcagctggtcagtctttgctcagtgtccagcttccaaagcctagacaacctttctgtagcctaaaacgaatggtctttgctccagataactctctttccttgagctgttgtgagctttgaagtaggtggcttgagctagagataaaacagaatcttctgggtagtcccctgttgattatcttcagcccaggcttttgctagatggaatggaaaagcaacttcatttgacacaaagcttctaaagcaggtaaattgtcgggggagagagttagcatgtatgaatgtaaggatgagggaagcgaagcaagaggaacctctcgccatgatcagacatacagctgcctacctaatgaggacttcaagccccaccacatagcatgcttcctttctctcctggctcggggtaaaaagtggctgcggtgtttggcaatgctaattcaatgccgcaacatatagttgaggccgaggataaagaaaagacattttaagtttgtagtaaaagtggtctctgctggggaagggttttcttttctttttttctttaataacaaggagatttcttagttcatatatcaagaagtcttgaagttgggtgtttccag

SEQ ID NO: 53 - DNA, 1631 bp, Homo sapiens
  ref <308>: DATABASE ACCESSION NUMBER: Genbank / BC062303
  ref <309>: DATABASE ENTRY DATE: 2005-03-08
  ref <313>: RELEVANT RESIDUES IN SEQ ID NO: (1)..(1631)
gtcggtgtcggcagcatccccggcgccctgctgcggtcgccgcgagcctcggcctctgtctcctccccctcccgcccttacctccacgcgggaccgcccgcgccagtcaactcctcgcactttgcccctgcttggcagcggataaaagggggctgaggaaataccggacacggtcacccgttgccagctctagcctttaaattcccggctcggggacctccacgcaccgcggctagcgccgacaaccagctagcgtgcaaggcgccgcggctcagcgcgtaccggcgggcttcgaaaccgcagtcctccggcgaccccgaactccgctccggagcctcagccccctggaaagtgatcccggcatcggagagccaagatgccggcccacttgctgcaggacgatatctctagctcctataccaccaccaccaccattacagcgcctccctccagggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgctgatgtgcttcatcctgcccacgcttgtgccctggtatttctggggtgaaacttttcaaaacagtgtgttcgttgccactttcttgcgatatgctgtggtgcttaatgccacctggctggtgaacagtgctgcccacctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaccacattcttcattgattgcatggccgccctcggtctggcctatgaccggaagaaagtctccaaggccgccatcttggccaggattaaaagaaccggagatggaaactacaagagtggctgagtttggggtccctcaggttcctttttcaaaaaccagccaggcagaggttttaatgtctgtttattaactactgaataatgctaccaggatgctaaagatgatgatgttaacccattccagtacagtattcttttaaaattcaaaagtattgaaagccaaaaaaaaaaaaaaaaaa

SEQ ID NO: 54 - DNA, 5221 bp, Homo sapiens
  ref <308>: DATABASE ACCESSION NUMBER: Genbank / AF097514
  ref <309>: DATABASE ENTRY DATE: 1999-05-19
  ref <313>: RELEVANT RESIDUES IN SEQ ID NO: (1)..(5221)
ataaaagggggctgaggaaataccggacacggtcacccgttgccagctctagcctttaaattcccggctcggggacctccacgcaccgcggctagcgccgacaaccagctagcgtgcaaggcgccgcggctcagcgcgtaccggcgggcttcgaaaccgcagtcctccggcgaccccgaactccgctccggagcctcagccccctggaaagtgatcccggcatccgagagccaagatgccggcccacttgctgcaggacgatatctctagctcctataccaccaccaccaccattacagcgcctccctccagggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgctgatgtgcttcatcctgcccacgcttgtgccctggtatttctggggtgaaacttttcaaaacagtgtgttcgttgccactttcttgcgatatgctgtggtgcttaatgccacctggctggtgaacagtgctgcccacctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaccacattcttcattgattgcatggccgccctcggtctggcctatgaccggaagaaagtctccaaggccgccatcttggccaggattaaaagaaccggagatggaaactacaagagtggctgagtttggggtccctcaggtttcctttttcaaaaaccagccaggcagaggttttaatgtctgtttattaactactgaataatgctaccaggatgctaaagatgatgatgttaacccattccagtacagtattcttttaaaattcaaaagtattgaaagccaacaactctgcctttatgatgctaagctgatattatttcttctcttatcctctctctcttctaggcccattgtcctccttttcactttattgctatcgccctcctttcccttattgcctcccaggcaagcagctggtcagtctttgctcagtgtccagcttccaaagcctagacaacctttctgtagcctaaaacgaatggtctttgctccagataactctctttccttgagctgttgtgagctttgaagtaggtggcttgagctagagataaaacagaatcttctgggtagtcccctgttgattatcttcagcccaggcttttgctagatggaatggaaaagcaacttcatttgacacaaagcttctaaagcaggtaaattgtcgggggagagagttagcatgtatgaatgtaaggatgagggaagcgaagcaagaggaacctctcgccatgatcagacatacagctgcctacctaatgaggacttcaagccccaccacatagcatgcttcctttctctcctggctcggggtaaaaagtggctgcggtgtttggcaatgctaattcaatgccgcaacatatagttgaggccgaggataaagaaaagacattttaagtttgtagtaaaagtggtctctgctggggaagggttttcttttctttttttctttaataacaaggagatttcttagttcatatatcaagaagtcttgaagttgggtgtttccagaattggtaaaaacagcagctcatggaattttgagtattccatgagctgctcattacagttctttcctctttctgctctgccatcttcaggatattggttcttcccctcatagtaataagatggctgtggcatttccaaacatccaaaaaaagggaaggatttaaggaggtgaagtcgggtcaaaaataaaatatatatacatatatacattgcttagaacgttaaactattagagtatttcccttccaaagagggatgtttggaaaaaactctgaaggagaggaggaattagttgggatgccaatttcctctccactgctggacatgagatggagaggctgagggacaggatctataggcagcttctaagagcgaacttcacataggaagggatctgagaacacgttgccaggggcttgagaaggttactgagtgagttattgggagtcttaataaaataaactagatattaggtccattcattaattagttccagtttctccttgaaatgagtaaaaactagaaggcttctctccacagtgttgtgccccttcactcatttttttttgaggagaagggggtctctgttaacatctagcctaaagtatacaactgcctggggggcagggttaggaatctcttcactaccctgattcttgattcctggctctaccctgtctgtcccttttctttgaccagatctttctcttccctgaacgttttcttctttccctggacaggcagcctcctttgtgtgtattcagaggcagtgatgacttgctgtccaggcagctccctcctgcacacagaatgctcagggtcactgaaccactgcttctcttttgaaagtagagctagctgccactttcacgtggcctccgcagtgtctccacctacacccctgtgctcccctgccacactgatggctcaagacaaggctggcaaaccctcccagaaacatctctggcccagaaagcctctctctccctccctctctcatgaggcacagccaagccaagcgctcatgttgagccagtgggccagccacagagcaaaagagggtttattttcagtcccctctctctgggtcagaaccagagggcatgctgaatgccccctgcttacttggtgagggtgccccgcctgagtcagtgctctcagctggcagtgcaatgcttgtagaagtaggaggaaacagttctcactgggaagaagcaagggcaagaacccaagtgcctcacctcgaaaggaggccctgttccctggagtcagggtgaactgcaaagctttggctgagacctgggatttgagataccacaaaccctgctgaacacagtgtctgttcagcaaactaaccagcattccctacagcctagggcagacaatagtatagaagtctggaaaaaaacaaaaacagaatttgagaaccttggaccactcctgtccctgtagctcagtcatcaaagcagaagtctggctttgctctattaagattggaaatgtacactaccaaacactcagtccactgttgagccccagtgctggaagggaggaaggcctttcttctgtgttaattgcgtagaggctacaggggttagcctggactaaaggcatccttgtcttttgagctattcacctcagtagaaaaggatctaagggaagatcactgtagtttagttctgttgacctgtgcacctaccccttggaaatgtctgctggtatttctaattccacaggtcatcagatgcctgcttgataatatataaacaataaaaacaactttcacttcttcctattgtaatcgtgtgccatggatctgatctgtaccatgaccctacataaggctggatggcacctcaggctgagggccccaatgtatgtgtggctgtgggtgtgggtgggagtgtgtctgctgagtaaggaacacgattttcaagattctaaagctcaattcaagtgacacattaatgataaactcagatctgatcaagagtccggatttctaacagtccctgctttggggggtgtgctgacaacttagctcaggtgccttacatcttttctaatcacagtgttgcatatgagcctgccctcactccctctgcagaatccctttgcacctgagaccctactgaagtggctggtagaaaaaggggcctgagtggaggattatcagtatcacgatttgcaggattcccttctgggcttcattctggaaacttttgttagggctgcttttcttaagtgcccacatttgatggagggtggaaataatttgaatgtatttgatttataagtttttttttttttttgggttaaaagatggttgtagcatttaaaatggaaaattttctccttggtttgctagtatcttgggtgtattctctgtaagtgtagctcaaataggtcatcatgaaaggttaaaaaagcgaggtggccatgttatgctggtggttaaggccagggcctctccaaccactgtgccactgacttgctgtgtgaccctgggcaagtcacttaactataaggtgcctcagttttccttctgttaaaatggggataataatactgacctacctcaaagggcagttttgaggcatgactaatgctttttagaaagcattttgggatccttcagcacaggaattctcaagacctgagtattttttataataggaatgtccaccatgaacttgatacgtccgtgtgtcccagatgctgtcattagtctatatggttctccaagaaactgaatgaatccattggagaagcggtggataactagccagacaaaatttgagaatacataaacaacgcattgccacggaaacatacagaggatgccttttctgtgattgggtgggattttttccctttttatgtgggatatagtagttacttgtgacaaaaataattttggaataatttctattaatatcaactctgaagctaattgtactaatctgagattgtgtttgttcataataaaagtgaagtgaatctaaaaaaaaaaaaaaa

SEQ ID NO: 55 - DNA, 5329 bp, Homo sapiens
  misc_feature (1986)..(1986): n is a, c, g, or t
  ref <308>: DATABASE ACCESSION NUMBER: Genbank / AB032261
  ref <309>: DATABASE ENTRY DATE: 2002-07-23
  ref <313>: RELEVANT RESIDUES IN SEQ ID NO: (1)..(5329)
gtggtgtcggtgtcggcagcatccccggcgccctgctgcggtcgccggagccctcggcctctgttctcctccccctcccgcccttacctccacgcgggaccgcccgcgccagtcaactcctcgcactttgcccctgcttggcagcggataaaagggggctgaggaaataccggacacgtccacccgttgccagctctagcctttaaattcccggctcgggacctccacgcaccgggctagcgccgacaaccagctagcgtgcaaggcgccgcggctcagcgcgtaccggcgggcttcgaaaccgcagtcctccggcgaccccgaactccgctccggagcctcagccccctggaaagtgatcccggcatcggagagccaagatgccggcccacttgctgcaggacgatatctctagctcctataccaccaccaccaccattacagcgcctccctccagggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgctgatgtgcttcatcctgcccacgcttgtgccctggtatttctggggtgaaacttttcaaaacagtgtgttcgttgccactttcttgcgatatgctgtggtgcttaatgccacctggctggtgaacagtgctgcccacctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaccacattcttcattgattgcatggccgccctcggtctggcctatgaccggaagaaagtctccaaggccgccatcttggccaggattaaaagaaccggagatggaaactacaagagtggctgagtttggggtccctcaggttcctttttcaaaaaccagccaggcagaggttttaatgtctgtttattaactactgaataatgctaccaggatgctaaagatgatgatgttaacccattccagtacagtattcttttaaaattcaaaagtattgaaagccaacaactctgcctttatgatgctaagctgatattatttcttctcttatcctctctctcttctaggcccattgtcctccttttcactttaatcgccctcctttcccttattgcctcccaggcaagcagctggtcagtctttgctcagtgtccagcttccaaagcctagacaacctttctgtagcctaaaacgaatggtctttgctccagataactctctttccttgagctgttgtgagctttgaagtaggtggcttgagctagagataaaacagaatcttctgggtagtcccctgttgattatcttcagcccaggcttttgctagatggaatggaaaagcaacttcatttgacacaaagcttctaaagcnaggtaaattgtcgggggagagagttagcatgtatgaatgtaaggatgagggaagcgaaggaacctctcgccatgatcagacatacagctgcctacctaatgaggacttcaagccccaccacatagcatgcttcctttctctcctggctcggggtaaaaagtggctgcggtgtttggcaatgctaattcaatgccgcaacatatagttgaggccgaggataaagaaaagacattttaagtttgtagtaaaagtggtctctgctggggaagggttttcttttctttttttctttaataacaaggagatttcttagttcatatatcaagaagtcttgaagttgggtgtttccagaattggtaaaaacagcagctcatagaattttgagtattccatgagctgctcattacagttctttcctctttctgctctgccatcttcaggatattggttcttcccctcatagtaataagatggctgtggcatttccaaacatccaaaaaaagggaaggatttaaggaggtgaagtcgggtcaaaaataaaatatatatacatatatacattgcttagaacgttaaactattagagtatttcccttccaaagagggatgtttggaaaaaactctgaaggagaggaggaattagttgggatgccaatttcctctccactgctggacatgagatggagaggctgagggacaggatctataggcagcttctaagagcgaacttcacataggaagggatctgagaacacgttcaggggttgagaaggttactgagtgagttattgggagtcttaataaactagatattaggtccattcattaattagttccagtttctccttgaaatgagtaaaaactagaaggcttctctccacagtgttgtgccccttcactcatttttttttgaggagaagggggtctctgttaacatctagcctaaagtatacaaactgcctggggggcagggttaggaatctcttcactaccctgattcttgattcctggctctaccctgtctgtcccttttctttgaccagatctttctcttccctgaacgttttcttctttccctggacaggcagcctcctttgtgtgtattcagaggcagtgatgacttgctgtccaggcagctccctcctgcacacagaatgctcagggtcactgaaccactgcttctcttttgaaagtagagctagctgccactttcacgtggcctccgcagtgtctccacctacacccctgtgctcccctgccacactgatggctcaagacaaggctggcaaaccctcccagaaacatctctggcccagaaagcctctctctccctccctctctcatgagaagccaagcgctcatgttgagccagtgggccagccacagagcaaaagagggtttattttcagtcccctctctctgggtcagaaccagagggcatgctgaatgccccctgcttacttggtgagggtgccccgcctgagtcagtgctctcagctggcagtgcaatgcttgtagaagtaggaggaaacagttctcactgggaagaagcaagggcaagaacccaagtgcctcacctcgaaaggaggccctgttccctggagtcagggtgaactgcaaagctttggctgagacctgggatttgagataccacaaaccctgctgaacacagtgtctgttcagcaaactaaccagcattccctacagcctagggcagacaatagtatagaagtctggaaaaaaacaaaaacagaatttgagaaccttggaccactcctgtccctgtagctcagtcatcaaagcagaagtctggctttgctctattaagattggaaatgtacactaccaaacactcagtccactgttgagccccagtgctggaagggaggaaggcctttcttctgtgttaattgcgtagaggctacaggggttagcctggactaaaggcatccttgtctttgagctattcacctcagtagaaaaggatctaagggaagatcactgtagtttagttctgttgacctgtgcacctaccccttggaaatgtctgctggtatttctaattccacaggtcatcagatgcctgcttgataatatataaacaataaaaacaactttcacttcttcctattgtaatcgtgtgccatggatctgatctgtaccatgaccctacataaggctggatggcacctcaggctgagggccccaatgtatgtgtggctgtgggtgtgggtgggagtgtgtctgctgagtaaggaacacgattttcaagattctaaagctcaattcaagtgacacattaatgataaactcagatctgatcaagagtccggatttctaacagtccttgctttggggggtgtgctggcaacttagctcaggtgccttacatcttttctaatcacagtgttgcatatgagcctgccctcactccctctgcagaatccctttgcacctgagaccctactgaagtggctggtagaaaaaggggcctgagtggaggattatcagtatcacgatttgcaggattcccttctgggcttcattctggaaacttttgttagggctgcttttcttaagtgcccacatttgatggagggtggaaataatttgaatgtatttgatttataagtttttttttttttttgggttaaaagatggttgtagcatttaaaatggaaaattttctccttggtttgctagtatcttgggtgtattctctgtaagtgtagctcaaataggtcatcatgaaaggttaaaaaagcgaggtggccatgttatgctggtggttgccagggcctccaaccactgtgccactgacttgctgtgtgaccctgggcaagtcacttaactataaggtgcctcagttttccttctgttaaaatggggataataatactgacctacctcaaagggcagttttgaggcatgactaatgctttttagaaagcattttgggatccttcagcacaggaattctcaagacctgagtattttttataataggaatgtccaccatgaacttgatacgtccgtgtgtcccagatgctgtcattagtctatatggttctccaagaaactgaatgaatccattggagaagcggtggataactagccagacaaaatttgagaatacataaacaacgcattgccacggaaacatacagaggatgccttttctgtgattgggtgggattttttccctttttatgtgggatatagtagttacttgtgacaagaataattttggaataatttctattaatatcaactctgaagctaattgtactaatctgagattgtgtttgttcataataaaagtgaagtgaatctgattgcactg

SEQ ID NO: 56 - DNA, 2150 bp, Homo sapiens
  ref <308>: DATABASE ACCESSION NUMBER: Genbank / NM_004508
  ref <309>: DATABASE ENTRY DATE: 2006-04-16
  ref <313>: RELEVANT RESIDUES IN SEQ ID NO: (1)..(2150)
ctcaggttcccggtgcttcgcgcgtctgccgttgtcacaggccagggaggtggcaccaccaggcgaagcttggcgagattgtgtcgtcaagcgcgtacgggcgccaattggccgggcgatgtggcgtggactggcgctggcgcgagcgattggctgcgcggcccgggggcggggccagtgggcggtgcgcgccgcagactgtgctcaaagcgggcgccatccgggaccggcggttgtctgtggccggaggctgatcagtgttctagaacagatcagacattttgtaatgatgcctgaaataaacactaaccacctcgacaagcaacaggttcaactcctggcagagatgtgtatccttattgatgaaaatgacaataaaattggagctgagaccaagaagaattgtcacctgaacgagaacattgagaaaggattattgcatcgagcttttagtgtcttcttattcaacaccgaaaataagcttctgctacagcaaagatcagatgctaagattacctttccaggttgttttacgaatacgtgttgtagtcatccattaagcaatccagccgagcttgaggaaagtgacgcccttggagtgaggcgagcagcacagagacggctgaaagctgagctaggaattcccttggaagaggttcctccagaagaaattaattatttaacacgaattcactacaaagctcagtctgatggtatctggggtgaacatgaaattgattacattttgttggtgaggaagaatgtaactttgaatccagatcccaatgagattaaaagctattgttatgtgtcaaaggaagaactaaaagaacttctgaaaaaagcagccagtggtgaaattaagataacgccatggtttaaaattattgcagcgacttttctctttaaatggtgggataacttaaatcatttgaatcagtttgttgaccatgagaaaatatacagaatgtgaatatgtaggtaaatgattacagaaaaatttatctgcttaacaaacttagaatgactttttccttttaaatttagttctatcattaatttatcattaaatttagttctatcatttggtactatcattaatgtattatatacactgatactttaaaacttgtgtggaaaaaactaacttataattttgtatcacacaccctggatatgtgttctgtttctaagcgacatttgtgagagattattgtaaaatgagagcgagcaaataaaacttaatttaatctttgcagatacatacttatgggaaatttgaacaaatgagtgaaactctgtttttagtaggctgtgataaacatttccggagcacttgcagaggacttgctatttgccaggtgctttatgtatcattaaatttttctcatagttcagaaaaatgtgcaaaggaaactattgtctcgctccttcaaaacagtcttaattaactttcatattagcagattaaactagcagagcaggttcaagggaaattaaatgatatggaccctaatttgtatcattctgagttgattgtgtggtttattcattctggaaacatgttgatacttacagtcagccactgcttttgataagtgatattgattaggttgaatcttcttgtaaatagtatttaccagttagcaaagtctgtgttttcagaattacagtgagcacagaggtgttcataaaatgggaattgagtcccactcggtaagagttgcttaaacttgacactgttgacatttgggctggataaaacccctgtggtggggtctgtgctgtgcattgcaggatggtgagcagcgtccctctcatgtgacacccacagttatgccggatgttgccagatgcccctaggggacagagtcaacccccaactgaggaccactgtcctacagagtcaggaaatattgtagggagaaaaaaataacaacaacaaaggcctgtgttaatgttaaatagatgagattatggaatgtgtatattaatgttaaaaattgtaccttgatcaatgtactttttataaacttgccatagatatctcagatttgaaacctcaagacagatttattattcttaaatgctgtatgataatgaagaaaaataaaaatttatttcttgcaaagttaaaaaaaaaaaaaaaa

SEQ ID NO: 57 - DNA, 2363 bp, Homo sapiens
  ref <308>: DATABASE ACCESSION NUMBER: Genbank / BC057827
  ref <309>: DATABASE ENTRY DATE: 2005-08-03
  ref <313>: RELEVANT RESIDUES IN SEQ ID NO: (1)..(2363)
ggcttccgccccgcagccccgaggggtctgggcgcgctgaagaccccttcggctgctgggccaacgcccccacccttggtcacctactcctgccgccgagcggttcgcgggaggtggcctcgggggtcgtagctactcaccggcggggcgctccgagtccatctgccccgtagcccggcagccggggcgggagggctgcgagcccggggcgcggtccgcctgaggcctgagacccggggccgcctgcgctcagggccgtggtgccggccgcgcgggagcctggcggggacggcgcggtcagtgttcggctcctccgcagtgactcaccagcgtcctgaagcttttgatcaccattggctgggaaggaaaaacagaatcagctgttccctgcgaaaggtatcatatgtttcttacaaagtggaggtttgtggcagccctgcatggagcaagtctgtcagcaccatctttctaacatgttctagaacagatcagacattttgtaatgatgcctgaaataaacactaaccacctcgacaagcaacaggttcaactcctggcagagatgtgtatccttattgatgaaaatgacaataaaattggagctgagaccaagaagaattgtcacctgaacgagaacattgagaaaggattattgcatcgagcttttagtgtcttcttattcaacaccgaaaataagcttctgctacagcaaagatcagatgctaagattacctttccaggttgttttacgaatacgtgttgtagtcatccattaagcaatccagccgagcttgaggaaagtgacgcccttggagtgaggcgagcagcacagagacggctgaaagctgagctaggaattcccttggaagaggttcctccagaagaaattaattatttaacacgaattcactacaaagctcagtctgatggtatctggggtgaacatgaaattgattacattttgttggtgaggaagaatgtaactttgaatccagatcccaatgagattaaaagctattgttatgtgtcaaaggaagaactaaaagaacttctgaaaaaagcagccagtggtgaaattaagataacgccatggtttaaaattattgcagcgacttttctctttaaatggtgggataacttaaatcatttgaatcagtttgttgaccatgagaaaatatacagaatgtgaatatgtaggtaaatgattacagaaaaatttatctgcttaacaaacttagaatgactttttccttttaaatttagttctatcattaatttatcattaaatttagttctatcatttggtactatcattaatgtattatatacactgatactttaaaacttgtgtggaaaaaactaacttataattttgtatcacacaccctggatatgtgttctgtttctaagcgacatttgtgagagattattgtaaaatgagagcgagcaaataaaacttaatttaatctttgcagatacatacttatgggaaatttgaacaaatgagtgaaactctgtgtttttagtaggctgtgataaacatttccggagcacttgcagaggacttgctatttgccaggtgctttatgtatcattaaatttttctcatagttcagaaaaatgtgcaaaggaaactattgtctcgctccttcaaaacagtcttaattaactttcatattagcagattaaactagcagagcaggttcaagggaaattaaatgatatggaccctaatttgtatcattctgagttgattgtgtggtttattcattctggaaacatgttgatacttacagtcagccactgcttttgataagtgatattgattaggttgaatcttcttgtaaatagtatttaccagttagcaaagtctgtgttttcagaattacagtgagcacagaggtgttcataaaatgggaattgagtcccactcggtaagagttgcttaaacttgacactgttgacatttgggctggataaaacccctgtggtggggtctgtgctgtgcattgcaggatggtgagcagcgtccctctcatgtgacacccacagttatgccggatgttgccagatgcccctaggggacagagtcaacccccaactgaggaccactgtcctacagagtcaggaaatattgtagggagaaaaaaataacaacaacaaaggcctgtgttaatgttaaatagatgagattatggaatgtgtatattaatgttaaaaattgtaccttgatcaatgtactttttataaacttgccatagatatctcagatttgaaacctcaagacagatttattattcttaaatgctgtatgataatgaagaaaaataaaaatttatttcttacaaaaaaaaaaaaaaaaa

SEQ ID NO: 58 - DNA, 1288 bp, Homo sapiens
  ref <308>: DATABASE ACCESSION NUMBER: Genbank / BC019227
  ref <309>: DATABASE ENTRY DATE: 2005-08-03
  ref <313>: RELEVANT RESIDUES IN SEQ ID NO: (1)..(1288)
gtgttctagaacagatcagacattttgtaatgatgcctgaaataaacactaaccacctcgacaagcaacaggttcaactcctggcagagatgtgtatccttattgatgaaaatgacaataaaattggagctgagaccaagaagaattgtcacctgaacgagaacattgagaaaggattattgcatcgagcttttagtgtcttcttattcaacaccgaaaataagcttctgctacagcaaagatcagatgctaagattacctttccaggttgttttacgaatacgtgttgtagtcatccattaagcaatccagccgagcttgaggaaagtgacacccttggagtgaggcgagcagcacagagacggctgaaagctgagctaggaattcccttggaagaggttcctccagaagaaattaattatttaacacgaattcactacaaagctcagtctgatggtatctggggtgaacatgaaattgattacattttgttggtgaggaagaatgtaactttgaatccagatcccaatgagattaaaagctattgttatgtgtcaaaggaagaactaaaagaacttctgaaaaaagcagccagtggtgaaattaagataacgccatggtttaaaattattgcagcgacttttctctttaaatggtgggataacttaaatcatttgaatcagtttgttgaccatgagaaaatatacagaatgtgaatatgtaggtaaatgattacagaaaaatttatctgcttaacaaacttagaatgactttttccttttaaatttagttctatcattaatttatcattaaatttagttctatcatttggtgctatcattaatgtattatatacactgatactttaaaacttgtgtggaaaaaactaacttataattttgtatcacacaccctggatatgtgttctgtttctaagcgacatttgtgagagattattgtaaaatgagagcgagcaaataaaacttaatttaatctttgcagatacatacttatgggaaatttgaacaaatgagtgaaactctgtgtttttagtaggctgtgataaacatttccggagcacttgcagaggacttgctatttgccaggtgctttatgtatcattaaatttttctcatagttcagaaaaatgtgcaaaggaaactattgtctcgctccttcaaaacagtcttaattaactttcatattagcagattaaactagcagagcaggttcaagggaaattaaatgatatggacccgaaaaaaaaaaaaaaaaaaaaaaaaaaaaa

SEQ ID NO: 59 - DNA, 1903 bp, Homo sapiens
  ref <308>: DATABASE ACCESSION NUMBER: Genbank / BC022418
  ref <309>: DATABASE ENTRY DATE: 2005-08-03
  ref <313>: RELEVANT RESIDUES IN SEQ ID NO: (1)..(1903)
gtgttctagaacagatcagacattttgtaatgatgcctgaaataaacactaaccacctcgacaagcaacaggttcaactcctggcagagatgtgtatccttattgatgaaaatgacaataaaattggagctgagaccaagaagaattgtcacctgaacgagaacattgagaaaggattattgcatcgagcttttagtgtcttcttattcaacaccgaaaataagcttctgctacagcaaagatcagatgctaagattacctttccaggttgttttacgaatacgtgttgtagtcatccattaagcaatccagccgagcttgaggaaagtgacgcccttggagtgaggcgagcagcacagagacggctgaaagctgagctaggaattcccttggaagaggttcctccagaagaaattaattatttaacacgaattcactacaaagctcagtctgatggtatctggggtgaacatgaaattgattacattttgttggtgaggaagaatgtaactttgaatccagatcccaatgagattaaaagctattgttatgtgtcaaaggaagaactaaaagaacttctgaaaaaagcagccagtggtgaaattaagataacgccatggtttaaaattattgcagcgacttttctctttaaatggtgggataacttaaatcatttgaatcagtttgttgaccatgagaaaatatacagaatgtgaatatgtaggtaaatgattacagaaaaatttatctgcttaacaaacttagaatgactttttccttttaaatttagttctatcattaatttatcattaaatttagttctatcatttggtactatcattaatgtattatatacactgatactttaaaacttgtgtggaaaaaactaacttataattttgtatcacacaccctggatatgtgttctgtttctaagcgacatttgtgagagattattgtaaaatgagagcgagcaaataaaacttaatttaatctttgcagatacatacttatgggaaatttgaacaaatgagtgaaactctgtgtttttagtaggctgtgataaacatttccggagcacttgcagaggacttgctatttgccaggtgctttatgtatcattaaatttttctcatagttcagaaaaatgtgcaaaggaaactattgtctcgctccttcaaaacagtcttaattaactttcatattagcagattaaactagcagagcaggttcaagggaaattaaatgatatggaccctaatttgtatcattctgagttgattgtgtggtttattcattctggaaacatgttgatacttacagtcagccactgcttttgataagtgatattgattaggttgaatcttcttgtaaatagtatttaccagttagcaaagtctgtgttttcagaattacagtgagcacagaggtgttcataaaatgggaattgagtcccactcggtaagagttgcttaaacttgacactgttgacatttgggctggataaaacccctgtggtggggtctgtgctgtgcattgcaggatggtgagcagcgtccctctcatgtgacacccacagttatgccggatgttgccagatgcccctaggggacagagtcaacccccaactgaggaccactgtcctacagagtcaggaaatattgtagggagaaaaaaataacaacaacaaaggcctgtgttaatgttaaatagatgagattatggaatgtgtatattaatgttaaaaattgtaccttgatcaatgtactttttataaacttgccatagatatctcagatttgaaacctcaagacagatttattattcttaaatgctgtatgataatgaagaaaaataaaaatttatttcttgcgaaaaaaaaaaaaaaaaaaaaaaaaaaaaa

SEQ ID NO: 60 - DNA, 1902 bp, Homo sapiens
  ref <308>: DATABASE ACCESSION NUMBER: Genbank / BC025375
  ref <309>: DATABASE ENTRY DATE: 2005-08-03
  ref <313>: RELEVANT RESIDUES IN SEQ ID NO: (1)..(1902)
gtgttctagaacagatcagacattttgtaatgatgcctgaaataaacactaaccacctcgacaagcaacaggttcaactcctggcagagatgtgtatccttattgatgaaaatgacaataaaattggagctgagaccaagaagaattgtcacctgaacgagaacattgagaaaggattattgcatcgagcttttagtgtcttcttattcaacaccgaaaataagcttctgctacagcaaagatcagatgctaagattacctttccaggttgttttacgaatacgtgttgtagtcatccattaagcaatccagccgagcttgaggaaagtgacgcccttggagtgaggcgagcagcacagagacggctgaaagctgagctaggaattcccttggaagaggttcctccagaagaaattaattatttaacacgaattcactacaaagctcagtctgatggtatctggggtgaacatgaaattgattacattttgttggtgaggaagaatgtaactttgaatccagatcccaatgagattaaaagctattgttatgtgtcaaaggaagaactaaaagaacttctgaaaaaagcagccagtggtgaaattaagataacgccatggtttaaaattattgcagcgacttttctctttaaatggtgggataacttaaatcatttgaatcagtttgttgaccatgagaaaatatacagaatgtgaatatgtaggtaaatgattacagaaaaatttatctgcttaacaaacttagaatgactttttccttttaaatttagttctatcattaatttatcattaaatttagttctatcatttggtactatcattaatgtattatatacactgatactttaaaacttgtgtggaaagactaacttataattttgtatcacacaccttggatatgtgttctgtttctaagcgacatttgtgagagattattgtaaaatgagagagagcaaataaaacttaatttaatctttgcagatacatacttatgggaaatttgaacaaatgagtgaaactctgtgtttttagtaggctgtgataaacatttccggagcacttgcagaggacttgctatttgccaggtgctttatgtatcattaaatttttctcatagttcagaaaaatgtgcaaaggaaactattgtctcgctccttcaaaacagtcttaattaactttcatattagcagattaaactagcagagcaggttcaagggaaattaaatgatatggaccctaatttgtatcattctgagttgattgtgtggtttattcattctggaaacatgttgatacttacagtcagccactgcttttgataagtgatattgattaggttgaatcttcttgtaaatagtatttaccagttagcaaagtctgtgttttcagaattacagtgagcacagaggtgttcataaaatgggaattgagtcccactcggtaagagttgcttaaacttgacactgttgacatttgggctggataaaacccctgtggtggggtctgtgctgtgcattgcaggatggtgagcagcgtccctctcatgtgacacccacagttatgccggatgttgccagatgcccctaggggacagagtcaacccccaactgaggaccactgtcctacagagtcaggaaatattgtagggagaaaaaaataacaacaacaaaggcctgtgttaatgttaaatagatgagattatggaatgtgtatattaatgttaaaaattgtaccttgatcaatgtactttttataaacttgccatagatatctcagatttgaaacctcaagacagatttattattcttaaatgctgtatgataatgaagaaaaataaaaatttatttcttgcaaaaaaaaaaaaaaaaaaaaaaaaaaaaaa

SEQ ID NO: 61 - DNA, 2024 bp, Homo sapiens
  ref <308>: DATABASE ACCESSION NUMBER: Genbank / BC006999
  ref <309>: DATABASE ENTRY DATE: 2005-08-03
  ref <313>: RELEVANT RESIDUES IN SEQ ID NO: (1)..(2024)
gtgttctagaacagatcagacattttgtaatgatgcctgaaataaacactaaccacctcgacaagcaacaggttcaactcctggcagagatgtgtatccttattgatgaaaatgacaataaaattggagctgagaccaagaagaattgtcacctgaacgagaacattgagaaaggattattgcatcgagcttttagtgtcttcttattcaacaccgaaaataagcttctgctacagcaaagatcagatgctaagattacctttccaggttgttttacgaatacgtgttgtagtcatccattaagcaatccagccgagcttgaggaaagtgacgcccttggagtgaggcgagcagcacagagacggctgaaagctgagctaggaattcccttggaagaggttcctccagaagaaattaattatttaacacgaattcactacaaagctcagtctgatggtatctggggtgaacatgaaattgattacattttgttggtgaggaagaatgtaactttgaatccagatcccaatgagattaaaagctattgtcatgtgtcaaaggaagaactaaaagaacttctgaaaaaagcagccagtggtgaaattaagataacgccatggtttaaaattattgcagcgacttttctctttaaatggtgggataacttaaatcatttgaatcagtttgttgaccatgagaaaatatacagaatgtgaatatgtaggtaaatgattacagaaaaatttatctgcttaacaaacttagaatgactttttccttttaaatttagttctatcattaatttatcattaaatttagttctatcatttggtactatcattaatgtattatatacactgatactttaaaacttgtgtggaaaaaactaacttataattttgtatcacacaccctggatatgtgttctgtttctaagcgacatttgtgagagattattgtaaaatgagagcgagcaaataaaacttaatttaatctttgcagatacatacttatgggaaatttgaacaaatgagtgaaactctgtgtttttagtaggctgtgataaacatttccggagcacttgcagaggacttgctatttgccaggtgctttatgtatcattaaatttttctcatagttcagaaaaatgtgcaaaggaaactattgtctcgctccttcaaaacagtcttaattaactttcatattagcagattaaactagcagagcaggttcaagggaaattaaatgatatggaccctaatttgtatcattctgagttgattgtgtggtttattcattctggaaacatgttgatacttacagtcagccactgcttttgataagtgatattgattaggttgaatcttcttgtaaatagtatttaccagttagcaaagtctgtgttttcagaattacagtgagcacagaggtgttcataaaatgggaattgagtcccactcggtaagagttgcttaaacttgacactgttgacatttgggctggataaaacccctgtggtggggtctgtgctgtgcattgcaggatggtgagcagcgtccctctcatgtgacacccacagttatgccggatgttgccagatgcccctaggggacagagtcaacccccaactgaggaccactgtcctacagagtcaggaaatattgtagggagaaaaaaataacaacaacaaaggcctgtgttaatgttaaatagatgagattatggaatgtgtatattaatgttaaaaattgtaccttgatcaatgtactttttataaacttgccatagatatctcagatttgaaacctcaagacagatttattattcttaaatgctgtatgataatgaagaaaaataaaaatttatttcttgcaaagttaaatgtttgttaaattcaatagaatgactcatttatggttaactttgggcaatttataatttcagacaagactgtttagcaagtattttattgaaaagtaaaaaaaattgcaatttaaaaaaaaaaaaaaaaaaaaaaaaaaaaa

SEQ ID NO: 62 - DNA, 1911 bp, Homo sapiens
  ref <308>: DATABASE ACCESSION NUMBER: Genbank / BC005247
  ref <309>: DATABASE ENTRY DATE: 2005-08-03
  ref <313>: RELEVANT RESIDUES IN SEQ ID NO: (1)..(1911)
gtgttctagaacagatcagacattttgtaatgatgcctgaaataaacactaaccacctcgacaagcaacaggttcaactcctggcagagatgtgtatccttattgatgaaaatgacaataaaattggagctgagaccaagaagaattgtcacctgaacgagaacattgagaaaggattattgcatcgagcttttagtgtcttcttattcaacaccgaaaataagcttctgctacagcaaagatcagatgctaagattacctttccaggttgttttacgaatacgtgttgtagtcatccattaagcaatccagccgagcttgaggaaagtgacgcccttggagtgaggcgagcagcacagagacggctgaaagctgagctaggaattcccttggaagaggttcctccagaagaaattaattatttaacacgaattcactacaaagctcagtctgatggtatctggggtgaacatgaaattgattacattttgttggtgaggaagaatgtaactttgaatccagatcccaatgagattaaaagctattgttatgtgtcaaaggaagaactaaaagaacttctgaaaaaagcagccagtggtgaaattaagataacgccatggtttaaaattattgcagcgacttttctctttaaatggtgggataacttaaatcatttgaatcagtttgttgaccatgagaaaatatacagaatgtgaatatgtaggtaaatgattacagaaaaatttatctgcttaacaaacttagaatgactttttccttttaaatttagttctatcattaatttatcattaaatttagttctatcatttggtactatcattaatgtattatatacactgatactttaaaacttgtgtggaaaaaactaacttataattttgtatcacacaccctggatatgtgttctgtttctaagcgacatttgtgagagattattgtaaaatgagagcgagcaaataaaacttaatttaatctttgcagatacatacttatgggaaatttgaacaaatgagtgaaactctgtgtttttagtaggctgtgataaacatttccggagcacttgcagaggacttgctatttgccaggtgctttatgtatcattaaatttttctcatagttcagaaaaatgtgcaaaggaaactattgtctcgctccttcaaaacagtcttaattaactttcatattagcagattaaactagcagagcaggttcaagggaaattaaatgatatggaccctaatttgtatcattctgagttgattgtgtggtttattcattctggaaacatgttgatacttacagtcagccactgcttttgataagtgatattgattaggttgaatcttcttgtaaatagtatttaccagttagcaaagtctgtgttttcagaattacagtgagcacagaggtgttcataaaatgggaattgagtcccactcggtaagagttgcttaaacttgacactgttgacatttgggctggataaaacccctgtggtggggtctgtgctgtgcattgcaggatggtgagcagcgtccctctcatgtgacacccacagttatgccggatgttgccagatgcccctaggggacagagtcaacccccaactgaggaccactgtcctacagagtcaggaaatattgtagggagaaaaaaataacaacaacaaaggcctgtgttaatgttaaatagatgagattatggaatgtgtatattaatgttaaaaattgtaccttgatcaatgtactttttataaacttgccatagatatctcagatttgaaacctcaagacagatttattattcttaaatgctgtatgataatgaagaaaaataaaaatttatttcttgcaaagttacaaaaaaaaaaaaaaaaaaaaaaaaaaaaaa

SEQ ID NO: 63 - DNA, 1773 bp, Homo sapiens
  ref <308>: DATABASE ACCESSION NUMBER: Genbank / AF271720
  ref <309>: DATABASE ENTRY DATE: 2001-05-02
  ref <313>: RELEVANT RESIDUES IN SEQ ID NO: (1)..(1773)
gatcagacattttgtaatgatgcctgaaataaacactaaccacctcgacaagcaacaggttcaactcctggcagagatgtgtatccttattgatgaaaatgacaataaaattggagctgagaccaagaagaattgtcacctgaacgagaacattgagaaaggattattgcatcgagcttttagtgtcttcttattcaacaccgaaaataagcttctgctacagcaaagatcagatgctaagattacctttccaggttgttttacgaatacgtgttgtagtcatccattaagcaatccagccgagcttgaggaaagtgacgcccttggagtgaggcgagcagcacagagacggctgaaagctgagctaggaattcccttggaagaggttcctccagaagaaattaattatttaacacgaattcactacaaagctcagtctgatggtatctggggtgaacatgaaattgattacattttgttggtgaggaagaatgtaactttgaatccagatcccaatgagattaaaagctattgttatgtgtcaaaggaagaactaaaagaacttctgaaaaaagcagccagtggtgaaattaagataacgccatggtttaaaattattgcagcgacttttctctttaaatggtgggataacttaaatcatttgaatcagtttgttgaccatgagaaaatatacagaatgtgaatatgtaggtaaatgattacagaaaaatttatgtgcttaacaaacttagaatgactttttccttttaaatttagttctatcattaatttatcattaaatttagttctatcatttggtactatcattaatgtattataaaacttgtgtggaaaaaactaacttataattttgtatcacacaccctggatatgtgttctgtttctaagcgacatttgtgagagattattgtaaaatgagagcgagaaataaaacttaatttaatctttgcagatacatacttatgggaaatttgaacaaatgagtgaaactctgtttttagtaggccgtgataaacatttccggagcacttgcagaggacttgctatttgccaggtgctttatgtatcattaaatttttctcatagttcagaaaaatgtgcaaaggaaactattgtctcgctccttcaaaacagtcttaattaactttcatattagcagattaaactagcagagcaaggttcaaattaaatgatatgaccctaatttgtatcattctgagttgattgtgtggtttattcattctgaaacatgttgatacttacagtcaccgactgcttttgataagtgatattgattaggttgaatcttcttgtaaatagtatttaccagttagcaaagtctgtgttttcagaattacagtgagcacagaggtgttcataaaatgggaattgagtcccactcggtaagagttgcttaaacttgacactgttgacatttgggctggataaaacccctgtggtggggtctgtgctgtgcattgcaggatggtgagcagcgtccctctcatgtgacacccacagttatgccggatgttgccagatgcccctagggacagagtcaacccccaactgaggaccactgtctacagagtcaggaaatattgtagggagaaaaaaataacaacaacaaaggcctatattaatgttaaatagaggagattatggaatgtgtatattaatgttaaaaattattccttattcaatgtatttttatcaaatcgatagatatctcagatttgaaactcaagacag

SEQ ID NO: 64 - protein, 69 aa, Homo sapiens
  ref <308>: DATABASE ACCESSION NUMBER: Genbank / NP_055388
  ref <309>: DATABASE ENTRY DATE: 2006-02-16
  ref <313>: RELEVANT RESIDUES IN SEQ ID NO: (1)..(69)
MTTLIPILSTFLFEDFSKASGFKGQRPETLHERLTLVSVYAPYLLIPFILLIFMLRSPYYKYEEKRKKK

SEQ ID NO: 65 - protein, 176 aa, Homo sapiens
  ref <308>: DATABASE ACCESSION NUMBER: Genbank / AAH91504
  ref <309>: DATABASE ENTRY DATE: 2005-03-30
  ref <313>: RELEVANT RESIDUES IN SEQ ID NO: (1)..(176)
MGAPATRRCVEWLLGLYFLSHIPITLFMDLQAVLPRELYPVEFRNLLKWYAKEFKDPLLQEPPAWFKSFLFCELVFQLPFFPIATYAFLKGSCKWIRTPAIIYSVHTMTTLIPILSTFLFEDFSKASGFKGQRPETLHERLTLVSVYAPYLLIPFILLIFMLRSPYYKYEEKRKKK

SEQ ID NO: 66 - protein, 69 aa, Homo sapiens; identical to SEQ ID NO: 64
  ref <308>: DATABASE ACCESSION NUMBER: Genbank n/ AAH45655
  ref <309>: DATABASE ENTRY DATE: 2006-04-14
  ref <313>: RELEVANT RESIDUES IN SEQ ID NO: (1)..(69)

SEQ ID NO: 67 - protein, 189 aa, Homo sapiens
  ref <308>: DATABASE ACCESSION NUMBER: Genbank / AAA16188
  ref <309>: DATABASE ENTRY DATE: 1994-02-02
  ref <313>: RELEVANT RESIDUES IN SEQ ID NO: (1)..(189)
LFSHQRVQAQPTDYGGSFTRRCVEWLLGLYFLSHIPITLFMDLQAVVPRELYPVEFRNLLKWYAKEFKDPLLQEPPAWFKSFLFCELVFQLPFFPIATYAFLKGSCKWIRTPAIIYSVHTMTTLILILSTFLFEDFSKASGFKGQRPETLHERLTLVSVYAPYLLIPFILLIFMLRSPYYKYEEKRKKK

SEQ ID NO: 68 - protein, 125 aa, Homo sapiens
  ref <308>: DATABASE ACCESSION NUMBER: Genbank / AAH18737
  ref <309>: DATABASE ENTRY DATE: 2005-03-08
  ref <313>: RELEVANT RESIDUES IN SEQ ID NO: (1)..(125)
MDHKPLLQERPPAYNLEAGQGDYACGPHGYGAIPAAPPPPPYPYLVTGIPTHYPRVYNIHSRTVTRYPANSIVVVGGCPVCRVGVLEDCFTFLGIFLAIILFPFGFICCFALRKRRCPNCGATFA

SEQ ID NO: 69 - protein, 125 aa, Homo sapiens
  ref <308>: DATABASE ACCESSION NUMBER: Genbank / AAH71992
  ref <309>: DATABASE ENTRY DATE: 2004-06-08
  ref <313>: RELEVANT RESIDUES IN SEQ ID NO: (1)..(125)
MDHKPLLQERPPAYNLEAGQGDYACGPHGYGAIPAAPPPPPYPYLVTGIPTHHPRVYNIHSRTVTRYPANSIVVVGGCPVCRVGVLEDCFTFLGIFLAIILFPFGFICCFALRKRRCPNCGATFA

SEQ ID NO: 70 - protein, 125 aa, Homo sapiens; identical to SEQ ID NO: 69
  ref <308>: DATABASE ACCESSION NUMBER: Genbank / AAD05167
  ref <309>: DATABASE ENTRY DATE: 1999-01-15
  ref <313>: RELEVANT RESIDUES IN SEQ ID NO: (1)..(125)

SEQ ID NO: 71 - protein, 125 aa, Homo sapiens; identical to SEQ ID NO: 69
  ref <308>: DATABASE ACCESSION NUMBER: Genbank / BAB32785
  ref <309>: DATABASE ENTRY DATE: 2002-07-23
  ref <313>: RELEVANT RESIDUES IN SEQ ID NO: (1)..(125)

SEQ ID NO: 72 - protein, 125 aa, Homo sapiens; identical to SEQ ID NO: 69
  ref <308>: DATABASE ACCESSION NUMBER: Genbank / NP_056194
  ref <309>: DATABASE ENTRY DATE: 2005-09-24
  ref <313>: RELEVANT RESIDUES IN SEQ ID NO: (1)..(125)

SEQ ID NO: 73 - protein, 125 aa, Homo sapiens; identical to SEQ ID NO: 69
  ref <308>: DATABASE ACCESSION NUMBER: Genbank / AAH62370
  ref <309>: DATABASE ENTRY DATE: 2005-03-08
  ref <313>: RELEVANT RESIDUES IN SEQ ID NO: (1)..(125)

SEQ ID NO: 74 - protein, 125 aa, Homo sapiens; identical to SEQ ID NO: 69
  ref <308>: DATABASE ACCESSION NUMBER: Genbank / AAF18565
  ref <309>: DATABASE ENTRY DATE: 2002-04-17
  ref <313>: RELEVANT RESIDUES IN SEQ ID NO: (1)..(125)

SEQ ID NO: 75 - protein, 125 aa, Homo sapiens; identical to SEQ ID NO: 69
  ref <308>: DATABASE ACCESSION NUMBER: Genbank / O95415
  ref <309>: DATABASE ENTRY DATE: 2006-04-11
  ref <313>: RELEVANT RESIDUES IN SEQ ID NO: (1)..(125)

SEQ ID NO: 76 - protein, 134 aa, Homo sapiens
  ref <308>: DATABASE ACCESSION NUMBER: Genbank / NP_075010
  ref <309>: DATABASE ENTRY DATE: 2005-10-17
  ref <313>: RELEVANT RESIDUES IN SEQ ID NO: (1)..(134)
MRQKAVSLFLCYLLLFTCSGVEAGENAGKKKCSESSDSGSGFWKALTFMAVGGGLAVAGLPALGFTGAGIAANSVAASLMSWSAILNGGGVPAGGLVATLQSLGAGGSSVVIGNIGALMGYATHKYLDSEEDEE

SEQ ID NO: 77 - protein, 138 aa, Homo sapiens
  ref <308>: DATABASE ACCESSION NUMBER: Genbank / NP_075011
  ref <309>: DATABASE ENTRY DATE: 2005-10-17
  ref <313>: RELEVANT RESIDUES IN SEQ ID NO: (1)..(138)
MRQKAVSLFLCYLLLFTCSGVEAGENAGKDAGKKKCSESSDSGSGFWKALTFMAVGGGLAVAGLPALGFTGAGIAANSVAASLMSWSAILNGGGVPAGGLVATLQSLGAGGSSVVIGNIGALMGYATHKYLDSEEDEE

SEQ ID NO: 78 - protein, 130 aa, Homo sapiens
  ref <308>: DATABASE ACCESSION NUMBER: Genbank / NP_002029
  ref <309>: DATABASE ENTRY DATE: 2005-10-17
  ref <313>: RELEVANT RESIDUES IN SEQ ID NO: (1)..(130)
MRQKAVSLFLCYLLLFTCSGVEAGKKKCSESSDSGSGFWKALTFMAVGGGLAVAGLPALGFTGAGIAANSVAASLMSWSAILNGGGVPAGGLVATLQSLGAGGSSVVIGNIGALMGYATHKYLDSEEDEE

SEQ ID NO: 79 - protein, 130 aa, Homo sapiens; identical to SEQ ID NO: 78
  ref <308>: DATABASE ACCESSION NUMBER: Genbank / AAH15603
  ref <309>: DATABASE ENTRY DATE: 2004-06-29
  ref <313>: RELEVANT RESIDUES IN SEQ ID NO: (1)..(130)

SEQ ID NO: 80 - protein, 130 aa, Homo sapiens; identical to SEQ ID NO: 78
  ref <308>: DATABASE ACCESSION NUMBER: Genbank / CAE12275
  ref <309>: DATABASE ENTRY DATE: 2006-04-04
  ref <313>: RELEVANT RESIDUES IN SEQ ID NO: (1)..(130)

SEQ ID NO: 81 - protein, 130 aa, Homo sapiens; identical to SEQ ID NO: 78
  ref <308>: DATABASE ACCESSION NUMBER: Genbank / AAH11601
  ref <309>: DATABASE ENTRY DATE: 2005-03-08
  ref <313>: RELEVANT RESIDUES IN SEQ ID NO: (1)..(130)

SEQ ID NO: 82 - protein, 130 aa, Homo sapiens; identical to SEQ ID NO: 78
  ref <308>: DATABASE ACCESSION NUMBER: Genbank / AAP35496
  ref <309>: DATABASE ENTRY DATE: 2003-05-13
  ref <313>: RELEVANT RESIDUES IN SEQ ID NO: (1)..(130)

SEQ ID NO: 83 - protein, 130 aa, Homo sapiens [sequence omitted]

SEQ ID NO: 84 - protein, 129 aa, Homo sapiens
  ref <308>: DATABASE ACCESSION NUMBER: Genbank / AAH29131
  ref <309>: DATABASE ENTRY DATE: 2004-06-30
  ref <313>: RELEVANT RESIDUES IN SEQ ID NO: (1)..(129)
MADSGDAGSSGPWWKSLTNSRKKSKEAAVGVPPPAQPAPGEPTPPAPPSPDWTSSSRENQHPNLLGGAGEPPKPDKLYGDKSGSSRRNLKISRSGRFKEKRKVRATLLPEAGRSPEEAGFPGDPHEDKQ

SEQ ID NO: 85 - protein, 129 aa, Homo sapiens; identical to SEQ ID NO: 84
  ref <308>: DATABASE ACCESSION NUMBER: Genbank / NP_787083
  ref <309>: DATABASE ENTRY DATE: 2005-03-02
  ref <313>: RELEVANT RESIDUES IN SEQ ID NO: (1)..(129)

SEQ ID NO: 86 - protein, 129 aa, Homo sapiens; identical to SEQ ID NO: 84
  ref <308>: DATABASE ACCESSION NUMBER: Genbank / EAL24204
  ref <309>: DATABASE ENTRY DATE: 2004-08-10
  ref <313>: RELEVANT RESIDUES IN SEQ ID NO: (1)..(129)

SEQ ID NO: 87 - protein, 386 aa, Homo sapiens
  ref <308>: DATABASE ACCESSION NUMBER: Genbank / NP_060419
  ref <309>: DATABASE ENTRY DATE: 2005-09-23
  ref <313>: RELEVANT RESIDUES IN SEQ ID NO: (1)..(386)
MTSLFRRSSSGSGGGGTAGARGGGGGTAAPQELNNSRPARQVRRLEFNQAMDDFKTMFPNMDYDIIECVLRANSGAVDATIDQLLQMNLEGGGSSGGVYEDSSDSEDSIPPEILERTLEPDSSDEEPPPVYSPPAYHMHVFDRPYPLAPPTPPPRIDALGSGAPTSQRRYRNWNPPLLGNLPDDFLRILPQQLDSIQGNAGGPKPGSGEGCPPAMAGPGPGDQESRWKQYLEDERIALFLQNEEFMKELQRNRDFLLALERDRLKYESQKSKSSSVAVGNDFGFSSPVPGTGDANPAVSEDALFRDKLKHMGKSTRRKLFELARAFSEKTKMRKSKRKHLLKHQSLGAAASTANLLDDVEGHACDEDFRGRRQEAPKVEEGLREGQ

SEQ ID NO: 88 - protein, 386 aa, Homo sapiens; identical to SEQ ID NO: 87
  ref <308>: DATABASE ACCESSION NUMBER: Genbank / BAA91357
  ref <309>: DATABASE ENTRY DATE: 2003-09-15
  ref <313>: RELEVANT RESIDUES IN SEQ ID NO: (1)..(386)

SEQ ID NO: 89 - protein, 386 aa, Homo sapiens; identical to SEQ ID NO: 87
  ref <308>: DATABASE ACCESSION NUMBER: Genbank / AAH56882
  ref <309>: DATABASE ENTRY DATE: 2004-03-25
  ref <313>: RELEVANT RESIDUES IN SEQ ID NO: (1)..(386)

SEQ ID NO: 90 - protein, 358 aa, Homo sapiens [sequence omitted]

SEQ ID NO: 91 - protein, 386 aa, Homo sapiens; identical to SEQ ID NO: 87
  ref <308>: DATABASE ACCESSION NUMBER: Genbank / Q9NWM3
  ref <309>: DATABASE ENTRY DATE: 2006-04-14
  ref <313>: RELEVANT RESIDUES IN SEQ ID NO: (1)..(386)

SEQ ID NO: 92 - protein, 151 aa, Homo sapiens
  ref <308>: DATABASE ACCESSION NUMBER: Genbank / NP_006423
  ref <309>: DATABASE ENTRY DATE: 2006-04-16
  ref <313>: RELEVANT RESIDUES IN SEQ ID NO: (1)..(151)
MRFLAATFLLLALSTAAQAEPVQFKDCGSVDGVIKEVNVSPCPTQPCQLSKGQSYSVNVTFTSNIQSKSSKAVVHGILMGVPVPFPIPEPDGCKSGINCPIQKDKTYSYLNKLPVKSEYPSIKLVVEWQLQDDKNQSLFCWEIPVQIVSHL

SEQ ID NO: 93 - protein, 151 aa, Homo sapiens; identical to SEQ ID NO: 92
  ref <308>: DATABASE ACCESSION NUMBER: Genbank / AAH02532
  ref <309>: DATABASE ENTRY DATE: 2005-03-08
  ref <313>: RELEVANT RESIDUES IN SEQ ID NO: (1)..(151)

SEQ ID NO: 94 - protein, 151 aa, Homo sapiens
  ref <308>: DATABASE ACCESSION NUMBER: Genbank / BAD96194
  ref <309>: DATABASE ENTRY DATE: 2005-04-25
  ref <313>: RELEVANT RESIDUES IN SEQ ID NO: (1)..(151)
MRFLAATFLLLALSTAAQAEPVQFRDCGSVDGVIKEVNVSPCPTQPCQLSKGQSYSVNVTFTSNVQSKSSKAVVHGILMGVPVPFPIPEPDGCKSGINCPIQKDKTYSYLNKLPVKSEYPSIKLVVEWQLQDDKNQSLFCWEIPVQIVSHL

SEQ ID NO: 95 - protein, 151 aa, Homo sapiens; identical to SEQ ID NO: 92
  ref <308>: DATABASE ACCESSION NUMBER: Genbank / CAA47928
  ref <309>: DATABASE ENTRY DATE: 2003-08-05
  ref <313>: RELEVANT RESIDUES IN SEQ ID NO: (1)..(151)

SEQ ID NO: 96 - protein, 151 aa, Homo sapiens; identical to SEQ ID NO: 92
  ref <308>: DATABASE ACCESSION NUMBER: Genbank / P61916
  ref <309>: DATABASE ENTRY DATE: 2006-04-14
  ref <313>: RELEVANT RESIDUES IN SEQ ID NO: (1)..(151)

SEQ ID NO: 97 - protein, 359 aa, Homo sapiens
  ref <308>: DATABASE ACCESSION NUMBER: Genbank / BAA93510
  ref <309>: DATABASE ENTRY DATE: 2002-07-23
  ref <313>: RELEVANT RESIDUES IN SEQ ID NO: (1)..(359)
MPAHLLQDDISSSYTTTTTITAPPSRVLQNGGDKLETMPLYLEDDIRPDIKDDIYDPTYKDKEGPSPKVEYVWRNIILMSLLHLGALYGITLIPTCKFYTWLWGVFYYFVSALGITAGAHRLWSHRSYKARLPLRLFLIIANTMAFQNDVYEWARDHRAHHKFSETHADPHNSRRGFFFSHVGWLLVRKHPAVKEKGSTLDLSDLEAEKLVMFQRRYYKPGLLLMCFILPTLVPWYFWGETFQNSVFVATFLRYAVVLNATWLVNSAAHLFGYRPYDKNISPRENILVSLGAVGEGFHNYHHSFPYDYSASEYRWHINFTTFFIDCMAALGLAYDRKKVSKAAILARIKRTGDGNYKSG

SEQ ID NO: 98 - protein, 366 aa, Homo sapiens
  ref <308>: DATABASE ACCESSION NUMBER: Genbank / BAD92219
  ref <309>: DATABASE ENTRY DATE: 2006-03-31
  ref <313>: RELEVANT RESIDUES IN SEQ ID NO: (1)..(366)
SRHPRAKMPAHLLQDDISSSYTTTTTITAPPSRVLQNGGDKLETMPLYLEDDIRPDIKDDIYDPTYKDKEGPSPKVEYVWRNIILMSLLHLGALYGITLIPTCKFYTWLWGVFYYFVSALGITAGAHRLWSHRSYKARLPLRLFLIIANTMAFQNDVYEWARDHRAHHKFSETHADPHNSRRGFFFSHVGWLLVRKHPAVKEKGSTLDLSDLEAEKLVMFQRRYYKPGLLLMCFILPTLVPWYFWGETFQNSVFVATFLRYAVVLNATWLVNSAAHLFGYRPYDKNISPRENILVSLGAVGEGFHNYHHSFPYDYSASEYRWHINFTTFFIDCMAALGLAYDRKKVSKAAILARIKRTGDGNYKSG

SEQ ID NO: 99 - protein, 359 aa, Homo sapiens; identical to SEQ ID NO: 97
  ref <308>: DATABASE ACCESSION NUMBER: Genbank / AAH62303
  ref <309>: DATABASE ENTRY DATE: 2005-03-08
  ref <313>: RELEVANT RESIDUES IN SEQ ID NO: (1)..(359)

SEQ ID NO: 100 - protein, 359 aa, Homo sapiens; identical to SEQ ID NO: 97
  ref <308>: DATABASE ACCESSION NUMBER: Genbank / AAD29870
  ref <309>: DATABASE ENTRY DATE: 1999-05-19
  ref <313>: RELEVANT RESIDUES IN SEQ ID NO: (1)..(359)

SEQ ID NO: 101 - protein, 359 aa, Homo sapiens
  ref <308>: DATABASE ACCESSION NUMBER: Genbank / NP_005054
  ref <309>: DATABASE ENTRY DATE: 2006-04-16
  ref <313>: RELEVANT RESIDUES IN SEQ ID NO: (1)..(359)
MPAHLLQDDISSSYTTTTTITAPPSRVLQNGGDKLETMPLYLEDDIRPDIKDDIYDPTYKDKEGPSPKVEYVWRNIILMSLLHLGALYGITLIPTCKFYTWLWGVFYYFVSALGITAGAHRLWSHRSYKARLPLRLFLIIANTMAFQNDVYEWARDHRAHHKFSETHADPHNSRRGFFFSHVGWLLVRKHPAVKEKGSTLDLSDLEAEKLVMFQRRYYKPGLLMMCFILPTLVPWYFWGETFQNSVFVATFLRYAVVLNATWLVNSAAHLFGYRPYDKNISPRENILVSLGAVGEGFHNYHHSFPYDYSASEYRWHINFTTFFIDCMAALGLAYDRKKVSKAAILARIKRTGDGNYKSG

SEQ ID NO: 102 - protein, 355 aa, Homo sapiens
  ref <308>: DATABASE ACCESSION NUMBER: Genbank / AAH05807
  ref <309>: DATABASE ENTRY DATE: 2003-10-03
  ref <313>: RELEVANT RESIDUES IN SEQ ID NO: (1)..(355)
MPAHLLQDDISSSYTTTTTITAPPSRVLQNGGDKLETMPLYLEDDIRPDIKDDIYDPTYKDKEGPSPKVEYVWRNIILMSLLHLGALYGITLIPTCKFYTWLWGVFYYFVSALGITAGAHRLWSHRSYKARLPLRLFLIIANTMAFQNDVYEWARDHRAHHKFSETHADPHNSRRGFFFSHVGWLLVRKHPAVKEKGSTLDLSDLEAEKLVMFQRRYYKPGLLMMCFILPTLVPWYFWGETFQNSVFVATFLRYAVVLNATWLVNSAAHLFGYRPYDKNISPRENILVSLGAVGEGFHNYHHSFPYDYSASEYRWHINFTTFFIDCMAALGLAYDRKKVSKAAILARIKRTGDGN

SEQ ID NO: 103 - protein, 359 aa, Homo sapiens; identical to SEQ ID NO: 101
  ref <308>: DATABASE ACCESSION NUMBER: Genbank / O00767
  ref <309>: DATABASE ENTRY DATE: 2006-04-13
  ref <313>: RELEVANT RESIDUES IN SEQ ID NO: (1)..(359)

SEQ ID NO: 104 - protein, 359 aa, Homo sapiens
  ref <308>: DATABASE ACCESSION NUMBER: Genbank / BAD96582
  ref <309>: DATABASE ENTRY DATE: 2005-04-25
  ref <313>: RELEVANT RESIDUES IN SEQ ID NO: (1)..(359)
MPAHLLQDDISSSYTTTTTITAPPSRVLQNGGDKLETMPLYLEDDIRPDIKDDIYDPTYKDKEGPSPKVEYVWRNIILMSLLHLGALYGITLIPTCKFYTWLWGVFYYFVSALGITAGAHRLWSHRSYKARLPLRLFLIIANTMAFQNDVYEWARDHRAHHKFSETHADPHNSRRGFFFSHVGWLLVRKHPAVKEKGSTLDLSDLEAEKLVMFQRRYYKPGLLMMCFILPTLVPWYFWGETFQNSVFVATFLRYAVVLNATWLVNSAALLFGYRPYDKNISPRENILVSLGAVGEGFHNYHHSFPYDYSASEYRWHINFTTFFIDCMAALGLAYDRKKVSKAAILARIKRTGDGNYKSG

SEQ ID NO: 105 - protein, 237 aa, Homo sapiens
  ref <308>: DATABASE ACCESSION NUMBER: Genbank / AAB30631
  ref <309>: DATABASE ENTRY DATE: 2005-06-24
  ref <313>: RELEVANT RESIDUES IN SEQ ID NO: (1)..(237)
AHMLQEDISSSYTTTTTITAPPPGVLQNGGDKLETMPLYLEDDIRPDIKDDIYDPTYKDKEGPSPKVEYVWRNIILMSLLHLGALYGITLIPTCKFYTWLWGVFYYFVSALGITAGAHRLWSHRSYKARLPLRLFLIIANTMAFQNDVYEWARDHRAHHKFSETHADPHNSRRGFFFSHVGWLLVRKHPAVKEKGSTLDLSDLEAEKLVMFQRRYYKPGLLMMCFILPTLVPWYCWG

SEQ ID NO: 106 - protein, 359 aa, Homo sapiens
  ref <308>: DATABASE ACCESSION NUMBER: Genbank / CAA73998
  ref <309>: DATABASE ENTRY DATE: 2003-08-05
  ref <313>: RELEVANT RESIDUES IN SEQ ID NO: (1)..(359)
MPAHLLQDDISSSYTTTTTITAPPPGVLQNGGDKLETMPLYLEDDIRPDIKDDIYDPTYKDKEGPSPKVEYVWRNIILMSLLHLGALYGITLIPTCKFYTWLWGVFYYFVSALGITAGAHRLWSHRSYKARLPLRLFLIIANTMAFQNDVYEWARDHRAHHKFSETHADPHNSRRGFFFSHVGWLLVRKHPAVKEKGSTLDLSDLEAEKLVMFQRRYYKPGLLMMCFILPTLVPWYFWGETFQNSVFVATFLRYAVVLNATWLVNSAAHLFGYRPYDKNISPRENILVSLGAVGEGFHNYHHSFPYDYSASEYRWHINFNTFFIDWMAALGLTYDRKKVSKAAILARIKRTGDGNYKSG

SEQ ID NO: 107 - protein, 227 aa, Homo sapiens
  ref <308>: DATABASE ACCESSION NUMBER: Genbank / Q13907
  ref <309>: DATABASE ENTRY DATE: 2006-01-24
  ref <313>: RELEVANT RESIDUES IN SEQ ID NO: (1)..(227)
MPEINTNHLDKQQVQLLAEMCILIDENDNKIGAETKKNCHLNENIEKGLLHRAFSVFLFNTENKLLLQQRSDAKITFPGCFTNTCCSHPLSNPAELEESDALGVRRAAQRRLKAELGIPLEEVPPEEINYLTRIHYKAQSDGIWGEHEIDYILLVRKNVTLNPDPNEIKSYCYVSKEELKELLKKAASGEIKITPWFKIIAATFLFKWWDNLNHLNQFVDHEKIYRM

SEQ ID NO: 108 - protein, 284 aa, Homo sapiens
  ref <308>: DATABASE ACCESSION NUMBER: Genbank / NP_004499
  ref <309>: DATABASE ENTRY DATE: 2006-04-16
  ref <313>: RELEVANT RESIDUES IN SEQ ID NO: (1)..(284)
MWRGLALARAIGCAARGRGQWAVRAADCAQSGRHPGPAVVCGRRLISVLEQIRHFVMMPEINTNHLDKQQVQLLAEMCILIDENDNKIGAETKKNCHLNENIEKGLLHRAFSVFLFNTENKLLLQQRSDAKITFPGCFTNTCCSHPLSNPAELEESDALGVRRAAQRRLKAELGIPLEEVPPEEINYLTRIHYKAQSDGIWGEHEIDYILLVRKNVTLNPDPNEIKSYCYVSKEELKELLKKAASGEIKITPWFKIIAATFLFKWWDNLNHLNQFVDHEKIYRM

SEQ ID NO: 109 - protein, 237 aa, Homo sapiens
  ref <308>: DATABASE ACCESSION NUMBER: Gednbank / AAH19227
  ref <309>: DATABASE ENTRY DATE: 2005-08-03
  ref <313>: RELEVANT RESIDUES IN SEQ ID NO: (1)..(237)
VLEQIRHFVMMPEINTNHLDKQQVQLLAEMCILIDENDNKIGAETKKNCHLNENIEKGLLHRAFSVFLFNTENKLLLQQRSDAKITFPGCFTNTCCSHPLSNPAELEESDTLGVRRAAQRRLKAELGIPLEEVPPEEINYLTRIHYKAQSDGIWGEHEIDYILLVRKNVTLNPDPNEIKSYCYVSKEELKELLKKAASGEIKITPWFKIIAATFLFKWWDNLNHLNQFVDHEKIYRM

SEQ ID NO: 110 - protein, 228 aa, Homo sapiens
  ref <308>: DATABASE ACCESSION NUMBER: Genbank / AAK49435
  ref <309>: DATABASE ENTRY DATE: 2001-05-02
  ref <313>: RELEVANT RESIDUES IN SEQ ID NO: (1)..(228)
MMPEINTNHLDKQQVQLLAEMCILIDENDNKIGAETKKNCHLNENIEKGLLHRAFSVFLFNTENKLLLQQRSDAKITFPGCFTNTCCSHPLSNPAELEESDALGVRRAAQRRLKAELGIPLEEVPPEEINYLTRIHYKAQSDGIWGEHEIDYILLVRKNVTLNPDPNEIKSYCYVSKEELKELLKKAASGEIKITPWFKIIAATFLFKWWDNLNHLNQFVDHEKIYRM

SEQ ID NO: 111 - protein, 228 aa, Homo sapiens; identical to SEQ ID NO: 110
  ref <308>: DATABASE ACCESSION NUMBER: Genbank 7 AAK49434
  ref <309>: DATABASE ENTRY DATE: 2001-05-02
  ref <313>: RELEVANT RESIDUES IN SEQ ID NO: (1)..(228)

SEQ ID NO: 112 - protein, 228 aa, Homo sapiens; identical to SEQ ID NO: 110
  ref <308>: DATABASE ACCESSION NUMBER: Genbank / AAK29357
  ref <309>: DATABASE ENTRY DATE: 2001-04-04
  ref <313>: RELEVANT RESIDUES IN SEQ ID NO: (1)..(228)

SEQ ID NO: 113 - protein, 237 aa, Homo sapiens
  ref <308>: DATABASE ACCESSION NUMBER: Genbank / AAH06999
  ref <309>: DATABASE ENTRY DATE: 2005-08-03
  ref <313>: RELEVANT RESIDUES IN SEQ ID NO: (1)..(237)
VLEQIRHFVMMPEINTNHLDKQQVQLLAEMCILIDENDNKIGAETKKNCHLNENIEKGLLHRAFSVFLFNTENKLLLQQRSDAKITFPGCFTNTCCSHPLSNPAELEESDALGVRRAAQRRLKAELGIPLEEVPPEEINYLTRIHYKAQSDGIWGEHEIDYILLVRKNVTLNPDPNEIKSYCHVSKEELKELLKKAASGEIKITPWFKIIAATFLFKWWDNLNHLNQFVDHEKIYRM

SEQ ID NO: 114 - DNA, 27 bp, Artificial
  Primer
aagccatcttccttagcctcccaagta

SEQ ID NO: 115 - DNA, 27 bp, Artificial
  Primer
aaaaccctgtctccacacacacaaaaa

SEQ ID NO: 116 - DNA, 23 bp, Artificial
  Primer
ctttgggttcatttgctgttttg

SEQ ID NO: 117 - DNA, 25 bp, Artificial
  Primer
cattagaaaaagagagctgggtgta

SEQ ID NO: 118 - DNA, 25 bp, Artificial
  Primer
gctattcacagatgcgaacatagta

SEQ ID NO: 119 - DNA, 25 bp, Artificial
  Primer
ggagagtgatagacaaagttctgga

SEQ ID NO: 120 - DNA, 27 bp, Artificial
  Primer
tggctgttatttaggactctgtggaaa

SEQ ID NO: 121 - DNA, 27 bp, Artificial
  Primer
tcccccactccttcacttaaggtataa

SEQ ID NO: 122 - DNA, 27 bp, Artificial
  Primer
cttattcagggacaagctgaaacacat

SEQ ID NO: 123 - DNA, 28 bp, Artificial
  Primer
agtgtttcctctttgacttcctcatttt

SEQ ID NO: 124 - DNA, 25 bp, Artificial
  Primer
aattaactgccctatccaaaaagac

SEQ ID NO: 125 - DNA, 25 bp, Artificial
  Primer
cagaagagactttggtttttgtcat

SEQ ID NO: 126 - DNA, 25 bp, Artificial
  Primer
aaagatgatatatatgaccccacct

SEQ ID NO: 127 - DNA, 25 bp, Artificial
  Primer
ccaagtgtagcagagacataaggat